(19) United States
(12) Patent Application Publication
(10) Pub. No.: US 2008/0317795 A1
(43) Pub. Date: Dec. 25, 2008

(54) HIGHLY CHARGED MICROCAPSULES

(76) Inventors: Daniel Henry TRAYNOR, Sarasota, FL (US); Henry G. Traynor, Sarasota, FL (US); Steven M. Markowitz, Honolulu, HI (US); David L. Compton, Ventura, CA (US)

Correspondence Address: WILSON SONSINI GOODRICH & ROSATI, 650 PAGE MILL ROAD, PALO ALTO, CA, 94304-1050, US

(21) Appl. No.: 12/125,035

(22) Filed: May 21, 2008

Related U.S. Application Data
(60) Provisional application No. 60/939,318, filed on May 21, 2007.

(51) Int. Cl. A61K 8/00; A61Q 17/04

(52) U.S. Cl. 424/401; 424/59

(57) ABSTRACT

The invention encompasses compositions containing sol-gel microcapsules that are highly positively charged. The sol-gel capsules generally contain additives. The invention also encompasses methods for producing highly charged microcapsules using cationic additives which can include cationic polymers. The methods allow for the encapsulation of polar or non-polar active ingredients.

### CROSS-REFERENCE

This application claims the benefit U.S. Provisional Application No. 60/939,318 filed May 21, 2007, which is incorporated herein by reference in its entirety.

### BACKGROUND OF THE INVENTION

Exposure to ultraviolet light, primarily through exposure to the sun's rays, produces a number of harmful effects including premature skin aging, loss of elasticity, wrinkling, drying, and an increased risk of developing skin cancer. Currently a number of sunscreen products are marketed to protect against these harmful effects. All of these products contain agents known to filter out some of the sun's harmful rays incorporated into creams, ointments, lotions, solutions or suspensions. Such products are generally applied just prior to anticipated sun exposure, provide short term protection, and are removed by bathing, washing or normal desquamation of skin. Soap in the form of bodywash has for years been used to remove oil due to its surfactant composition and associated charges. A normal soap contains both charges of a positive and negative nature. Although attempts have been made to combine sunscreens with soaps (i.e., surfactant agents), none has provided an ideal combination of high sun protection factor (SPF) and long-lasting effect in a composition that maintains its integrity.

Other additives in addition to sunscreen are also potentially useful when applied in to surfaces either as a wash-on or as a leave-on formulation. Other additives in addition to sunscreens are also useful when applied to a surface as a crime, gel, lotion, shampoo, conditioner, coating, spray, or as a bath bar. One approach to providing active ingredients to surfaces including topical preparations is to encapsulate the additive in order to protect the additive, control the release of the additive, modify the function of the additive, and in some cases to prevent the additive from harming the surface, which in some cases is skin. In addition to functional additives useful for the skin, the application of functional additives to the surfaces of plants and on other substrates such as textiles, walls, floors, cars, trucks, and boats is also important. Methods of encapsulation, such as sol-gel encapsulation are known in the art, but there is a need for improved encapsulated additives that have stability and the ability to effectively bind and to release at the appropriate time when applied either to the body, or to other substrates. The present invention addresses these needs.

### SUMMARY OF THE INVENTION

One aspect of the invention provides for highly charged sol-gel capsules that are useful for applications on a variety of surfaces. In one aspect, the invention provides a sol-gel microcapsule with a zeta potential of at least about 40 mV. In some embodiments the zeta potential is at least about 50 mV. In some embodiments the zeta potential is at least about 55 mV. In some embodiments the zeta potential is at least about 60 mV.

One aspect of the invention is a plurality of sol-gel microcapsules capable of binding to a surface wherein an average of at least about 50% of the microcapsules remain bound to the surface for an average of greater than at least about 4 hours.

One aspect of the invention is a sol-gel microcapsule with a zeta potential of at least about 40 mV wherein the microcapsule comprises a cationic agent. In some embodiments the cationic agent comprises a cationic polymer. In some embodiments the cationic polymer comprises polyquaternium-4, -7, -11, -22, -27, -44, 51, or -64. In some embodiments the cationic polymer comprises polyquaternium-4.

In some embodiments sol-gel microcapsule of claims 1-4, wherein the microcapsule is associated with an additive. In some embodiments is encapsulated in the microcapsule. In some embodiments the additive is located substantially within the sol-gel microcapsule.

In some embodiments the additive is selected from the group consisting of steroidal anti-inflammatory actives, analgesic actives, antifungals, antibacterials, antiparasitics, anti-virals, anti-allergenics, anti-cellulite additives, medicinal actives, skin rash, skin disease and dermatitis medications, insect repellant actives, antioxidants, hair growth promoter, hair growth inhibitor, hair bleaching agents, deodorant compounds, sunless tanning actives, skin lightening actives, anti-acne actives, anti-skin wrinkling actives, anti-skin aging actives, vitamins, nonsteroidal anti-inflammatory actives, anesthetic actives, anti-pruritic actives, anti-microbial actives, dental care agents, personal care agents, nutraceuticals, pharmaceuticals, fragrances, antifouling agents, pesticides, lubricants, etchants, and mixtures and combinations thereof.

The highly charged microcapsules can be used for agricultural, textile, industrial, transportation, marine, pharmaceutical, or personal care applications.

In some embodiments the additive is selected from the group consisting of sunscreens, skin lightening actives, anti-aging additives, fragrances, pharmaceuticals, antibacterials, moisturizers, anti-acne actives, and insect repellants. In some embodiments the additive comprises a sunscreen. In some embodiments the sunscreen is selected from the group consisting of aminobenzoic acid, avobenzone, cinnoxate, dioxybenzone, homosalate, menthyl anthranilate, octocrylene, octyl methoxycinnamate, octyl salicylate, oxybenzone, padimate O, phenylbenzimidazole sulfonic acid, sulisobenzone, and trolamine salicylate. In some embodiments the sunscreen comprises a UVA-absorbing sunscreen, a UVB-absorbing sunscreen, and a physical blocker sunscreen. In some embodiments (i) the UVB-absorber sunscreen is selected from the group consisting of aminobenzoic acid, cinoxate, dioxybenzone, homosalate, octocrylene, octyl methoxycinnamate, octyl salicylate, oxybenzone, padimate O, phenylbenzimidazole sulfonic acid, sulisobenzone, and trolamine salicylate; (ii) the UVA-absorber sunscreen is selected from the group consisting of avobenzone and menthyl anthranilate; and (iii) the physical blocker sunscreen is selected from the group consisting of titanium dioxide and zinc oxide.

One aspect of the invention is composition comprising a highly charged microcapsule and further comprising a vehicle suitable for treatment of surfaces in topical, agricultural, textile, industrial, transportation, marine, pharmaceutical, or personal care uses. In some embodiments the composition comprises a wash-on product. In some embodiments the composition comprises a leave-on product. In some embodiments the microcapsules in the composition experience an average of greater than about 50% breakage when applied to the surface. In some embodiments the breakage substantially occurs on initial application to the surface. In some embodiments the average of greater than 50% breakage occurs over a period of about 1 hour. In some embodiments the average of greater than 50% breakage occurs over a period of about 6 hours. In some embodiments the average of greater than 50% breakage occurs over a period of about 12 hours. In some embodiments the average of greater than 50% breakage occurs over a period of about 24 hours.

In some embodiments the breakage occurs due to the conditions of surface application. In some embodiments the condition of surface application is friction, pressure, light, pH change, or enzymatic action.

One aspect of the invention is a method of applying an active compound to a surface comprising; providing a composition comprising an active compound encapsulated into a sol-gel microcapsule having a zeta potential of greater than about 30 mV; and applying the composition to the surface. In some embodiments the zeta potential is greater than 30 mV. In some embodiments the zeta potential is greater than 40 mV. In some embodiments the zeta potential is greater than 55 mV. In some embodiments the zeta potential is greater than 60 mV.

In some embodiments the capsules comprise a cationic polymer. In some embodiments the cationic polymer comprises a polyquaternium. In some embodiments the cationic polymer comprises polyquatemium-4, -7, -11, -22, -27, -44, 51, or -64.

In some embodiments the additive is selected from the group consisting of steroidal anti-inflammatory actives, analgesic actives, antifungals, antibacterials, antiparasitics, anti-virals, anti-allergenics, anti-cellulite additives, medicinal actives, skin rash, skin disease and dermatitis medications, insect repellant actives, antioxidants, hair growth promoter, hair growth inhibitor, hair bleaching agents, deodorant compounds, sunless tanning actives, skin lightening actives, anti-acne actives, anti-skin wrinkling actives, anti-skin aging actives, vitamins, nonsteroidal anti-inflammatory actives, anesthetic actives, anti-pruritic actives, anti-microbial actives, dental care agents, personal care agents, nutraceuticals, pharmaceuticals, fragrances, antifouling agents, pesticides, lubricants, etchants, and mixtures and combinations thereof.

In some embodiments the additive is selected from the group consisting of sunscreens, skin lightening actives, anti-aging additives, fragrances, pharmaceuticals, antibacterials, moisturizers, anti-acne actives, and insect repellants. In some embodiments the additive comprises a sunscreen. In some embodiments the sunscreen is selected from the group consisting of aminobenzoic acid, avobenzone, cinnoxate, dioxybenzone, homosalate, menthyl anthranilate, octocrylene, octyl methoxycinnamate, octyl salicylate, oxybenzone, padimate O, phenylbenzimidazole sulfonic acid, sulisobenzone, and trolamine salicylate. In some embodiments the sunscreen comprises a UVA-absorbing sunscreen, a UVB-absorbing sunscreen, and a physical blocker sunscreen. In some embodiments (i) the UVB-absorber sunscreen is selected from the group consisting of aminobenzoic acid, cinoxate, dioxybenzone, homosalate, octocrylene, octyl methoxycinnamate, octyl salicylate, oxybenzone, padimate O, phenylbenzimidazole sulfonic acid, sulisobenzone, and trolamine salicylate; (ii) the UVA-absorber sunscreen is selected from the group consisting of avobenzone and menthyl anthranilate; and (iii) the physical blocker sunscreen is selected from the group consisting of titanium dioxide and zinc oxide.

In some embodiments the microcapsules in the composition experience an average of greater than about 50% breakage when applied to the surface. In some embodiments the breakage substantially occurs on initial application to the surface. In some embodiments the breakage occurs over a period of 1 hour. In some embodiments the breakage occurs over a period of 6 hours. In some embodiments the breakage occurs over a period of 12 hours. In some embodiments the breakage occurs over a period of 24 hours.

One aspect of the invention is a method of manufacturing a highly charged sol-gel microcapsule comprising a non-polar active ingredient comprising: (a) combining the non-polar active ingredient, optional non-polar diluent, and aqueous phase; (b) agitating the combination formed in (a) to form an oil-in-water (O/W) emulsion wherein the non-polar active ingredient and optional non-polar diluent comprise the dispersed phase; (c) adding one or more surfactants; (d) adding a cationic agent; (e) adding a gel precursor to the O/W emulsion; and (f) mixing the composition from step (e) while the gel precursor hydrolyzes and sol-gel capsules are formed which comprise the non-polar active ingredient.

In some embodiments the method further comprises step (g) filtering the sol-gel microcapsules and step (h) rinsing the sol-gel microcapsules.

In some embodiments the method further comprises step (i) drying the microcapsules.

In some embodiments the method of manufacturing produces a microcapsule having zeta potential of at least about 30 mV. In some embodiments the method of manufacturing produces a microcapsule having a zeta potential of at least about 40 mV. In some embodiments the method of manufacturing produces a microcapsule zeta potential of at least about 55 mV. In some embodiments the method of manufacturing produces a microcapsule having zeta potential of at least about 60 mV.

In some embodiments the steps are carried out in the order listed. In some embodiments the cationic agent is added after the addition of the gel precursor. In some embodiments the cationic agent is added during step (f). In some embodiments the cationic agent is added after step (f). In some embodiments cationic agent is added during step (h) of rinsing the sol-gel microcapsules. In some embodiments the cationic agent is added after step (i) of drying the sol-gel microcapsules. In some embodiments the cationic agent comprises a cationic polymer. In some embodiments the cationic polymer comprises polyquaternium-4, -7, -11, -22, -27, -44, 51, or -64. In some embodiments the cationic polymer comprises polyquaternium-4. In some embodiments the cationic agent comprises a proton donor.

In some embodiments step (f) is carried out at acidic pH. In some embodiments step (f) is carried out at a pH from 3.6 to 4.0. In some embodiments the one or more surfactants comprises a copolymer surfactant. In some embodiments the one or more surfactants have a combined hydrophile-lipophile balance (HLB) of between 9 and 11.

One aspect of the invention is a method of manufacturing a highly charged sol gel microcapsule comprising a polar active ingredient comprising: (a) combining the polar active ingredient, water, optional polar diluent, and a non-polar (oil) phase; (b) agitating the combination formed in (a) to form an water-in-oil (W/O) emulsion wherein the polar active ingredient, water, and optional polar diluent comprise the dispersed phase; (c) adding one or more surfactants; (d) adding a cationic agent; (e) adding a gel precursor to the W/O emulsion; and (f) mixing the composition from step (e) while the gel precursor hydrolyzes and sol-gel capsules are formed which comprise the polar active ingredient.

In some embodiments the method further comprises step (g) filtering the sol-gel microcapsules and step (h) rinsing the sol-gel microcapsules.

In some embodiments the method further comprises step (i) drying the microcapsules.

In some embodiments the method of manufacturing produces a microcapsule having zeta potential of at least 30 mV. In some embodiments the method of manufacturing produces a microcapsule having a zeta potential of at least 40 mV. In some embodiments method of manufacturing produces a microcapsule zeta potential of at least 55 mV. In some embodiments the method of manufacturing produces a microcapsule having zeta potential of at least 60 mV.

In some embodiments the steps are carried out in the order listed. In some embodiments the cationic agent is added after the addition of the gel precursor In some embodiments the cationic agent is added during step (f). In some embodiments the cationic agent is added after step (f). In some embodiments the cationic agent is added during step (h) of rinsing the sol-gel microcapsules. In some embodiments the cationic agent is added after step (i) of drying the sol-gel microcapsules.

In some embodiments the cationic agent comprises a cationic polymer. In some embodiments the cationic polymer comprises polyquaternium-4, -7, -11, -22, -27, -44, 51, or -64. In some embodiments the cationic polymer comprises polyquaternium-4. In some embodiments the cationic agent comprises a proton donor.

In some embodiments step (f) is carried out at acidic pH. In some embodiments step (f) is carried out at a pH from 3.6 to 4.0. In some embodiments the one or more surfactants comprises a copolymer surfactant. In some embodiments the one or more surfactants have a combined hydrophile-lipophile balance (HLB) of between 2 and 6.

One aspect of the invention is a method of forming a highly charged sol-gel microcapsule comprising an active ingredient within a template comprising: (a) forming a dispersion of templates, wherein the templates comprise an active ingredient, in an aqueous continuous phase; (b) adding a cationic agent; (c) adding a gel precursor to the aqueous continuous phase; and (d) mixing the composition from step (c) while the gel precursor hydrolyzes and sol-gel capsules are formed.

In some embodiments the method further comprises step (e) filtering the sol-gel microcapsules and step (f) rinsing the sol-gel microcapsules.

In some embodiments the method further comprises step (g) drying the microcapsules.

In some embodiments the method of manufacturing produces a microcapsule having zeta potential of at least 30 mV. In some embodiments the method of manufacturing produces a microcapsule having a zeta potential of at least 40 mV. In some embodiments the method of manufacturing produces a microcapsule zeta potential of at least 55 mV. In some embodiments the method of manufacturing produces a microcapsule having zeta potential of at least 60 mV.

In some embodiments the steps are carried out in the order listed. In some embodiments the cationic agent is added after the addition of the gel precursor. In some embodiments the cationic agent is added during step (c). In some embodiments the cationic agent is added after step (c). In some embodiments the cationic agent is added during step (f) of rinsing the sol-gel microcapsules. In some embodiments the cationic agent is added after step (g) of drying the sol-gel microcapsules.

In some embodiments the cationic agent comprises a cationic polymer. In some embodiments the cationic polymer comprises polyquaternium-4, -7, -11, -22, -27, -44, 51, or -64. In some embodiments the cationic polymer comprises polyquaternium-4. In some embodiments the cationic agent comprises a proton donor.

In some embodiments step (d) is carried out at acidic pH. In some embodiments step (d) is carried out at a pH from 3.6 to 4.0. In some embodiments the template comprises a microsphere. In some embodiments the template comprises a polymer, liposome or micelle. In some embodiments the template comprises a phospholipid.

### INCORPORATION BY REFERENCE

All publications and patent applications mentioned in this specification are herein incorporated by reference to the same extent as if each individual publication or patent application was specifically and individually indicated to be incorporated by reference.

### DETAILED DESCRIPTION OF THE INVENTION

The present invention encompasses compositions containing one or more additives that can be active ingredients (also referred to herein as “actives”) that may be added to the highly charged sol-gel microcapsule containing composition, for example, to provide an active in either a leave-on or a wash-on formulation. A wash on formulation can include an active/bodywash combination. The invention also encompasses a bodywash containing such an active ingredient. In some embodiments, the active ingredient is one or more sunscreens. In some embodiments, the highly charged microcapsules are used for agricultural, textile, industrial, transportation, marine, pharmaceutical, or personal care applications. The highly charged microcapsules generally comprise an active agent within the microcapsule. In some cases, the active agent can perform its function while contained within the microcapsule. In some cases, the active agent must leave the microcapsule in order to perform its action. In some embodiments, the capsules are produced such that the capsules rupture in order to release the active ingredient. The cationic component can act to facilitate the controlled breakage of the capsules. In some cases, the surface onto which the capsules are applied is pre-coated with an agent that reacts with the sol-gel capsule in order to cause controlled breakage of the capsules and release of the active ingredient. In some cases the surface can be post treated with a substance that either enhances or retards capsule breakage. The invention further encompasses methods of use and manufacture of the compositions, and business methods.

A used herein, a “wash-on” formulation encompasses all cleansing vehicles applied to a surface. A wash-on formulation is generally applied to a surface in order to perform a cleaning function, and in addition to the cleaning aspect of the wash-on, a portion of the wash-on formulation remains on the surface to provide a function beyond cleaning. Exemplary forms of cleansing vehicles include, but are not limited to, liquid, bar, gel, foam, aerosol or pump spray, cream, lotion, stick, powder, or incorporated into a patch or a towelette. In addition, soapless cleansers may be used as well. The wash-on can be made into any suitable product form.

As used herein, a “leave-on” formulation is applied directly to a surface. A leave-on formulation may not perform a cleansing function. The leave-on can be, for example, a cream, lotion, gel, coating, paint, varnish, oil, spray, or powder. The leave-on formulations of the invention generally have a function that is performed or enhanced by the active that is delivered to the surface within the highly charged sol-gel capsules.

As used herein, “bodywash” is a type of wash-on formulation that encompasses all cleansing vehicles applied to the body. Exemplary forms of cleansing vehicles include, but are not limited to, liquid, bar, gel, foam, aerosol or pump spray, cream, lotion, stick, powder, or incorporated into a patch or a towelette. In addition, soapless cleansers may be used as well. The bodywash can be made into any suitable product form. Thus, as used herein, “bodywash” includes, but is not limited to, a soap including liquid and bar soap; a shampoo; a hair conditioner; a shower gel; including an exfoliating shower gel; a foaming bath product (e.g. gel, soap or lotion); a milk bath; a soapless cleanser, including a gel cleanser, a liquid cleanser and a cleansing bar; moist towelletes; a body lotion; a body spray, mist or gel; bath effervescent tablets (e.g., bubble bath); a hand and nail cream; a bath/shower gel; a shower cream; a depilatory cream; a shaving product e.g. a shaving cream, gel, foam or soap, an after-shave, after-shave moisturizer; and combinations thereof, and any other composition used for cleansing or post-cleansing application to the body, including the skin and hair. Especially useful as bodywashes in the invention are soaps, e.g., liquid soaps and bar soaps, and shampoos.

### I. Compositions

The highly charged microcapsules of the invention are used to produce compositions for agricultural, textile, industrial, transportation, marine, pharmaceutical, or personal care applications. The compositions can be applied to a broad range of surfaces. The highly charged microcapsules contain additives or active ingredients that perform a function when applied as part of the compositions of the present invention.

In one aspect, the invention provides additives containing active ingredients, where the additive is designed to be added to a leave-on or wash-on product such as a bodywash (e.g., soap or shampoo). In some embodiments, the invention provides sunscreen compositions (“sunscreen additives”) that may be added to a bodywash preparation to impart sun protection. In some embodiments, the invention provides a combination of a sunscreen additive and a bodywash preparation (“sunscreen/bodywash”). Thus, a sunscreen additive of the invention may be mixed with a conventional bodywash; alternatively, the invention provides pre-mixed sunscreen/bodywash. In either case, the sunscreen/bodywash composition is generally applied in the same manner as the bodywash alone and, typically, rinsed, with additive, e.g., sunscreen protection, being left on the skin after rinsing. In some cases, e.g., soapless cleansers, the bodywash is applied without rinsing. For sunscreen additives as part of a sunscreen/bodywash, the sunscreen protection after application and, typically, rinsing is, on average, greater than an SPF of 1, up to about SPF 50. As used herein in the context of SPF, “average SPF” is the SPF, determined as described herein, for about 5 to about 50 subjects, or about 5 to about 20 subjects, or about 5 to about 10 subjects, where the subjects preferably have Type II skin. In some embodiments, the average SPF provided by the sunscreen/bodywash after rinsing is about 1 to about 50, or about 2 to about 50, or about 2 to about 40, or about 2 to about 30, or about 2 to about 20, or about 2 to about 10, or about 2 to about 5, or about 5 to about 25, or about 5 to about 20, or about 5 to about 15, or about 5 to about 10. In some embodiments, the average SPF provided by the sunscreen/bodywash, after rinsing, is above about 1, 2, 3, 4, 5, 6, 7, 8, 9, 10, 11, 12, 13, 14, 15, 16, 17, 18, 19, or 20. In some embodiments, the average SPF after rinsing is above about 2. In some embodiments, the average SPF after rinsing is above about 5. In some embodiments, the average SPF after rinsing is above about 10. In some embodiments, the average SPF after rinsing is above about 15. In some embodiments, the average SPF provided by the sunscreen/bodywash of the invention remains above about 1, 2, 3, 4, 5, 6, 7, 8, 9, 10, 11, 12, 13, 14, 15, 16, 17, 18, 19, or 20, for an average of at least about 0.5, 1, 2, 3, 4, 5, 6, 7, 8, 9, 10, or more than about 10 hours after rinsing. In some embodiments the average SPF provided by the sunscreen/bodywash of the invention increases with each additional washing after a first wash, so that after a second, third, fourth, or fifth wash, the SPF provided can be above about 2, 4, 6, 8, 10, 15, 20, 25, 30, 40, 45, or more than about 45.

SPF is a commonly used measure of photo protection of a sunscreen against erythema. This number is derived from another parameter, the minimal erythemal dose (MED). MED is defined as the “least exposure dose at a specified wavelength that will elicit a delayed erythema response.” The MED indicates the amount of energy irradiating the skin and the responsiveness of the skin to the radiation. The SPF of a particular photo protector is obtained by dividing the MED of protected skin by the MED of unprotected skin. The higher the SPF, the more effective the agent in preventing sunburn. The SPF value tells how many times longer a person can stay in the sun before the person will experience 1 MED. For example, utilizing a sunscreen with an SPF of 6 will allow an individual to stay in the sun six times longer before receiving 1 MED. As the SPF value of a sunscreen increases, the less chance exists for development of tanning of the skin. Typically, commercially available sunscreening products have SPF values ranging from about 2 to 45.

Methods for measuring SPF are described in, e.g., FDA monograph 21 C.F.R. 352. In order to determine SPF, the procedures of the FDA monograph can be used. Another useful method for applying the sunscreen prior to measurement is as follows: Wet 50 cm² square area of testing site with 10 ml of water delivered with a syringe. Apply test sample as per FDA monograph to area. Work lather on the subject for 3 minutes to allow the product to absorb into the skin. Rinse area after 2 additional minutes with 20 ml of water. Pat dry and allow 15 minutes before exposure to radiation as per FDA monograph.

The sol-gel capsules of the invention can be formulated to control whether or not there is penetration into the skin or other surface and if there is penetration, to what depth. In some cases the control of penetration can be influenced by the conditions of the skin such as pH, presence of film formers, and roughness. Where sunscreens are used, penetration into the skin is not generally desirable and the capsules can be formulated to minimize or eliminate skin penetration. In some embodiments, such as where the active ingredient is a pigment or pharmaceutical on the skin, some amount of skin penetration is desired. In some embodiments, after application of the bodywash containing the additive to the skin followed by rinsing, the additive penetrates to an average of at least about 5 microns beneath the skin surface. The capsules can be formulated such that the active will penetrate only to a given layer of the skin. The skin can be seen to have three primary layers, the epidermis, which provides waterproofing and serves as a barrier to infection; the dermis, which serves as a location for the appendages of skin; and the hypodermis (subcutaneous adipose layer). In some embodiments the active ingredient penetrates the epidermis. In some embodiments the active ingredient penetrates the dermis. In some embodiments the active ingredient penetrates the hypodermis. The capsules can thus be produced such that the contents of the capsules, the active ingredients, are introduced into the blood stream. In some embodiments, the additive penetrates to an average of at least about 10, 15, 20, 25, 30, 40, 50, 60, 70, 80, 90, 100, 120, or 150 microns beneath the skin surface. In some embodiments, after application of the leave-on or bodywash containing the additive to the skin followed by rinsing, the additive penetrates to an average of no more than about 30 microns beneath the skin surface. In some embodiments, the additive penetrates to an average of no more than about 50, 40, 30, 25, 20, 15, 10, or 5 microns beneath the skin surface. In some embodiments, after application of the bodywash containing the additive to the skin followed by rinsing, the additive penetrates to an average of about 5 to about 50, or about 5 to about 40, or about 5 to about 30, or about 10 to about 40, or about 15 to about 40, or about 20 to about 40, or about 5, 10, 15, 20, 25, 30, 25, 40, 45, or 50 microns beneath the skin surface. Depth of penetration may be tested by tape stripping methods, as are well-known in the art. In some embodiments, the highly charged material in the capsules can assist in disrupting cell membranes in order to actively deliver active agents into the tissue or the blood. In some embodiments the highly charged material will be inert to the skin and will not cause disruption and penetration.

The sunscreen additives and sunscreen/bodywashes of the invention contain at least one sunscreen. In some embodiments, the sunscreen additives of the invention contain one, two, three, four, or more than four sunscreens. In some embodiments, the sunscreen additives of the invention include three sunscreens. In other embodiments, the sunscreen additives of the invention include four sunscreens. The sunscreens may be organic or inorganic. The sunscreens may be a UVA absorber, a UVB absorber, a physical blocker, or any combination thereof. In some embodiments one or more of the sunscreens is encapsulated. A number of types of encapsulation may be employed as described herein.

Compositions of the invention may include one or more actives that are not sunscreens, where the composition is designed to be an additive to a bodywash. In some compositions of the invention, the actives are provided in combination with one or more sunscreens. In some compositions, the actives are provided without sunscreen.

The compositions, e.g., sunscreen additives, and additive/bodywashes, e.g., sunscreen/bodywashes, of the invention may further include one or more components to provide a positive charge to the system to assist with attachment to protein and other charged components of skin and/or hair, e.g., cationic polymeric agents. The cationic polymer may be, for example, a quaternium, e.g., polyquaternium.

The additives, e.g., sunscreen additives, and additive/bodywashes, e.g., sunscreen/bodywashes, of the invention may further include a film former.

Other optional ingredients of the additives, e.g., sunscreen additives, and additive/bodywashes, e.g., sunscreen/bodywashes, of the invention include preservatives, antioxidants, chelating agents, liquid hydrocarbon (e.g., similar to pentane), foaming agents (e.g., a cationic foaming agent), skin nourishing components, antibacterials, medicinals, and the like, as described below.

The additives, e.g., sunscreen additives, of the invention may be combined with any conventional bodywash. The bodywash composition with which the additive, e.g., sunscreen additive is combined may be any bodywash known in the art or apparent to one of skill in the art, as described above. In embodiments where the additive is a non-sunscreen active, the additive may be combined with any composition intended for topical application. In these embodiments, the additive is often encapsulated, e.g., in sol-gel microcapsules.

In some embodiments the invention provides an additive, e.g., sunscreen additive, in combination with a bodywash composition to provide an additive/bodywash, e.g. sunscreen/bodywash, composition. In these embodiments, the additive, e.g., sunscreen additives, of the invention are provided in combination with one or more surfactants. The surfactant(s) may be cationic, anionic, nonionic, zwitterionic, amphoteric, or any combination thereof. In some embodiments, the sunscreen/bodywash compositions of the invention include at least one cationic surfactant.

A. Sunscreens

The sunscreen additives and sunscreen/bodywashes of the invention contain at least one sunscreen. The sunscreen may be organic or inorganic, or a combination of both may be used. Sunscreens of use in the invention include UV absorbers or blockers (e.g., many inorganic sunscreens are UV blockers). UV absorbers may be a UVB or UVA absorber (e.g., UVA I or UVA II absorber). In some embodiments, the sunscreen additives or sunscreen/bodywashes of the invention include an organic and an inorganic sunscreen. In some embodiments, the sunscreen additives or sunscreen/bodywashes of the invention include more than one organic sunscreen (e.g., at least one UVB absorber and at least one UVA absorber) and at least one inorganic sunscreen. In some embodiments, the sunscreen additives of the invention include only a physical blocker sunscreen, e.g., titanium dioxide. These embodiments may further contain a cationic polymer and/or a film former, as well as any other components described herein for sunscreen additives.

Additional ingredients may include film formers, cationic polymers, antioxidants, preservatives, and the like, as described herein. In some embodiments, the sunscreen additives or sunscreen/bodywashes of the invention include an organic and an inorganic sunscreen. In some embodiments, the sunscreen additives or sunscreen/bodywashes of the invention include more than one organic sunscreen (e.g., at least one UVB absorber and at least one UVA absorber) and at least one inorganic sunscreen.

In some embodiments, one or more of the sunscreens used in the invention are encapsulated.

Any sunscreen known in the art or apparent to the skilled artisan may be used in the invention. The term “sunscreen” or “sunscreen agent” as used herein defines ultraviolet ray-blocking compounds exhibiting absorption or blockage within the wavelength region between about 290 and 420 nm. Sunscreens may be classified into five groups based upon their chemical structure: para-amino benzoates; salicylates; cinnamates; benzophenones; and miscellaneous chemicals including menthyl anthralinate and digalloyl trioleate. Inorganic sunscreens may also be used including titanium dioxide, zinc oxide, iron oxide and polymer particles such as those of polyethylene and polyamides.

Specific suitable sunscreens include, for example: p-aminobenzoic acid, its salts and its derivatives (ethyl, isobutyl, glyceryl esters; p-dimethylaminobenzoic acid); Anthranilates (i.e., o-aminobenzoates; methyl, menthyl, phenyl, benzyl, phenylethyl, linalyl, terpinyl, and cyclohexenyl esters); Salicylates (amyl, phenyl, benzyl, menthyl, glyceryl, and dipropylene glycol esters); Cinnamic acid derivatives (methyl and benzyl esters, alpha-phenyl cinnamonitrile; butyl cinnamoyl pyruvate); Dihydroxycinnamic acid derivatives (umbelliferone, methylumbelliferone, methylaceto-umbelliferone); Trihydroxycinnamic acid derivatives (esculetin, methylesculetin, daphnetin, and the glucosides, esculin and daphnin); Hydrocarbons (diphenylbutadiene, stilbene); Dibenzalacetone and benzalacetophenone; Naphtholsulfonates (sodium salts of 2-naphthol-3,3-disulfonic and of 2-naphthol-6,8-disulfonic acids); Dihydroxynaphthoic acid and its salts; o- and p-Hydroxybiphenyldisulfonates; Coumarin derivatives (7-hydroxy, 7-methyl, 3-phenylyll); Diazoles (2-acetyl-3-bromoindazole, phenyl benzoxazole, methyl naphthoxalole, various aryl benzothiazoles); Quinine salts (bisulfate, sulfate, chloride, oleate, and tannate); quinoline derivatives (8-hydroxyquinoline salts, 2-phenylquinoline); Hydroxy- or methoxy substituted benzophenones; Uric and vilouric acids; Tannnic acid and its derivatives (e.g., hexaethylether); (Butyl carbityl) (6-propyl piperonyl)ether; Hydroquinone; Benzophenones (Oxybenzene, Sulisobenzone, Dioxybenzone, Benzoresorcinol, 2,2′,4,4′-Tetrahydroxybenzophenone, 2,2′-Dihydroxy-4,4′-dimethoxybenzophenone, Octabenzone; 4-Isopropyhldibenzoylmethane; Butylmethoxydibenzoylmethane; Etocrylene; and 4-isopropyl-di-benzoylmethane; titanium dioxide, iron oxide, zinc oxide, and mixtures thereof. Other cosmetically-acceptable sunscreens and concentrations (percent by weight of the total cosmetic sunscreen composition) include diethanolamine methoxycinnamate (10% or less), ethyl-[bis(hydroxypropyl)]aminobenzoate (5% or less), glyceryl aminobenzoate (3% or less), 4-isopropyl dibenzoylmethane (5% or less), 4-methylbenzylidene camphor (6% or less), terephthalylidene dicamphor sulfonic acid (10% or less), and sulisobenzone (also called benzophenone-4, 10% or less).

In some embodiments, sunscreens are FDA-approved or approved for use in the European Union. For example, FDA-approved sunscreens may be used, singly, or in combination. See, e.g., U.S. Pat. Nos. 5,169,624; 5,543,136; 5,849,273; 5,904,917; 6,224,852; 6,217,852; and Segarin et al., chapter Vil, pages 189 of Cosmetics Science and Technology, and Final Over-the-Counter Drug Products Monograph on Sunscreens (Federal Register, 1999: 64:27666-27963), all of which are incorporated herein by reference.

For example, for a product marketed in the United States, preferred cosmetically-acceptable sunscreens and concentrations (reported as a percentage by weight of the total cosmetic sunscreen composition, and referring to the final percentage of the sunscreen after addition to the bodywash) include: aminobenzoic acid (also called para-aminobenzoic acid and PABA; 15% or less; a UVB absorbing organic sunscreen), avobenzone (also called butyl methoxy dibenzoylmethane; 3% or less, a UVA I absorbing organic sunscreen), cinoxate (also called 2-ethoxyethyl p-methoxycinnamate; 3% or less, a UVB absorbing organic sunscreen), dioxybenzone (also called benzophenone-8; 3% or less, a UVB and UVA II absorbing organic sunscreen), homosalate (15% or less, a UVB absorbing organic sunscreen), menthyl anthranilate (also called menthyl 2-aminobenzoate; 5% or less, a UVA II absorbing organic sunscreen), octocrylene (also called 2-ethylhexyl-2-cyano-3,3 diphenylacrylate; 10% or less, a UVB absorbing organic sunscreen), octyl methoxycinnamate (7.5% or less, a UVB absorbing organic sunscreen), octyl salicylate (also called 2-ethylhexyl salicylate; 5% or less, a UVB absorbing organic sunscreen), oxybenzone (also called benzophenone-3; 6% or less, a UVB and UVA II absorbing organic sunscreen), padimate 0 (also called octyl dimethyl PABA; 8% or less, a UVB absorbing organic sunscreen), phenylbenzimidazole sulfonic acid (water soluble; 4% or less, a UVB absorbing organic sunscreen), sulisobenzone (also called benzophenone-4; 10% or less, a UVB and UVA II absorbing organic sunscreen), titanium dioxide (25% or less, an inorganic physical blocker of UVA and UVB), trolamine salicylate (also called triethanolamine salicylate; 12% or less, a UVB absorbing organic sunscreen), and zinc oxide (25% or less, an inorganic physical blocker of UVA and UVB).

For a product marketed in the European Union, preferred cosmetically-acceptable photoactive compounds and concentrations (reported as a percentage by weight of the total cosmetic sunscreen composition, and referring to the final percentage of the sunscreen after addition to the bodywash) include: PABA (5% or less), camphor benzalkonium methosulfate (6% or less), homosalate (10% or less), benzophenone-3 (10% or less), phenylbenzimidazole sulfonic acid (8% or less, expressed as acid), terephthalidene dicamphor sulfonic acid (10% or less, expressed as acid), butyl methoxydibenzoylmethane (5% or less), benzylidene camphor sulfonic acid (6% or less, expressed as acid), octocrylene (10% or less, expressed as acid), polyacrylamidomethyl benzylidene camphor (6% or less), octyl methoxycinnamate (10% or less), PEG-25 PABA (10% or less), isoamyl p-methoxycinnamate (10% or less), ethylhexyl triazone (5% or less), drometrizole trielloxane (15% or less), diethylhexyl butamido triazone (10% or less), 4-methylbenzylidene camphor (4% or less), 3-benzylidene camphor (2% or less), ethylhexyl salicylate (5% or less), ethylhexyl dimethyl PABA (8% or less), benzophenone-4 (5%, expressed as acid), methylene bis-benztriazolyl tetramethylbutylphenol (10% or less), disodium phenyl dibenzimidazole tetrasulfonate (10% or less, expressed as acid), bis-ethylhexyloxyphenol methoxyphenol triazine (10% or less), methylene bisbenzotriazolyl tetramethylbutylphenol (10% or less, also called TINOSORB M), and bisethylhexyloxyphenol methoxyphenyl triazine. (10% or less, also called TINOSORB S).

In some embodiments, the sunscreen additives or sunscreen/bodywashes of the invention include a silicone long-chain molecule with chromophores, e.g., PARASOL SLX (DSM Nutritional Products), which contains benzyl malonate chromophores attached to specific points on a polysiloxane chain. Thus, in some embodiments, the invention provides a sunscreen additive or sunscreen/bodywash composition that contains sunscreen that comprises a silicone long-chain molecule with chromophores. For example, compositions of the invention include a composition containing octyl methoxycinnamate, octocrylene, avobenzone, titanium dioxide, and a silicone long-chain molecule with chromophores. The silicon long-chain molecule may be used in sunscreen additives at about 0.5 to about 5%, or in sunscreen/bodywashes at about 0.2 to about 2%.

Inorganic physical blockers of UVA and UVB useful in the invention further include iron oxide and polymer particles such as those of polyethylene and polyamides.

In some embodiments, the sunscreen additives and sunscreen/bodywashes contain at least one sunscreen active that is cinnamate (e.g., Octylmethoxycinnamate (ethyl hexyl methoxycinnamate), (available under the tradename PARSOL MCX), oxybenzone (e.g., benzophenone-3 (2-Hydroxy-4-Methoxybenzophenone), avobenzone (4-tert-Butyl-4′-methoxydibenzoylmethane or PARSOL 1789), octyl salicylate (2-Ethylhexyl Salicylate), octocrylene (2-Ethylhexyl 2-Cyano-3,3-Diphenylacrylate), methyl anthranilate, and/or titanium dioxide, or combinations thereof.

The sunscreen additives include and a physical blocker sunscreen such as an inorganic or organic compound which may reflect, scatter or absorb light.

Sunscreen additives and sunscreen/bodywashes of the invention may, in some embodiments, contain as a sunscreen component only titanium dioxide. When titanium dioxide is used in compositions of the invention, either alone or in combination with other sunscreens, the titanium dioxide can have an anatase, rutile, or amorphous structure. The titanium dioxide particles can be uncoated or can be coated with a variety of materials including, but not limited to, aluminum compounds such as aluminum oxide, aluminum stearate, aluminum laurate and the like; phospholipids such as lecithin; silicone compounds; and mixtures thereof. Various grades and forms of titanium dioxide are described in CTFA Cosmetic Ingredient Dictionary, 11^{th} Edition (1982), pp. 318-319; U.S. Pat. No. 4,820,508 to Wortzman, issued Apr. 11, 1989; and World Patent No. WO 90/11067 to Elsom et al, published Oct. 4, 1990; these three references are incorporated by reference herein in their entirety. Suitable grades of titanium dioxide for use in the compositions of the present invention are available commercially such as the MT micronized series from Tri-K Industries (Emerson, N.J.). These micronized titanium dioxides generally have a mean primary particle size ranging from about 10 nm to about 50 nm. For example, titanium dioxide having a mean primary particle size of about 15 nm is available under the trade designations MT-150W (uncoated) and MT-100T (coated with stearic acid and aluminum compounds). Uncoated titanium dioxides having mean primary particle sizes of around 35 nm and around 50 nm are available under the trade designations MT-500B and MT-600B, respectively. Other coated titanium dioxides having a mean primary particle size around 15 nm include MT-100F (modified with stearic acid and iron hydroxide) and MT-100S (treated with lauric acid and aluminum hydroxide). Mixtures of two or more types and particle size variations of titanium dioxide can be used in the present invention.

One form of titanium dioxide is silica-coated TiO₂. Such a silica-coated TiO₂ is available under the tradename T-AVO (Eusolex).

If a zinc compound is chosen as the inorganic sunscreen, some zinc-based compositions (e.g., Z-Cote™ HP1 [registered trademark, SkinCeuticals]) provide micro-fine zinc oxide coated with a form of dimethicone. As expressed by the manufacturer, the dimethicone coating transforms the frequently granular and pasty particles of zinc oxide to a smooth formulation which is transparent. The micronizing of these particles achieves the important advantage of providing effective sunscreening without giving the appearance of skin coated with white paint.

Also to be noted in relation to inorganic blockers are Tioveil and Spectraveil (both of the Tioxide Group). Tioveil include products which are 40% dispersions of surface-treated titanium dioxide in a range of cosmetic vehicles. Spectraveil include products which are 60% dispersions of zinc oxide in a range of cosmetic vehicles. In certain variations, these products may be film-formers and may have advantageous uses here.

In sunscreen additives, the total sunscreens comprise about 0.1-50%, or about 1-30%, or about 1-25%, or about 3-25%, or about 5-25%, or about 10-25% or about 15-25%, or about 5, 10, 15, 20, 25, 30, 35, 40, 45, or 50% of the composition (all percentages herein are weight percent unless otherwise specified). In sunscreen/bodywash compositions, the total sunscreens can comprise 0.05-30%, or about 0.5-15%, or about 0.5-12%, or about 1.5-12%, or about 2.5-12%, or about 5-12% or about 7-12%, or about 2.5, 5, 7.5, 10, 12.5, 15, 20, 25, 30, 33, 35, 40, 45, 50, or more than 50% of the composition.

In some embodiments, a sunscreen additive of the invention includes octyl methoxycinnamate at about 4.5-9%, Octocrylene at about 0.5-15%, Avobenzone (e.g., PARSOL 1789) at about 2-4%, and titanium dioxide at about 3-9%. In some embodiments, the octyl methoxy cinnamate is encapsulated, e.g., in amorphous silica. Such encapsulated octyl methoxy cinnamate is commercially available under the trade name UV PEARLS; about 20-40% UV PEARLS supplies about 4.5-9% octyl methoxy cinnamate. In some embodiments, a sunscreen additive of the invention includes octyl methoxycinnamate at about 7.6% (in some embodiments, encapsulated as described, e.g., in UV PEARLS wherein the UV PEARLS are provided at about 33.3%), Octocrylene at about 11.3%, Avobenzone (PARSOL 1789) at about 2.8%, and titanium dioxide at about 6.4%. The sunscreen additives may further include a polyquaternium, e.g., polyquaternium-4. In some embodiments, the polyquaternium-4 is present at about 0.5% to about 5%, in some embodiments, the polyquaternium-4 is present at about 2.8%. The sunscreen additives may further include a film-former, which may comprise dimethicone and/or petrolatum, and/or a preservative, such as BHT. This sunscreen additive may be added to a conventional bodywash formulation (e.g., SUAVE Bodywash) in a ratio of about one part sunscreen additive to two parts bodywash (w/w). Other ratios are encompassed by the invention, e.g., about one part sunscreen additive to about 0.2, 0.5, 0.7 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.2, 3.5, 3.7, 4.0, 4.2, 4.5, 4.7, 5.0, 6.0, 7.0, 8.0, 9.0, 10, 12, 15, or 20 parts bodywash (w/w).

It will be appreciated by those of skill in the art that the various ingredients of the sunscreen additive may be added to the bodywash all at once, or in groups, or separately. In some embodiments, the sunscreen additive comprises at least two components. For example, the first component may comprise all the ingredients except an inorganic or physical blocker sunscreen, and the second component may comprise the inorganic or physical blocker sunscreen. The first component is added to the bodywash with thorough mixing, then the second component is added. For example, in some embodiments, all ingredients except the titanium dioxide are mixed, then added to the bodywash, and then the titanium dioxide is added (see Examples).

In some embodiments, the sunscreen additives of the invention include about 0.1 to 7.5 weight percent of octylmethoxy cinnamate, about 0.1 to 6 parts weight percent of octyl salicylate, about 0.1 to 5 parts weight percent of oxybenzone, about 1 to 10 weight percent of cationic surfactant, and about 0.01 to 1 weight percent of a quaternized compound. These composition may further include a film former. These compositions may further include 0.01 to 1 weight percent of a preservative.

This UV component additives are not be limited to the commonly categorized sunscreens but to all suitable compounds including polymers or other compositions that exhibit sunscreen properties described above.

B. Non-Sunscreen Additives and Actives

In one aspect, the invention provides additives containing non-sunscreen active ingredients, where the additive is designed to be added to a composition for applications to a variety of surfaces in, for example, agricultural, textile, industrial, transportation, marine, pharmaceutical, or personal care applications. One aspect comprises a topical application, e.g., a bodywash. These actives may be used in combination with the sunscreens described above in a sunscreen additive or sunscreen/bodywash, or may be used in separate, non-sunscreen compositions. In some embodiments, at least one of the additives is encapsulated. In another aspect, the invention provides a composition for topical application, e.g., a bodywash, containing one or more such additives. These actives may be used in combination with the sunscreens described above in a sunscreen additive or sunscreen/bodywash, or may be used in separate, non-sunscreen compositions.

Non-limiting examples of non-sunscreen actives useful in compositions of the invention include sunless tanning actives, skin lightening actives, anti-acne actives, anti-skin wrinkling and anti-skin aging actives, vitamins, anti-inflammatory actives, anesthetic actives, analgesic actives, anti-pruritic actives, anti-microbial actives (e.g. antifungals, antibacterials, and antiparasitics), anti-virals, anti-allergenics, medicinal actives (e.g., skin rash, skin disease and dermatitis medications), anti-cellulite additives, insect repellant actives, antioxidants, hair growth promoters, hair growth inhibitors, hair bleaching agents, deodorant compounds, fragrances, pharmaceuticals, moisturizers, dental care agents, personal care agents, nutraceuticals, and mixtures and combinations thereof.

In some embodiments, the actives can also include constituents for gene therapy including vectors such including viral and non-viral vectors. Viral vectors include, for example, adenoviruses, adeno-associated viruses, and retroviruse). The gene therapy constituents can include nucleic acids such as DNA or RNA in the form of plasmid DNA, and single or double stranded oligonucleotides. The nucleic acids can be included, for example, within liposomes, virosomes, and dendrimers.

The non-sunscreen additives can be useful for the textiles, comprising, for example, smoothing agents and softeners, anti-setting treatment of wool, antistatic agents, binders and auxiliaries for pigment dyeing, catalysts, crosslinking agents, filling and stiffening agents, hydrophilizing agents, non-felt finish on wool, water-repellents, wetting and antifoaming agents, sizes, textile waxes, activators for peroxide bleaching, complexing agents, extracting agents, peroxide killer, pretreatment agents for printing on wool, reduction bleaching agents, and special extracting agents

The additives can be used to improve lubricity or friction, wetability, water absorption, water release, fluid release, surface energy, surface area, visibility, compatibility, leaching, intended release of a substances, biostatic behavior, chemical reactivity, interaction with proteins and other molecules, adhesion or repellence of microorganisms or marine life, incrustation, sedimentation, calcification, antigenicity and biocompatibility.

The additives can include antifouling agents including marine antifouling agents such as algaecides and molluscicides. The actives can provide marine antifouling activity including both the elimination of and inhibition of growth of marine organisms. Marine organisms controlled by marine antifouling agents suitable for use in this invention include both hard and soft fouling organisms. Generally speaking, the term “soft fouling organisms” refers to plants and invertebrates, such as slime, algae, kelp, soft corals, tunicates, hydroids, sponges, and anemones, while the term “hard fouling organisms” refers to invertebrates having some type of hard outer shell, such as barnacles, tubeworms, and molluscs.

The additives can be used for agricultural applications including agents to improve plant growth, nutrients, fertilizers, hygroscopic agents, and pesticides. Agricultural pesticides include agricultural fungicides, herbicides, insecticides and miticides. An agricultural fungicide generally refers to a compound capable of inhibiting the growth of or controlling the growth of fungi in an agricultural application, such as treatment of plants and soil; “herbicide” refers to a compound capable of inhibiting the growth of or controlling the growth of certain plants; “insecticide” refers to a compound capable of controlling insects; and “miticide” refers to a compound capable of controlling mites. Additives for agricultural applications include either topical applications such as leaf, stem, root, or trunk of trees and or applications surrounding plants or trees for uptake. Applications can also include addition to algae, fungi, bacteria, viruses or parasites on any substrate or in any environment these organisms are found.

Sunless tanning actives include dihydroxyacetone (DHA); glyceryl aldehyde; tyrosine and tyrosine derivatives such as malyltyrosine, tyrosine glucosinate, and ethyl tyrosine; phospho-DOPA, indoles and derivatives; and mixtures thereof.

Non-limiting examples of skin lightening actives include EMBLICA (also an antioxidant), monobenzone (a depigmenting agent), kojic acid, arbutin, ascorbic acid and derivatives thereof (e.g., magnesium ascorbyl phosphate or sodium ascorbyl phosphate), and extracts (e.g., mulberry extract, placental extract). Non-limiting examples of skin lightening agents suitable for use herein also include those described in WO 95/34280, WO 95/07432, and WO 95/23780.

Vitamins may be included in the compositions of the present invention. Examples include Vitamin A and derivatives thereof (including, for example, retinol, see anti-wrinkling actives), ascorbic acid (Vitamin C and derivatives), Vitamin B (e.g., riboflavin, vitamin B₂), biotin, Vitamin D (all forms), Vitamin E and derivatives thereof such as tocopheryl acetate, beta-carotene, panthothenic acid and mixtures thereof.

Anti-acne actives include benzoyl peroxide, erythromycin, clindamycin phosphate, 5,7-dichloro-8-hydroxyquinoline, resorcinol, resorcinol acetate, salicylic acid, azaleic acid, long chain dicarboxylic acids, sulfur, zinc, various natural agents such as those derived from green tea, and mixtures thereof. Other non-limiting examples of suitable anti-acne actives for use herein are described in U.S. Pat. No. 5,607,980, which description is incorporated herein by reference.

Anti-skin wrinkling actives include a variety of agents, often in combination, that prevent or treat wrinkling through a variety of actions. Many approaches are taken to reduce the appearance of facial wrinkles based on the understanding of the molecular basis of wrinkle formation. Such treatments include cosmetic products, drug therapy and surgical procedures. For example, many cosmetic products contain hydroxy acids, which may stimulate collagen synthesis. Another common treatment utilizes retinol, retinoic, retinol palmitate, a derivative of vitamin A, (or its stronger, prescribed version Retin-A and Renova) which helps collagen production. Bicyclic aromatic compounds with retinoid-type activity, which are useful in particular in preventing or treating various keratinization disorders, are described in EP 679 630. These compounds are particularly active for repairing or combating chronological or actinic ageing of the skin, for example such as in anti-wrinkle products. Antioxidants such as vitamin C and E and coenzyme Q-10 are believed to counteract free radicals, which damage cells and cause aging and have been used in treatments of wrinkles. For instance, the FDA has approved cosmetic use of Botox (an extremely diluted form of botulinum toxin) to treat glabella frown lines. Thus non-sunscreen actives of the invention that are anti-skin aging or anti-wrinkling actives may contain, alone or in combination, the bicyclic aromatic compounds defined above, other compounds which have retinoid-type activity, free-radical scavengers, hydroxy or keto acids or derivatives thereof. The term “free-radical scavenger” refers to, for example, α-tocopherol, superoxide dismutase, ubiquinol or certain metal-chelating agents. Hydroxy acids include, e.g., alpha-hydroxy acids such as lactic acid and glycolic acid or beta-hydroxy acids such as salicylic acid and salicylic acid derivatives such as the octanoyl derivative; other hydroxy acids and keto acids include malic, citric, mandelic, tartaric or glyceric acids or the salts, amides or esters thereof.

Other anti-wrinkling agents and anti-skin aging agents useful in the invention include sulfur-containing D and L amino acids and their derivatives and salts, particularly the N-acetyl derivatives, an example of which is N-acetyl-L-cysteine; thiols, e.g. ethane thiol; fat-soluble vitamins, ascorbyl palmitate, ceramides, pseudoceramides (e.g., pseudoceramides described in U.S. Pat. Nos. 5,198,210; 4,778,823; 4,985,547; 5,175,321, all of which are incorporated by reference herein), phospholipids (e.g., distearoyl lecithin phospholipid), fatty acids, fatty alcohols, cholesterol, plant sterols, phytic acid, lipoic acid; lysophosphatidic acid, and skin peel agents (e.g., phenol and the like), and mixtures thereof. In some embodiments, the fatty acids or alcohols are those that have straight or branched alkyl chains containing 12-20 carbon atoms. In one embodiment, the fatty acid is linoleic acid since linoleic acid assists in the absorption of ultraviolet light and furthermore is a vital component of the natural skin lipids. Other non-limiting examples of suitable anti-wrinkle actives for use herein are described in U.S. Pat. No. 6,217,888, which description is incorporated herein by reference.

Anti-inflammatory actives include steroidal, non-steroidal, and other compounds.

Non-limiting examples of steroidal anti-inflammatory agents suitable for use herein include corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof may be used. One steroidal anti-inflammatory for use is hydrocortisone.

Nonsteroidal anti-inflammatory agents are also suitable for use herein as skin active agents in the compositions of the invention. Non-limiting examples of non-steroidal anti-inflammatory agents suitable for use herein include oxicams (e.g., piroxicam, isoxicam, tenoxicam, sudoxicam, CP-14,304); salicylates (e.g., aspirin, disalcid, benorylate, trilisate, safapryn, solprin, diflunisal, fendosal); acetic acid derivatives (e.g., diclofenac, fenclofenac, indomethacin, sulindac, tolmetin, isoxepac, furofenac, tiopinac, zidometacin, acematacin, fentiazac, zomepirac, clindanac, oxepinac, felbinac, ketorolac); fenamates (e.g., mefenamic, meclofenamic, flufenamic, niflumic, tolfenamic acids); propionic acid derivatives (e,g., ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, tiaprofenic); pyrazoles (e.g., phenylbutazone, oxyphenbutazone, feprazone, azapropazone, trimethazone); and combinations thereof as well as any dermatologically acceptable salts or esters of thereof. COX-2 inhibitors are also suitable for use herein, and include, but are not limited to, AZD 3582 (ASTRAZENECA and NicOx), Celecoxib (PHARMACIA Corp.) (4-[5-(4-methylphenyl)-3-(trifluoromethyl)-1H-pyrazol-1-yl]benzenesulfonamide), Meloxicam (BOEHRINGER INGELHEIM Pharmaceuticals) (4-hydroxy-2-methyl-N-(5-methyl-2-thiazolyl)-2H-1,2GW-406381 (GLAXOSMITHKLINE), Etoricoxib (MERCK & Co.), Rofecoxib (MERCK & Co.) (4-[4-(methylsulfonyl)phenyl]-3-phenyl-2(5H)-furanone), Lumiracoxib (NOVARTIS Pharma AG), Valdecoxib (PHARMACIA Corp.) (4-(5-methyl-3-phenyl-4-isox-azolyl) benzenesulfonamide), and Etodolac (WYETH Ayerst Laboratories) ((.+-.) 1,8-diethyl-1,3,4,9-tetrahydropyrano-[3,4-b]acid).

Other non-limiting examples of suitable anti-inflammatory or similar other skin active agents include candelilla wax, bisabolol (e.g., alpha bisabolol), aloe vera, plant sterols (e.g., phytosterol), Manjistha (extracted from plants in the genus Rubia, particularly Rubia cordifolia), and Guggal (extracted from plants in the genus Commiphora, particularly Commiphora mukul), kola extract, chamomile, red clover extract, sea whip extract, anise oil, garlic oil, ginger extract, vasoconstrictors such as phenylephrine hydrochloride, and combinations thereof.

Further non-limiting examples of suitable anti-inflammatory or similar other skin active agents include compounds of the Licorice (the plant genus/species Glycyrrhiza glabra) family, including glycyrrhetic acid, glycyrrhizic acid, and derivatives thereof (e.g., salts and esters). Suitable salts of the foregoing compounds include metal and ammonium salts. Suitable esters include C₂-C₂₄ saturated or unsaturated esters of the acids, C₁₀-C₂₄, or C₁₆-C₂₄. Specific non-limiting examples of the foregoing include oil soluble licorice extract, the glycyrrhizic and glycyrrhetic acids themselves, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid, disodium 3-succinyloxy-beta-glycyrrhetinate, and combinations thereof.

Anesthetic actives include butamben picrate, lidocaine, xylocalne, benzocaine, bupivacaine, chlorprocaine, dibucaine, etidocaine, mepivacaine, tetracaine, dyclonine, hexylcaine, procaine, cocaine, ketamine, pramoxine, phenol, and pharmaceutically acceptable salts thereof.

Analgesic actives include dyclonine hydrochloride, aloe vera, fentanyl, capsaicin, and the like.

Anti-pruritic actives include alclometasone dipropionate, betamethasone valerate, and isopropyl myristate MSD.

Anti-microbial actives include antifungal, antibacterial, and antiseptic compounds. Antifungal compounds include, but are not limited to, compounds such as imidazole antifungals. Specific antifungals include butocouazole nitrate, miconazole, econazole, ketoconazole, oxiconizole, haloprogin, clotrimazole, and butenafine HCl, naftifine, terbinafine, ciclopirox, and tolnaftate. Antibacterial and antiseptic compounds include phenol-TEA complex, mupirocin, triclosan, chlorocresol, chlorbutol, iodine, clindamycin, CAE (Anjinomoto Co., Inc., containing DL-pyrrolidone Carboxylic acid salt of L-Cocoyl Arginine Ethyl Ester), povidone-iodine, polymyxin b sulfate-bacitracin, zinc-neomycin sulfate-hydrocortisone, chloramphenicol, methylbenzethonium chloride, and erythromycin and antiseptics (e.g., benzalkonium chloride, benzethonium chloride, chlorhexidine gluconate, mafenide acetate, nitrofurazone, nitromersol and the like may be included in compositions of the invention. Many deodorant compounds are also antimicrobial (see below). Antiparasitics, such as lindane may also be included.

Further examples of antimicrobial and antifungal actives useful in the compositions of the present invention include, but are not limited to, P-lactam drugs, quinolone drugs, ciprofloxacin, norfloxacin, tetracycline, amikacin, 2,4,4′-trichloro-2′-hydroxy diphenyl ether, 3,4,4′-trichlorocarbanilide, phenoxyethanol, phenoxy propanol, phenoxyisopropanol, doxycycline, capreomycin, chlorhexidine, chlortetracycline, oxytetracycline, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, amanfadine sulfate, octopirox, parachlorometa xylenol, nystatin, tolnaftate, zinc pyrithione and clotrimazole

Compositions of the invention may include antiviral agents. Suitable anti-viral agents include, but are not limited to, metal salts (e.g., silver nitrate, copper sulfate, iron chloride, etc.) and organic acids (e.g., malic acid, salicylic acid, succinic acid, benzoic acid, etc.). In particular compositions which contain additional suitable anti-viral agents include those described in copending U.S. patent application Ser. Nos. 09/421,084 (Beerse et al.); 09/421,131 (Biedermann et al.); 09/420,646 (Morgan et al.); and 09/421,179 (Page et al.), which were each filed on Oct. 19, 1999

Anti-allergenics include antihistamines. Antihistamines can be of H₁ or H₂ antagonists or other types of histamine release inhibitors. The H₁ antagonists can be sedating or non-sedating. Examples of H₁-sedating antihistamines include diphenhydramine (Benadryl), chlorpheniramine, tripelennamine, promethazine, clemastine, doxylamine, benadryl etc. Examples of H₁-non-sedating antihistamines include astemizole, terfenadine, loratadine etc. Examples of H₂ antagonists include cimetadine, famotidine, nizatidine, and ranitidine. Examples of histamine-release-inhibitors include cromolyn.

A further active useful in the invention may be a medicinal for treatment of dermatological conditions such as psoriasis, acne, eczema, and other skin conditions due to disease, pathology, accident, and the like. Medicinals include burn relief ointments, such as o-amino-p-toluenesulfonamide monoacetate; dermatitis relief agents, such as the active steroid amcinonide, diflorasone diacetate, and hydrocortisone; diaper rash relief agents, such as methylbenzethonium chloride and the like; herpes treatment drugs, such as O—[(2-hydroxyethoxy)methyl]guanine; psoriasis, seborrhea and scabicide agents, such as shale oil and derivatives thereof, elubiol, ketoconazole, coal tar and petroleum distillates, salicylic acid, zinc pyrithione, selenium sulfide, hydrocortisone, sulfur, menthol, psoralen, pramoxine hydrochloride anthralin, and methoxsalen; steroids, such as 2-(acetyloxy)-9-fluoro-1′,2′,3′,4′-tetrahydro-11-hydroxypregna-1,4-dieno[16,17-b]naphthalene-3,20-dione and 21-chloro-9-fluoro-1′,2′,3′,4′-tetrahydro-11b-hydroxypregna-1,4-dieno[16z,17-b]naphthalene-3,20-dione, and others including those that are antiinflammatories. Other medicinals include those useful in the treatment of exposure to poison oak, poison ivy, poison sumac, and the like. These include camphor, menthol, benzocaine, butamben picrate, dibucaine, dibucaine hydrochloride, dimethisoquin hydrochloride, dyclonine hydrochloride, lidocaine, metacresol, lidocaine hydrochloride, pramoxine hydrochloride, tetracaine, tetracaine hydrochloride, benzyl alcohol, camphorated metacresol, juniper tar, phenol, phenolate sodium, resorcinol, diphenhydramine hydrochloride, tripelennamine hydrochloride, hydrocortisone, a corticosteroid, and hydrocortisone acetate. Any other medication capable of topical administration also can be incorporated in a composition of the present invention in an amount sufficient to perform its intended function.

Anticellulite actives include isobutylmethylxanthine, caffeine, theophylline, theobromine, aminophylline, yohimbine, and mixtures thereof.

Examples of actives suitable for treating hair loss include, but are not limited to potassium channel openers or peripheral vasodilators such as minoxidil, diazoxide, and compounds such as N*-cyano-N-(tert-pentyl)-N′-3-pyridinyl-guanidine (“P-1075”) as disclosed in U.S. Pat. No. 5,244,664, which is incorporated herein by reference; vitamins, such as vitamin E and vitamin C, and derivatives thereof such as vitamin E acetate and vitamin C palmitate; hormones, such as erythropoietin, prostaglandins, such as prostaglandin EI and prostaglandin F2-alpha; fatty acids, such as oleic acid; diuretics such as spironolactone; heat shock proteins (“HSP”), such as HSP 27 and HSP 72; calcium channel blockers, such as verapamil HCL, nifedipine, and diltiazemamiloride; immunosuppressant drugs, such as cyclosporin and Fk-506; 5 alpha-reductase inhibitors such as finasteride; growth factors such as, EGF, IGF and FGF; transforming growth factor beta; tumor necrosis factor; non-steroidal anti-inflammatory agents such as benoxaprofen; retinoids such as tretinoin; cytokines, such as IL-6, IL-1 alpha, and IL-1 beta; cell adhesion molecules such as ICAM; glucorcorticoids such as betametasone; botanical extracts such as aloe, clove, ginseng, rehmannia, swertia, sweet orange, zanthoxylum, Serenoa repens (saw palmetto), Hypoxis rooperi, stinging nettle, pumpkin seeds, and rye pollen; other botanical extracts including sandlewood, red beet root, chrysanthemum, rosemary, burdock root and other hair growth promoter activators which are disclosed in DE 4330597 which is incorporated by reference in its entirety herein; homeopathic agents such as Kalium Phosphoricum D2, Azadirachta indica D2, and Joborandi DI; genes for cytokines, growth factors, and male-pattered baldness; antifungals such as ketoconazole and elubiol; antibiotics such as streptomycin; proteins inhibitors such as cycloheximide; acetazolamide; benoxaprofen; cortisone; diltiazem; hexachlorobenzene; hydantoin; nifedipine; penicillamine; phenothaiazines; pinacidil; psoralens, verapamil; zidovudine; alpha-glucosylated rutin having at least one of the following rutins: quercetin, isoquercitrin, hespeddin, naringin, and methylhesperidin, and flavonoids and transglycosidated derivatives thereof which are all disclosed in JP 7002677, which is incorporated by reference in its entirety herein; and mixtures thereof. In some embodiments, the hair loss treatment agents include minoxidil, 6-(1-piperidinyl)-2,4-pyrimidinediamine-3-oxide, N′-cyano-N-(tert-pentyl)-N′-3-pyridinyl-guanidine, finasteride, retinoids and derivatives thereof, ketoconazole, elubiol or mixtures thereof.

Examples of actives suitable for use in inhibiting hair growth include: serine proteases such as trypsin; vitamins such as alpha-tocophenol (vitamin E) and derivatives thereof such as tocophenol acetate and tocophenol palmitate; antineoplastic agents, such as doxorubicin, cyclophosphamide, chlormethine, methotrexate, fluorouracil, vincristine, daunorubicin, bleomycin and hydroxycarbamide; anticoagulants, such as heparin, heparinoids, coumaerins, detran and indandiones; antithyroid drugs, such as iodine, thiouracils and carbimazole; lithium and lithium carbonate; interferons, such as interferon alpha, interferon alpha-2a and interferon alpha-2b; retinoids, such as retinol (vitamin A), isotretinoin: glucocorticoids such as betamethasone, and dexamethosone; antihyperlipidaemic drugs, such as triparanol and clofibrate; thallium; mercury; albendazole; allopurinol; amiodarone; amphetamines; androgens; bromocriptine; butyrophenones; carbamazepine; cholestyramine; cimetidine; clofibrate; danazol; desipramine; dixyrazine; ethambutol; etionamide; fluoxetine; gentamicin, gold salts; hydantoins; ibuprofen; impramine; immunoglobulins; indandiones; indomethacin; intraconazole; levadopa; maprotiline; methysergide; metoprolol; metyrapone; nadolol; nicotinic acid; potassium thiocyanate; propranolol; pyridostimine; salicylates; sulfasalazine; terfenadine; thiamphenicol; thiouracils; trimethadione; troparanol; valproic acid; and mixtures thereof. In some embodiments, the hair growth inhibitory agents include serine proteases, retinol, isotretinoin, betamethoisone, alpha-tocophenol and derivatives thereof, or mixtures thereof.

Examples of hair bleaching agents include perborate or persulfate salts.

Deodorant compounds include astringent salts and bioactive compounds. The astringent salts include organic and inorganic salts of aluminum, zirconium, zinc, and mixtures thereof. The anion of the astringent salt can be, for example, sulfate, chloride, chlorohydroxide, alum, formate, lactate, benzyl sulfonate or phenyl sulfonate. Exemplary classes of antiperspirant astringent salts include aluminum halides, aluminum hydroxyhalides, zirconyl oxyhalides, zirconyl hydroxyhalides, and mixtures thereof. Exemplary aluminum salts include aluminum chloride and the aluminum hydroxyhalides having the general formula Al₂(OH)ₓQ_{y}XH₂O, wherein Q is chlorine, bromine or iodine; x is about 2 to about 5; x+y is about 6, wherein x and y are not necessarily integers; and X is about 1 to about 6. Exemplary zirconium compounds include zirconium oxy salts and zirconium hydroxy salts, also referred to as zirconyl salts and zirconyl hydroxy salts, and represented by the general empirical formula ZrO(OH)₂-nz L_{z}, wherein z varies from about 0.9 to about 2 and is not necessarily an integer; n is the valence of L; 2-nz is greater than or equal to 0; and L is selected from the group consisting of halides, nitrate, sulfamate, sulfate, and mixtures thereof. In some cases, the active ingredients constitute reodorant compounds.

Exemplary deodorant compounds therefore include, but are not limited to, aluminum bromohydrate, potassium alum, sodium aluminum chlorohydroxy lactate, aluminum sulfate, aluminum chlorohydrate, aluminum-zirconium tetrachlorohydrate, an aluminum-zirconium polychlorohydrate complexed with glycine, aluminum-zirconium trichlorohydrate, aluminum-zirconium octachlorohydrate, aluminum sesquichlorohydrate, aluminum sesquichlorohydrex PG, aluminum chlorohydrex PEG, aluminum zirconium octachlorohydrex glycine complex, aluminum zirconium pentachlorohydrex glycine complex, aluminum zirconium tetrachlorohydrex glycine complex, aluminum zirconium trichlorohydrex glycine complex, aluminum chlorohydrex PG, zirconium chlorohydrate, aluminum dichlorohydrate, aluminum dichlorohydrex PEG, aluminum dichlorohydrex PG, aluminum sesquichlorohydrex PG, aluminum chloride, aluminum zirconium pentachlorohydrate, numerous other useful antiperspirant compounds listed in the CTFA Handbook at p. 56, incorporated herein by reference, and mixtures thereof.

In addition to the astringent salts, the deodorant compound can be a bacteriostatic quaternary ammonium compound, such as, for example, cetyl trimethyl ammonium bromide, cetyl pyridinium chloride, benzethonium chloride, diisobutylbenzoxyethoxyethyldimethylbenzyl ammonium chloride, sodium N-lauryl sarcosine, sodium N-polymethyl sarcosine, lauroyl sarcosine, N-myristolyl glycine, potassium N-lauroyl sarcosine, and stearyl trimethyl ammonium chloride; or a bioactive compound; or a carbonate or bicarbonate salt, such as, for example, the alkali metal carbonates and bicarbonates, and the ammonium and tetralkylammonium carbonates and bicarbonates. Other useful deodorant compounds include chlorophyllin copper complex, aluminum chloride, aluminum chloride hexahydrate, and methylbenzethonium chloride.

Antioxidants are also useful in formulations of the invention. Typical suitable antioxidants include propyl, octyl and dodecyl esters of gallic acid, butylated hydroxyanisole (BHA, usually purchased as a mixture of ortho and meta isomers), butylated hydroxytoluene (BHT), nordihydroguaiaretic acid, Vitamin A, ascorbic acid and its salts, ascorbyl esters of fatty acids, ascorbic acid derivatives (e.g., magnesium ascorbyl phosphate, sodium ascorbyl phosphate, ascorbyl sorbate), tocopherol, tocopherol acetate, other esters of tocopherol, tocotrienols and their esters, and 6-hydroxy-2,5,7,8-tetramethylchroman-2-carboxylic acid (commercially available under the tradename TROLOX). Other suitable antioxidants include uric acid and its salts and alkyl esters, sorbic acid and its salts, lipoic acid, amines (e.g., N,N-diethylhydroxylamine, amino-guanidine), sulfhydryl compounds (e.g., glutathione, N-acetyl cysteine), dihydroxy fumaric acid and its salts, lycine pidolate, arginine pilolate, nordihydroguaiaretic acid, bioflavonoids, curcumin, lysine, methionine, proline, superoxide dismutase, silymarin, tea extracts, grape skin/seed extracts, melanin, and rosemary extracts may be used. It is often desired that the antioxidants be photostable antioxidants. An exemplary photostable antioxidant is marketed under the tradename EMBLICA by EMD Chemicals. See, e.g., U.S. Pat. No. 6,831,191. Antioxidants (e.g., EMBLICA), may be included in sunscreen additives at about 0.05 to about 5%, or about 0.05 to about 2%, or about 0.1%, 0.2%, 0.3%, or 0.4%, or in sunscreen/bodywashes at about 0.02 to about 2%, or about 0.02 to about 1%, or about 0.04%, 0.06%, 0.08%, 0.1%, 0.2%, or 0.3%.

Insect repellants include the most widely used active agent for personal care products, N,N-Diethyl-m-toluamide, frequently called “DEET” and available in the form of a concentrate containing at least about 95 percent DEET. Other synthetic chemical repellents include dimethyl phthalate, ethyl hexanediol, indalone, di-n-propylisocinchoronat-e, bicycloheptene, dicarboximide and tetrahydrofuraldehyde. Certain plant-derived materials also have insect repellent activity, including citronella oil and other sources of citronella (including lemon grass oil), limonene, rosemary oil and eucalyptus oil. Choice of an insect repellent for incorporation into compositions of the invention will frequently be influenced by the odor of the repellent. The amount of repellent agent used will depend upon the choice of agent; DEET is useful at high concentrations, such as up to about 15 percent or more, while some of the plant-derived substances are typically used in much lower amounts, such as 0.1 percent or less. Fragrances include essential oils, natural derivatives, and water soluble frangraces. J. Lawless, The Illustrated Encyclopedia of Essential Oils (1995), Element Books, USA, pp. 36-41, 50-55, 57-58, 62, 108, 156-157, 160, 194-195, 204, 214, and 234. Non-limiting examples of essential oils are cedarwood oil, eucalyptus oil, patchouli oil, sandalwood oil, vetiver oil, guaiacwood oil, bay oil, clove oil, chamomile oil, ginger oil, cumin oil, pepper oil, rosemary oil, hinoki oil, hiba oil, pimentoberry resinoid and myrrh resinoid.

A nutraceutical is a substance that is a food or a part of a food and provides medical or health benefits, including the prevention and treatment of disease. Such substances may be isolated nutrients, dietary supplements, genetically engineered designer foods, herbal products.

A pharmaceutical as used herein is a compound that has medicinal or healing properties. The pharmaceuticals useful as actives of the present invention include the topically active compounds such as anti-inflammatory agents, anti-acne agents, and medicinals are pharmaceutical compounds described above, and also include compounds with medicinal or healing properties that are not topically active.

The compositions of the present invention may contain a wide range of additional active components. The CTFA Cosmetic Ingredient Handbook, Seventh Edition, 1997 and the Eighth Edition, 2000, which are incorporated by reference herein in its entirety, describes a wide variety of active ingredients commonly used in skin care compositions, which are suitable for use in the compositions of the present invention. Other topically-active compounds are listed in Remington's Pharmaceutical Sciences, 20th Ed., Lippincott Williams & Witkins, Baltimore, Md. (2000) (hereinafter Remington's), U.S. Pharmacopeia and National Formulary, The United States Pharmacopeial Convention, Inc., Rockville, Md. and Physician's Desk Reference, Medical Economics Co., Inc., Oradell, N.J. incorporated herein by reference.

The non-sunscreen active may be provided as is or in encapsulated form. Besides the encapsulated active, in some embodiments an additive or composition for topical application containing the active further includes a cationic polymer, as described herein, as well as, optionally, a film former, a preservative, and/or an antioxidant that is stable upon exposure to sunlight. Other components may be as described herein. In some embodiments the additive or composition for topical application may comprise two, three, four, five, six, seven, eight, nine, ten, or more than ten actives, each of which may be encapsulated or non-encapsulated, in any combination.

C. Encapsulation

The actives used in the invention may be encapsulated. Any means of encapsulation known in the art, including but not limited to liposomes, maltodextrin capsules, silica gels, siloxanes, and the like, may be used in the compositions of the invention. The actives of the invention can, for example, be encapsulated within microcapsules. Microcapsules can be viewed as having two parts, the core and the shell. The core contains the active ingredient, while the shell surrounds and protects the core. The core materials used in the invention can be solid or liquid, and if liquid, can be, for example, in the form of a pure compound, solution, dispersion or emulsion. The shell material can be a natural or synthetic polymer material or can be an inorganic material, such as a silica-based shell. The shell can be made permeable, semi-permeable or impermeable. Permeable and semi-permeable shells can be used for release applications. Semi-permeable capsules can be made to be impermeable to the core material but permeable to low molecular-weight liquids and can be used to absorb substances from the environment and to release them again when brought into another medium. The impermeable shell encloses the core material. To release the content of the core material the shell must be ruptured. Microencapsulation useful in the present invention is described, for example, in Ghosh, K., Functional Coatings and Microencapsulation: A General Perspective, Wiley-VCH, Weinheim, 2006, Benita, S., Microencapsulation: Methods and Industrial applications, Marcel Dekker, Inc., NY, 1996., and Arshady, R., Microspheres, Microcapsules and Liposomes, Citrus Books, London, 1999.

The present invention can also incorporate mesopourous shells. The synthesis of mesoporous hollow spheres is described in Yeh et al., Langmuir, 2006, 22, 6, and in U.S. Pat. No. 6,913,825.

The encapsulated actives of the present invention can be made by chemical, phisico-chemical, and physico-mechanical methods such as suspension, dispersion and emulsion, coacervation, layer-by-layer polymerization (L-B-L) assembly, sol-gel encapsulation, supercritical CO2-assisted microencapsulation, spray-drying, multiple nozzle spraying, fluid-bed coating, polycondensation, centrifugal techniques, vacuum encapsulation, and electrostatic encapsulation.

In some embodiments, the active is encapsulated sol-gel microcapsules, such as silica sol-gel microcapsules. Such microcapsules are described in U.S. Pat. Nos. 6,238,650; 6,436,375, 6,303,149; 6,468,509, and in U.S. Patent Application No. 2005/0123611. Thus, in some embodiments the invention provides an additive for addition to a composition for topical application, where the additive comprises an encapsulated sunscreen active, and optionally further comprises a cationic polymer. In other embodiments the invention provides a composition for topical application that contains an additive, where the additive comprises an encapsulated non-sunscreen active, and optionally further comprises a cationic polymer. Further ingredients include film formers, antioxidants, preservatives, and other ingredients as listed herein. The composition for topical application may be, e.g., a bodywash.

The sol-gel process can produce particles with a ceramic shell. The shells are prepared by a sol-gel based process in which partly hydrolyzed oxides of suitable metals are prepared in the presence of an active material by hydrolysis of the gel precursor followed by condensation (alternatively referred to as polycondensation). The gel precursor may be, for example, a metal oxide gel precursor including silicon oxide gel precursor or a transition metal oxide precursor. The type of gel precursor used will depend on the intended use of the ceramic particles. The gel precursor is typically a silica-based gel precursor, an alumina-based gel precursor, a titanium dioxide-based gel precursor, an iron oxide based gel precursor, a zirconium dioxide-based gel precursor or any combination thereof. A functionalized, derivatized or partially hydrolyzed gel precursor may also be used.

There are many silicon precursors which can used in the present invention. For convenience, they can be divided into 4 categories, the silicates (silicon acetate, silicic acid or salts thereof) the silsequioxanes and poly-silsequioxanes, the silicon alkoxides (e.g. from silicon methoxide to silicon octadecyloxide), and functionalised alkoxides for ORMOCER (Organically Modified Ceramics) production (such as ethyltrimethoxysilane, aminopropyltriethoxysilane, vinyltrimethoxysilane, diethyldiethoxysilane, diphenyldiethoxysilane, etc). Further specific examples of silica-based gel precursors include tetramethoxysilane (TMOS), tetraethoxysilane (TEOS), tetrabutoxysilane (TBOS), tetrapropoxysilane (TPOS), polydiethoxysilane, methyltrimethoxysilane, methyltriethoxysilane, ethyltriethoxysilane, octylpolysilsesquioxane and hexylpolysilsesquioxane. In some embodiments, the silica based precursors of the present invention are TEOS and TMOS.

Non-limiting examples of alumina-based gel precursors include aluminium ethoxide, aluminium n- or iso-propoxide, aluminium n- or sec- or tert-butoxide. The alkoxide can also be modified using carboxylic acids (for example, acetic, methacrylic, 2-ethylhexanoic acid) or beta di-ketones such as acetylacetone, ethyl-acetylacetone, benzoylacetone, or other complexing agent.

Non-limiting examples of titanium or zirconium gel precursors include the alkoxides (e.g. ethoxide, propoxide, butoxide), the metal salts (e.g. chloride, oxychloride, sulfate, nitrate) and the acid and beta diketone complexes.

The silica gel precursor or the metal oxide gel precursor may include, for example, from one to four alkoxide groups each having from 1 or more oxygen atoms, and from 1 to 18 carbon atoms, more typically from 1 to 5 carbon atoms. The alkoxide groups may be replaced by one or more suitable modifying groups or functionalized or derivatized by one or more suitable derivatizing groups (see K. Tsuru et al., J. Material Sci. Mater. Medicine, 1997, 8).

Typically, the silica gel precursor is a silicon alkoxide or a silicon alkyl alkoxide.

Particular examples of suitable silicon alkoxide precursors include such as methoxide, ethoxide, iso-propoxide, butoxide and pentyl oxide. Particular examples of suitable silicon or metal alkyl (or phenyl) alkoxide precursors include methyl trimethoxysilane, di-methyldimethoxysilane, ethyltriethoxysilane, diethyldiethoxysilane, triethyl-methoxysilane, phenyltriethoxysilane, diphenyldiethoxysilane, vinyltriethoxysilane, etc. Alternatively, the silica gel precursor may be a silicon carboxylate. For example, an acetate, tartrate, oxalate, lactate, propylate, formate, or citrate. Examples of other functional groups attached to silica gel precursors include esters, alkylamines and amides.

Typically, the metal oxide gel precursor is a metal alkoxide which may be derivatised or functionalised. Examples of suitable metal oxide precursors include alkoxides such as methoxide, ethoxide, iso-propoxide, butyloxide and pentyl oxide. Alternatively, metal oxide gel precursor may be a metal carboxylate or a metal beta-diketonate, for example, an acetate, tartrate, oxalate, lactate, propylate, formate, citrate, or acetylacetonate. Examples of other functional groups attached to metal oxide precursors include esters, alkylamines and amides. More than one type of metal ion may be present.

Sol-gel processing is based on the hydrolysis and condensation of appropriate precursors. Water is thus typically used as the condensing agent.

The sol-gel process is carried out in the presence of a surfactant. Suitable surfactants may have a hydrophilic head group and a hydrophyllic tail group. Non-limiting examples of hydrophyllic head groups are sorbitan, polyether, polyoxyethylene, sulfosuccinate, phosphate, carboxylate, sulfate, amino or acetylacetonate and a hydrophobic tail group. The tail group may be, for example, straight or branched chain hydrocarbons with from about 8 to 24 carbon atoms, or from about 12 to 18 carbon atoms. The tail group may contain aromatic moieties such as for example iso-octylphenyl. The surfactants can be nonionic, cationic, or anionic. Ionic surfactants such as cationic surfactants can be used to impart a charge to the sol-gel capsules alone or in combination with cationic polymers to produce highly charged sol-gel microcapsules. Other suitable surfactants are described in detail below.

One or more of the sunscreens used in a composition may be encapsulated; in some embodiments, all sunscreens used are encapsulated. Sunscreen actives may be encapsulated together, or may be encapsulated separately, in any combination, in the same or in different types of encapsulations. Generally, encapsulation involves trapping the sunscreen in, e.g., a vesicle. Depending on the vesicle of choice, the vesicle may break open when applied. Without being limited by theory, it is thought that the vesicle breaks open in various types of encapsulation due to friction, temperature, or pH from the skin or hair, or some combination of these. By choosing the appropriate capsule and additives for the system, the stability, durability, and/or SPF provided by the sunscreen additives and sunscreen/bodywashes of the invention can be increased.

Commercial embodiments of encapsulated sunscreens or vehicles suitable for encapsulating sunscreens include CATEZOMES (Engelhard Corp.), EUSOLEX UV PEARLS (EMD Biosciences), and others known in the art. Methods of encapsulation suitable for delivering benefit agents that are mixed with a bodywash composition are well-known in the art. See, e.g., U.S. Pat. Nos. 6,825,161; 6,436,375; 6,238,650; 6,468,509, 6,362,146; 6,074,630; 5,455,048; 5,770,556; 5,955,409; 5,876,755; 4,803,195; 5,508,259; 4,749,501; 6,248,703 5,476,660; and 4,904,524 and EP Pat. Nos. 0,254,447; 0,025,379; and 0,399,911.

One embodiment of a method of encapsulation of sunscreens is sol-gel encapsulation. This technique is described in, e.g., U.S. Pat. Nos. 6,238,650; 6,436,375, 6,303,149; and 6,468,509 and further herein. Any or all of the sunscreens and/or other active ingredients of the compositions of the invention may be encapsulated by such sol-gel encapsulation. The sol-gel capsules may be prepared so as to have a surface charge, e.g., a cationic charge. This is advantageous in that otherwise water-insoluble components may be encapsulated within the microcapsules, which are then freely miscible in water, e.g., without the need for an emulsifying agent. For example, in some embodiments, a UVA absorber, a UVB absorber (e.g., octyl methoxycinnamate) and/or a physical blocker, e.g., titanium dioxide, is provided as a silica sol-gel encapsulate, optionally with further ingredients including PVP, Chlorphenesin, and an antioxidant such as BHT. A commercial embodiment of such an encapsulation containing octyl methoxycinnamate, PVP, chlorphenesin, and BHT, is available under the trade name EUSOLEX UV PEARLS (EMD Biosciences). Such a silica sol-gel encapsulated UVB absorber, e.g., octyl methoxycinnamate, may be used in a sunscreen additive at a concentration that results in a final concentration of the UVB absorber of about 1% to about 40%, or about 2% to about 20%, or about 2% to about 10%, or about 5% to about 10%, or about 6%, 7%, 7.4%, 7.5%, 7.6%, 8%, or 9%. In some embodiments, the final concentration is about 7.6%. In other embodiments, more than one sunscreen is encapsulated as silica sol-gel encapsulate. In these embodiments, the final concentration of each of the sunscreens, independently, in the final sunscreen additive, is about 1% to about 40%, or about 2% to about 20%, or about 2% to about 10%, or about 1%, 2%, 3%, 4%, 5%, 6%, 7%, 7.5%, 8%, 9%, or 10%. The sunscreens may be encapsulated together or separately, or any combination thereof. In some embodiments, the invention provides an additive for addition to a bodywash that includes a sunscreen encapsulated in a sol-gel microcapsule and a cationic polymer (described below). Further ingredients in these embodiments may include a film former, antioxidant, preservative, chelating agent, thickener, emollient, and/or other active and inactive ingredients as described herein.

Other forms of immobilization or entrapment of sunscreen and other active components are also useful. For example, as a further variant of the use of chemical sunscreen agents, compositions of the invention may employ an organic sunscreen such as octyl methoxycinnamate trapped within a matrix. A commercial example of such a composition is found in SunCaps™) (trademark, SkinCeuticals) in which the organic sunscreen molecules are evenly distributed throughout the particle.

In some embodiments the invention provides microcapsules, e.g., sol-gel microcapsules (e.g., as described in U.S. Pat. Nos. 6,238,650; 6,436,375, 6,303,149; and 6,468,509) that act as a protective barrier on the skin when used either alone, or as an additive in a bodywash. In these embodiments, the sol-gel microcapsules may be used without any additional active ingredients (i.e., empty), providing a physical barrier, or they may be used with additional encapsulated active ingredients that enhance their barrier function. For example, the microcapsules may contain substances that act to screen toxic agents (e.g., biological or chemical warfare agents) or radiation (e.g., alpha, beta, or gamma radiation) partially or completely from penetrating the user's skin. In some embodiments, the microcapsules may contain one or more agents that absorb radiation, such as graphite, lead, tungsten, and others known in the art, or agents that reflect radiation such as ceramic beads. As the microcapsules may be designed so as to experience minimal or no breakage when applied to the skin, as well as to experience minimal penetration of the skin, it is possible to use even toxic substances (e.g., lead) that provide a screening effect, since these substances will not be released or will be released in only minimal amounts. The microcapsules are eventually removed from the skin through repeated washing and/or normal sloughing of the external skin cell layers. Especially for agents used for one-time or very few exposures, such as can occur for personnel engaged in combating or containing terrorist attacks or in warfare, the invention provides a means to deliver a last line of defense on the skin of personnel where the active used in the microcapsules may be one that is not appropriate for long-term use, but that is appropriate for a limited number of applications in order to protect the wearer from a greater risk (e.g., microcapsules encapsulating lead to protect against a radiation attack). Additives for protecting the user include agents that protect a user from the environment, including additives that protect fire fighters from the toxic agents generated in a fire, for protection of workers in factory environments that contain toxins, and for protection of individuals from harmful atmospheric compounds such as protection from acid rain.

In some embodiments, the active is encapsulated sol-gel microcapsules, such as silica sol-gel microcapsules. Such microcapsules are described in U.S. Pat. Nos. 6,238,650; 6,436,375, 6,303,149; and 6,468,509. Thus, in some embodiments the invention provides an additive for addition to a composition for topical application, where the additive comprises an encapsulated non-sunscreen active, and optionally further comprises a cationic polymer. In other embodiments the invention provides a composition for topical application that contains an additive, where the additive comprises an encapsulated non-sunscreen active, and optionally further comprises a cationic polymer. Further ingredients include film formers, antioxidants, preservatives, and other ingredients as listed herein. The composition for topical application may be, e.g., a bodywash.

Microcapsules of the present invention can have a positive charge density. The microcapsules of the present invention can have a positive charge. The positive charge can, for example, can impart improved emulsion stability and improve adhesion to the skin. While not being bound by theory, one framework commonly employed in the area of colloid sciences is the DLVO theory, which states that the stability of a particle in solution is dependent upon its total potential energy function, V_{T}. The theory recognizes that V_{T} is the balance of several competing contributions: the potential energy due to solvent, V_{S}, the potential energy due to attraction, V_{A}, and the potential energy due to repulsion, V_{R}. The potential energy due to repulsion, V_{R}, is an important contributor to the stability of the colloid. One aspect of V_{R} is the electrostatic repulsion, which is related to the square of the zeta potential. The zeta potential can be described in the following manner. Each particle has a liquid layer around it that can be viewed as existing as two parts; an inner region (Stern layer) where the ions are strongly bound and an outer (diffuse) region where they are less firmly associated. This system is referred to as the double layer. Within the diffuse layer there is a notional boundary inside which the ions and particles form a stable entity. When a particle moves, ions within the boundary move it. Those ions beyond the boundary stay with the bulk dispersant. The potential at this boundary (surface of hydrodynamic shear) is the zeta potential. Because the electrostatic repulsion of the repulsion potential, V_{R} is related to the square of the zeta potential, as the square of the zeta potential rises, the electrostatic repulsion rises, and the stability of the colloid rises. The positively charged microcapsules of the present invention thus exhibit stability in solution, while at the same time, potentially providing enhanced binding to the skin and hair.

Zeta potential can be calculated using theoretical models and an experimentally-determined electrophoretic mobility or dynamic electrophoretic mobility. Electrokinetic phenomena and electroacoustic phenomena are the usual sources of data for calculation of zeta potential. For example, electrophoresis is used for estimating zeta potential of particulates. Electrophoretic velocity is generally proportional to electrophoretic mobility, which is the measurable parameter. There are several theories that link electrophoretic mobility with zeta potential (see, for example, Lyklema, J. “Fundamentals of Interface and Colloid Science”, vol. 2, page. 3.208, 1995; and Hunter, R. J. “Foundations of Colloid Science”, Oxford University Press, 1989). Zeta potential can be determined, for example using microelectrophoresis or electrophoretic light scattering. With microelectrophoresis, images of the moving particles are used. In some cases, this method can be complicated by electro-osmosis at the walls of the sample cell.

Electrophoretic light scattering is based on dynamic light scattering. It allows measurement in an open cell, which eliminates the problem of electro-osmotic flow. Both these measuring techniques generally require dilution of the sample. Dilution is usually performed using equilibrium supernatant solution to minimize the effect of dilution on the zeta potential. In some cases, zeta potential can be measured electroacoustically. For example, the techniques of Colloid Vibration Current and Electric Sonic Amplitude can be used, (Dukhin, A. S, and Goetz, P. J. “Ultrasound for characterizing colloids”, Elsevier, 2002. reference). In some cases, the measurement of zeta potential provides a distribution of zeta potentials for the particles in the sample. In other cases, the methods provide a single zeta potential for the sample. Generally herein, where a reference to a zeta potential for a sample is described, it represents either the single measurement for the sample, or the mean, median or average of the distribution. In some cases the median value of the distribution of zeta potentials is used.

The zeta potential can be measured for instance on a Zetasizer instrument from Malvern Instruments, Malvern, UK, or on a ZetaPlus or ZetaPALS instrument from Brookhaven Instruments, Holtsville, N.Y.

In some embodiments, the microcapsules of the present invention have a zeta potential of at least about 5, 10, 12, 14, 16, 18, 20, 25, 30, 35, 40, 45, 50, 55, 60, 65, 70, 80, 90 or 100 mV. In some embodiments, the microcapsules of the present invention have a zeta potential of no more than about 15, 20, 25, 30, 35, 40, 45, 50, 55, 60, 65, 70, 80, 90, 100, 150, 200, 300, 400 or 500 mV. In some embodiments the zeta potential is between 10 and 70 mV, between 20 and 65 mV, between 25 and 65 mV, between 30 and 60 mV, between 30 and 100 mV, between 40 and 80 mV, between 70 and 100 mV or between 40 and 55 mV. In some embodiments, the microcapsules have a zeta potential of at least about 70 mV, in some embodiments, the microcapsules have a zeta potential of at least about 60 mV, in some embodiments, the microcapsules have a zeta potential of at least about 50 mV, in some embodiments, the microcapsules have a zeta potential of at least about 45 mV, in some embodiments, the microcapsules have a zeta potential of at least about 35 mV, in some embodiments, the microcapsules have a zeta potential of at least about 25 mV in some embodiments, the microcapsules have a zeta potential of at least about 15 mV.

The microcapsules of the present invention are usually dispersed in water or in an aqueous medium. The aqueous medium may contain salts, surfactants, viscosity modifiers, film formers, and other additives that may affect the zeta potential of the particles. It is known, for example that the zeta potential of a particle can be affected by the pH of the medium. The pH of the medium will have a particularly large effect on the zeta potential of a microcapsule when the microcapsule has ionizable, e.g. acidic or basic groups on its surface. For instance, where the microcapsule has a neutral acidic group, such as a carboxylic acid, that gives up a positively charged proton to the solution, the loss of the positively charged proton to the solution can give rise to one negative charge on the microcapsule surface. Conversely, a microcapsule surface with a neutral basic entity such as a trialkylamine, can become protonated in acidic solution, thus causing the microcapsule to take on a positive charge for each proton added. In both cases, the magnitude of the surface charge depends on the acidic or basic strengths of the surface groups and on the pH of the solution. In aqueous media, where the microcapsule has ionizable groups, the pH of the solution can have a dramatic affect on its zeta potential. For example, a microcapsule with ionizable carboxylic acid groups on the surface will have a negative zeta potential at high pH (basic conditions). If acid is added to this suspension then solution becomes more acidic, and the microcapsules tend to lose their negative charge. If enough acid is added to this suspension then a point will be reached where the charge will be neutralized. Where all of the charge is neutralized, there can be a point where microcapsules have zero zeta potential. This point is called the isoelectric point. The isoelectric point is normally the point where the colloidal system is least stable. Further addition of acid may cause a build up of positive charge on the microcapsules. Therefore a zeta potential versus pH curve will generally be positive at low pH and lower or negative at high pH

One aspect of the present invention is encapsulated actives wherein the capsules are positively charged at the pH at which the encapsulated additives are stored and applied. It will be understood by those skilled in the art that for topical applications, the compositions of the present invention will generally not be extremely acidic or extremely basic, because such solutions could be damaging to biological tissue such as skin and hair. Such solutions generally have a pH of 2-7. It will also be understood that where the application is not to a body, it may be desirable to have extremely high or low pH. For instance, in some cases, it will be useful to have an active agent with the capability of etching a surface such as a glass or a metal, where an very high or low pH is useful. Thus, the compositions of the present invention are formulated to have capsules of the desired zeta potential in the pH range of use.

The compounds of the present invention can also use buffered systems. Buffered systems use combinations of acidic and basic species in order to create a solution that has a pH which is less sensitive to the loss or addition of acidic or basic species. The buffered systems are used to stabilize the pH of the composition.

The capsules of the present invention will often have more than one acidic or basic group associated with the surface of the particle. For instance the particle may have a sol-gel coating, surfactants, and cationic components, each of which may have ionizable, acidic, or basic groups. The acidity of a group is can be represented by the pKa of the group. Under ideal conditions, the pKa is the pH at which the functional group is equally in its protonated and non-protonated forms. At a pH above the pKa most groups will be non-protonated. At a pH below the pKa, most of the groups will be protonated. Thus, where there are a variety of functional groups, each of these groups on the surface of the microcapsule that had a different pKa would give rise to a different zeta potential versus different pH response. The zeta potential on the capsule will be a composite of the zeta potentials that would be provided by each of these groups individually at any given pH. It would be understood by one skilled in the art to use compositions and processes in order to provide the relative amount of each of these groups to provide the desired zeta potential at the desired pH range of the composition.

The zeta potential can also be affected by the level of other salts in solution, also referred to as the ionic strength. In general, the higher the ionic strength, the more compressed is the double layer. The type of ion in solution can also affect the zeta potential. For example, multivalent ions will normally compress the double layer more than monovalent ions. As would be appreciated by one of skill in the art, the number and type of ion in the compositions of the present invention can be modified in order to produce the highly charged sol-gel microcapsules of the present invention.

One aspect of the invention is the use of non-ionizable cationic agents to create a positively charged microcapsule. For example, a quaternary ammonium functional group, such as that present in the polyquaterniums has nitrogen molecules which have 4 alkyl groups covalently attached. The positively charged nitrogen atoms have no protons to donate and no lone pairs are present to accept protons. This results in a positive charge on these molecules over a wide pH range. These groups are charged, but are thus considered neither acidic nor basic in the pH ranges useful in topical applications. Since the groups are neither acidic nor basic, they tend to provide microcapsules with a zeta potential which is less sensitive to changes in pH than for a microcapsule with a positively charged ionizable group. Having a zeta potential which is less sensitive to pH can be useful in providing freedom to formulate the compound containing the microcapsules, and for maintaining stability when the compound is exposed to conditions which might affect its pH.

While it is usually desired to have a high positive zeta potential on the microcapsules of the invention, there are cases, where it a negative zeta potential is desired. A negative zeta potential may be desired, for example for a wash off product, where there is less of a need for the capsules to adhere to skin and hair.

Methods of detecting the quantity of additives functionally remaining on the skin or hair are known in the art. One nonexclusive method is to measure the functionality of the additive on the skin or hair. This can be accomplished by applying an additive encapsulated in a microcapsule to the skin, and measuring the activity level of the additive. Another technique to measure the amount of additive functionally remaining on the skin is tape stripping, which is well known in the art. A microcapsule encapsulating an additive and dye compound is applied to the skin or hair. An adhesive material is applied to the skin and removed. The removed tape can then be analyzed. The level of the dye can be measured, which can then be correlated with the quantity of microcapsules bound to the skin. An electron microscope can also be used to detect whether the microcapsules are broken open, and to determine how many microcapsules are present per unit area. Multiple tape strippings can be performed sequentially. Each tape strip reveals a different level of the skin, so can be used to determine how deep the microcapsules penetrate.

In some embodiments wherein encapsulation, e.g., sol-gel microencapsulation, is utilized, the composition of the microcapsule, e.g., sol-gel microcapsule, may be varied so as to allow for varying amounts of the active within the microcapsule to be released. The microcapsules, e.g., sol-gel microcapsules, can be prepared so as to experience minimal or no breakage when applied to the skin and when left on the skin. Alternatively, the microcapsules, e.g., sol-gel microcapsules, can be prepared so as to experience various degrees of breakage, on average, when applied to the skin and when left on the skin. Thus, the microcapsules, e.g., sol-gel microcapsules, may be prepared so as to experience about 0% breakage, or breakage in a range from about 0.1, 0.5, 1, 2, 5, 10, 20, 30, 40, 50, 60, 70, 80, or 90% to about 0.5, 1, 2, 5, 10, 20, 30, 40, 50, 60, 70, 80, or 90%, after application (or application and rinsing in the case of a or bodywash containing the microcapsules). Furthermore, the microcapsules may be formulated so as to break open in response to conditions that occur on the skin, so that after application the microcapsules act to release their contents in a time-release or controlled manner. Non-limiting exemplary skin or hair conditions that can vary with the user's environment, the variation of which can trigger breakage of microcapsules, include pH, temperature, friction, exposure to light or air, pressure, enzymes, and the like.

In some cases the capsules are designed to break open and release their contents within a short period of time of contacting the skin or hair. For example, where a free-radical scavenger such as Vitamin E acetate is encapsulated, the capsules are formulated to break readily upon topical treatment, such that more than 50% of the capsules break open within 10, 15, 25, 35, or 50 minutes of topical application. In other cases, for example, where a skin lightener is encapsulated, the capsules can be formulated such that the active is released over a long period of time, for example, where 50% of the active is released after 4, 8, 12, 24, or 48 hours.

One way of controlling whether the microcapsules will tend to break is by controlling the conditions of manufacture including the temperature and the shear during mixing. In some cases, polymer wrapped or polymer coated microcapsules such as silica microcapsules will be able to stand higher salt concentrations and alkaline pH. The polymeric coatings are believed to assist in controlling breaking both by acting as a chemical barrier between the silica and the environment and also by providing more mechanical strength and elasticity.

The tendency of a microcapsule to break under shear can be measured by exposing the compound containing microcapsules to a set of conditions of shear (e.g. by controlling the RPM of stirring), temperature, and time, and analyzing the resulting mixture or aliquot of the mixture. The mixture can be analyzed, for example, by analyzing the solution in which the microcapsules are dispersed by high performance liquid chromatography (HPLC), which can be used to determine the amount of active or other component that has gone into the solution.

D. Cationic Component

One aspect of the invention is a composition with containing a cationic agent. In some embodiments the cationic agent is added to the sunscreen or non-sunscreen additive, imparting beneficial properties such as promoting attachment of the additive to skin or hair. In other embodiments the cationic agent is associated with the microcapsule, providing positive charge to the microcapsule. In some embodiments the additives, e.g., sunscreen additives and sunscreen/bodywashes of the invention include a cationic component. Without being bound by theory, it is thought that this component serves as a protein binder, to provide a positive charge to promote attachment of the composition to proteins of the skin and hair, thus increasing retention of the components, e.g., sunscreen, after rinse and during normal activities. This positive charge can create a strong affinity for the protein in the hair or skin. As described above, the cationic component can also create a positive charge on the surface of a microcapsule so as to stabilize the composition. Any means of imparting a positive charge to the microcapsule may be used.

In some embodiments any suitable cationic compound that may be useful to impart a positive charge on the microcapsule may be used.

In some embodiments, one or more cationic polymers are included in the composition. The term polymer means many “mers” or units. As used herein, the term polymer means a molecule having two or more repeating units. Various cationic polymers may be used. Examples of cationic polymers are described in U.S. Pat. Nos. 6,224,852; 3,816,616; 4,272,515; 4,298,494; 4,080,310; 4,048,301; 4,009,256; and 3,186,911. Cationic polymers are available commercially, e.g., from Union Carbide Corp. under the trademark POLYMER JR., from Celanese-Stein Hall under the trademark JAGUAR, from GAF Corporation under the tradename Gafquatm and from Merck & Co., Inc under the trademark MERQUAT by. Representative one are Merquat 100, a highly charged cationic dimethyldiallylammonium chloride homopolymer, and Merquat™ 550, a highly charged cationic copolymer prepared with dimethyldiallylammonium chloride and acrylamide. These materials are designated in the CTFA dictionary as Quaternium40 and Quaternium-41, respectively.

Suitable cationic polymers include Polyquaternium-4 (Celquat H-100; L200-supplier National Starch); Polyquaternium-7; Polyquaternium-10 (Celquat SC-240C; SC-230 M—supplier National Starch); (UCARE polymer series—JR-125, JR-400, LR-400, LR-30M, LK, supplier Amerchol); Polyquaternium-11 (Gafquat 734; 755N—supplier ISP); Polyquaternium-16 (Luviquat FC 370; FC550; FC905; HM-552 supplier by BASF); Polyquatemium-22, Polyquaternium-37, Polyquaternium-44, Polyquaternium-51, and Polyquaternium-64. PVP/Dimethylaminoethylmethacrylate (Copolymer 845; 937; 958—ISP supplier); Vinyl Caprolactam/PVP/Dimethylaminoethyl Methacrylate copolymer (Gaffix VC-713; H2OLD EP-1-supplier ISP); Chitosan (Kytamer L; Kytamer PC—supplier Amerchol); Polyquatemium-7 (Merquat 550-supplier Calgon); Polyquaternium-18 (Mirapol AZ-1 supplied by Rhone-Poulenc); Polyquaternium-24 (Quatrisoft Polymer LM-200-supplier Amerchol); Polyquaternium-28 (Gafquat HS-100-supplier ISP); Polyquaternium-46 (Luviquat Hold—supplier BASF); and Chitosan Glycolate (Hydagen CMF; CMFP—supplier Henkel); Hydroxyethyl Cetyldimonium Phosphate (Luviquat Mono CP—supplier BASF); and Guar Hydroxylpropyl Trimonium Chloride (Jaguar C series-13S, -14S, -17, 162, -2000, H1-CARE 1000-supplier Rhone-Poulenc).

Suitable cationic polymers also include Chitosan (Chitosan); Guar Hydroxypropyltrimonium Chloride (Guar Hydroxypropyltrimonium Chloride); Hydroxypropyl Guar Hydroxypropyltrimonium Chloride; Poly(Ethylenimine) (PEI-7 PEI-10 PEI-1500 . . . PEI-7500 PEI-14M); Poly(Methacrylamidopropyltrimonium Chloride/Methosulfate) (Polymethacrylamidopropyltrimonium Chloride); (Polyquaternium-2); Co(Hydroxyethylcellulose-g-Diallyldimethyl Ammonium Chloride) (Polyquaternium-4); Poly(Diallyldimethyl Ammonium Chloride) (Polyquaternium-6); Co(Diallyldimethyl Ammonium Chloride-Acrylamide) (Polyquaternium-7); Hydroxypropyltrimonium Hydroxyethylcellulose Chloride (Polyquatemium-10); Quaternized Co(Vinyl Pyrrolidone-Dimethylaminoethyl Methacrylate) (Polyquaternium-11); Co(Diallyldimethyl Ammonium Chloride-Acrylic Acid) (Polyquaternium-22); Hydroxypropyllauryldimonium Hydroxyethylcellulose Chloride (Polyquatemium-24); Co(Vinyl Pyrrolidone-Methacrylamidopropyl Trimethylammonium Chloride) (Polyquaternium-28); Co(Diallyldimethyl Ammonium Chloride-Acrylic Acid-Acrylamide) (Polyquaternium-39); Co(Vinyl Caprolactam-Vinyl Pyrrolidone-N-Vinyl-N-Methyl Imidazolinium Methosulfate) (Polyquaternium-46); Co(Vinyl Pyrrolidone-Dimethylaminopropyl Methacrylamide-Lauryl Dimethyl Methacrylamidopropyl Ammonium Chloride) (Polyquaternium-55); Co(Vinylpyrrolidone-Dimethylaminoethylmethacrylate)/Polycarbamyl Polyglycol Ester (PVP/Dimethylaminoethylmethacrylate/Polycarbamyl Polyglycol Ester); Co(Vinyl Pyrrolidone-Dimethylaminopropyl Methacrylamide) (PVP/DMAPA Copolymer); Co(Vinyl Pyrrolidone-Dimethylaminoethyl Methacrylate) (Vinyl Pyrrolidone/Dimethylaminoethylmethacrylate Copolymer); Co(Vinyl Pyrrolidone-Vinyl Caprolactam-Dimethylaminoethylmethacrylate) (Vinyl Pyrrolidone/Vinyl Caprolactam/Dimethylaminoethylmethacrylate Terpolymer); Co(Vinyl Pyrrolidone-Vinyl Caprolactam-Dimethylaminopropylmethacrylamide (Vinyl Pyrrolidone/Vinyl Caprolactam/Dimethylaminopropylmethacrylamide Terpolymer); Co(Vinyl Pyrrolidone-Vinyl imidazole) (Vinyl Pyrrolidone/Vinyl Imidazole Copolymer); and Co(Vinyl Pyrrolidone-3-methyl-1-Vinylimidazolinium methyl sulfate) (Vinyl Pyrrolidone/Vinylimidazolinium Methylsulfate Copolymer).

Some embodiments employ polyquaterniums. Quaternized material in powder form, not limited to the polyquaterniums, may also be used. Exemplary polyquaterniums of use in the invention include Polyquaternium-4, -7, -11, -22, -37, -44, -51, and -64. Without being limited by theory, it is believed that with the trapping of the encapsulate (e.g., sunscreen active inside the capsule) by the cationic component increases adhesion to the skin, making rinse off difficult and facilitating rendering the active substance, for instance, to the protein in the skin and hair. In other embodiments, other polyquaterniums may be useful for imparting a positive charge on the microcapsules.

Mixtures of the cationic components can be used. Uses of mixtures of cationic components can be made to increase solubility, improve processing, and to improve the properties of the compound, for example, enhancing adhesion to the skin and hair. Mixtures of different polyquaterniums can be used, for example, polyquaterniums with different molecular weight ranges, and mixtures of polyquaterniums and non-polyquaterniums can be used.

In some embodiments cationic surfactants can be used to impart a positive charge on the microcapsules. Cationic surfactants useful in the invention are described below. While the cationic component should be catioinic over all, the cationic component may also contain some anionic groups as well, and may be, for example amphoteric.

Useful in some embodiments of the invention is a dry cationic component, such as sold under the tradename CAE (Anjinomoto Co., Inc.), containing DL-pyrrolidone Carboxylic acid salt of L-Cocoyl Arginine Ethyl Ester, which is a cationic agent useful for binding to proteins and providing an antimicrobial effect.

In some embodiments, as an additive, the cationic component comprises about 0.1 to about 20%, or about 0.1 to about 10%, or about 0.5 to about 10%, or about 1 to about 10%, or about 0.5 to about 5%, or about 0.5 to about 3% or about 1 to about 5%, or about 1 to about 3%, or about 1% of the total composition. In some embodiments, the cationic component includes polyquatemium-4; in some embodiments the polyquaternium-4 is present at about 1%.

In some embodiments of active/bodywashes, e.g., sunscreen/bodywashes, the cationic component (e.g., cationic polymer) comprises about 0.03 to about 7%, or about 0.03 to about 4%, or about 0.2 to about 4%, or about 0.3 to about 4%, or about 0.2 to about 2%, or about 0.3 to about 4%, or about 0.3 to about 1%, or about 0.3 or 0.4% of the total composition. In some embodiments, the cationic component is polyquaternium-4; in some embodiments the polyquaternium-4 is present at about 0.33%.

In some embodiments, the cationic compound may be associated with the microcapsule in any suitable manner. In some embodiments the cationic compound is associated with the outside of the highly charged microcapsule. The cationic compound may be covalently bound to the microcapsule, may be bound non-covalently, or may exhibit a mixture of covalent and non-covalent binding. Non-limiting Examples of types non-covalent interactions between the cationic compound and the microcapsule are those due to electrostatic, hydrogen bonds, hydrophobic, or Van Der Waals forces.

In some embodiments it is desired to have an active contained within a microcapsule, while at the same time, providing another active outside the capsule, in the continuous phase of the composition. In one non-limiting example, it may be desired to have a moisturizer outside of the capsule for immediate access to the skin, while at the same time having a fragrance encapsulated within a capsule, for a longer, more controlled release of the fragrance. Another example of providing one active inside the capsule and another outside the capsule is in the area of tanning. In some cases a tanning active is used that is activated by another compound, for example by an amino acid. In such cases, the tanning active may be encapsulated within the microcapsule, while the activating compound is provided in the topical formulation, but outside of the microcapsules, and prevented from interacting with the tanning active on storage. Upon topical application, the sol-gel microcapsules are broken, for example, by friction, pressure, pH change, light, or enzymatic action, allowing release of the encapsulated active and allowing interaction of the activator with the tanning agent. This composition of one active encapsulated inside the microcapsule and one active outside the microcapsule allows for greater control of the tanning process and for improved storage life of the composition.

E. Film Formers

In some embodiments, compositions of the invention further include a component that provide a film barrier system, typically a hydrophobic layer that serves to maintain the residual sunscreen after rinse. Film barrier systems are well-known in the art and include, without limitation, petrolatum, silicon derivatives, and combinations thereof. Also useful are polymers with carboxylic ends which render themselves insoluble until neutralized. After being neutralized they can act as film formers. Film formers also include emollient esters, lanolin derivatives (e.g., acetylated lanolins), and superfatted oils. Film formers are available commercially, e.g., one exemplary film former is MOISTUREGUARD™, which contains petrolatum, dimethicone, stearamidopropyl dimethylamine stearate, and tocopheryl acetate, available from Engelhard.

It may also be desirable to add acrylic co-polymers to the formulations of the invention as film formers. An exemplary liquid acrylic copolymer formulation is DERMACRYL, marketed by National Starch and Chemical. Acrylic co-polymers may be included in sunscreen additives at about 0.1 to about 5%, or about 0.2 to about 3%, or about 0.2%, 0.3%, 0.4%, or 0.5%, or in sunscreen/bodywashes at about 0.05 to about 2%, or about 0.1 to about 1%, or about 0.05%, 0.1%, 0.2%, 0.3%, 0.4%, or 0.5%.

A secondary film former may also be used, e.g., keratin or other protein derivative in an amino acid complex such as cysteine.

The film former may be present in the sunscreen additive in the range of about 0.1 to about 25%, or about 1 to about 10%; or about 2 to about 6%; or about 3, 4, or 5%. In some embodiments, the film former MoistureGuard is used at a concentration of about 4.2%. Equivalent film formers, at equivalent concentrations, may also be used.

As noted, some preparations may perform more than one function, for example, inorganic blockers such as Tioveil and Spectraveil (both of the Tioxide Group), in certain variations, may be film-formers and may have advantageous uses here.

In addition, many emollients may also perform a film former function in that they provide a barrier on the skin. Thus, compositions of the invention may include water-insoluble emollients that include fatty acids such as oleic and stearic; fatty alcohols such as cetyl, and hexadecyl (ENJAY); esters such as diisopropyl adipate, benzoic acid esters of C₉-C₁₅ alcohols, and isononyl iso-nonanoate; alkanes such as mineral oil; silicones; such as dimethyl polysiloxane and ethers such as polyoxypropylene butyl ethers and polyoxypropylene cetyl ethers. If a water-insoluble emollient is used it may be in an amount from about 2% to about 15% by weight, or from about 4% to about 10%.

Other useful film formers include polythylenes, such as those available from New Phase Technologies as PERFORMALENE 400, a polyethylene having a molecular weight of 400. Another suitable water-proofing agent is polyethylene 2000 (molecular weight of 2000), which is available from New Phase Technologies as PERFORMALENE 2000.

Yet another suitable film former/waterproofing agent is synthetic wax, also available from New Phase Technologies as PERFORMA V-825. Still yet another suitable film former/waterproofing agent is octadecene/MA copolymer

Additional film formers which also may be used within the framework of the invention include any film former chemistry known in the art. Thus, suitable additional film formers include acacia gum, cellulose derivatives, guar derivatives and all those set forth on pages 68-69 of the C.T.F.A. Cosmetic Ingredient Handbook, First Edition, 1988, which is hereby incorporated by reference. Such film formers include acrylamides copolymer, acrylamide/sodium aciylate copolymer, acrylate/acrylamide copolymer, acrylate/ammonium methacrylate copolymer, acrylates copolymer, acrylates/diacetoneacrylamide copolymer, acrylic/acrylate copolymer, adipic acid/dimethylaminohydroxypropyl diethlenetnamine copolymer, adipic acid/epoxypropyl/diethlenetriamine copolymer, albumen, allyl stearate/VA copolymer, aminoethylacrylate phosphate/acrylate copolymer, ammonium acrylates copolymer, ammonium alginate, ammonium vinyl acetate/acrylates copolymer, AMP acrylates/diacetoneacrylamide copolymer, balsam canada, balsam oregon, balsam peru, balsam tolu, benzoi acid/phthalic anhydride/pentaerythritol/neopentyl glycol/palmitic acid copolymer, benzoin extract, butadiene/acrylonitrile copolymer, butylated urea-formaldehyde resin, butyl benzoic acid/phthalic anhydride trimethylolethane copolymer, butyl ester of ethylene maleic anhydride copolymer, butyl ester of PVM/MA copolymer, calcium carrageenean, calcium/sodium PVM/MA copolymer, carboxymethyl hydroxyethyl cellulose, cellulose gum, collodion, copal, corn starch/aciylainide/sodium acrylate copolymer, damar, diethylene glycolamine/epichlorohydrin/piperazine copolymer, DMJ-IF, dodecanedoic acid/cetearyl alcoholglycol copolymer, ethylcellulose, ethylene/acrylate copolymer, ethylene/maleic anhydride copolymer, ethylene/vinyl acetate copolymer, ethyl ester of PVM/fvIA copolymer, flexible collodian, gum benzoin, gutta percha, hydroxybutyl methylceflulose, hydroxyethylcellulose, hydroxyethyl ethyl cellulose, hydroxypropylcellulose, hydroxypropyl guar, hydroxypropyl methylcellulose, isopropyl ester of PVM/MA copolymer, maltodextrin, melamine/formaldehyde resin, methacryloyl ethyl betainelmethacrylates copolymer, nitrocellulose, octylacrylamide/acrylates/butylaminoethylmethaciylate copolymer, octylacrylamide/acrylates copolymer, phthalic anhydride/glycerin/gycidyl decanoate copolymer, phthalic/trimellitic/glycols copolymer, polyacrylamide, polyaciylamidomethylpropane sulfone acid, polyacrylic acid, polybutylene terephthalate, polychlorotrifluoroethylene, polyethylacrylate, polyethylene, polyethylene terephthalate, polyisobutene, Polyquaternium-1, Polyquaternium-2, Polyquaternium-4, Polyquaternium-5, Polyquatemium-6, Polyquaternium-7, Polyquaternium-8, Polyquaternium-9, Polyquaternium-10, Polyquaternium-11, Polyquaternium-12, Polyquaternium-13, Polyquatemium-14, Polyquaternium-15, polystyrene, polyvinyl acetate, polyvinyl alcohol, polyvinyl butyral, polyvinyl imidazolinium acetate, polyvinyl laurate, polyvinyl methyl ether, potassium carrageenan, PVM/MA copolymer, PVP, PVP/dimethylaminoethymethacrylate copolymer, PVP/eicosene copolymer, PVP/ethyl methacrylate/methacrylic acid copolymer, PVP/hexadecene copolymer, PVP/VA copolymer, PVP/vinyl acetate/itaconic acid copolymer, rosin, serum albumin, shellac, sodium acrylate/vinyl alcohol, copolymer, sodium carrageen, sodium polymethacrylate, sodium polystyrene sulfonate, starch/acrylates/acrylamide copolymer, starch diethylaminoethyl ether, steaxyvinyl ether/maleic anhydride copolymer, styrene/acrylate/acrylonitrile copolymer, styrene/acrylate/ammonium methacrylate copolymer, styrene/maleic anhydride copolymer, styrene/PVP copolymer, sucrose benzoate/sucrose acetate isobutyrate/butyl benzyl phthalate copolymer, sucrose benzoate/sucrose acetate isobutyrate/butyl benzyl phthalate/methyl methaciylate copolymer, sucrose benzoate/sucrose acetate isobutyrate copolymer, toluenesulfonamide/formaldehyde resin, tragacath gum, vinyl acetate/crotonates copolymer, vinyl acetate/crotonic acid copolymer, vinyl acetate/crotonic acid/methacryloxybenzophenon-1 copolymer, vinyl acetate/crotonic aid/vinyl neodecanoate copolymer, and zein

Additional film formers include those set forth in U.S. Pat. Nos. 6,838,419; 6,838,088; 6,780,422; 6,531,118; and 5,916,541, all of which are incorporated herein by reference

F. Other Components

A wide variety of additional components may be added to the compositions of the present invention, as long as the components are selected so as to avoid any undesirable reaction with the primary components (e.g., one or more of the sunscreen agents) of the composition. The CTFA Cosmetic Ingredient Handbook, Seventh Edition, 1997 and the Eighth Edition, 2000 (incorporated by reference herein), provide a broad source of possible cosmetic and pharmaceutical ingredients typically used in skin care compositions. Examples of such additional components include one or more of the following: Absorbents, abrasives, anticaking agents, antifoaming agents, binders, biological additives, buffering agents, bulking agents, chelating agents/sequestrants (e.g., disodium EDTA), chemical additives, colorants, cosmetic astringents, cosmetic biocides, denaturants, drug astringents, emollients (including glycerin alovera, and Vitamins A, C, and D [hydrating agents and skin protectants]), foam boosters, fragrance components, gums, humectants/moisturizers (including urea, guanidine, glycolic acid, polyhydroxy alcohols such as sorbitol, glycerin, hexanetriol, propylene glycol, hexylene glycol and the like, polyethylene glycol, sugars and starches, sugar and starch derivatives, D-panthenol, hyaluronic acid, lactamide monoethanolamine, acetamide monoethanolamine, and mixtures thereof), hydrotropes, neutralizing agents, opacifying agents and pigments, pH adjusters, plasticizers, preservatives, propellants, reducing agents, skin bleaching agents, skin protectants, solubilizing agents, and suspending agents (e.g., Carbomer 1382).

In some embodiments, anionic polymers are used. Suitable anionic polymers include Crosslinked, Hydrophobically Modified Poly(Acrylic Acid) (Acrylates/C10-30 Alkyl Acrylate Crosspolymer); Co(Alkyl Acrylate-Alkyl Methacrylate-Acrylic Acid-Methacrylic Acid) (Acrylates Copolymer); Co(Acrylic Acid-Methacrylic Acid-Alkyl Acrylates), Crosslinked (Acrylates Copolymer); Crosslinked, Co(Alkyl Acrylate-Methacrylic Acid) (Acrylates Copolymer); Co(Alkyl Acrylate-Methacrylic Acid-Acrylic Acid-Beheneth-25 Methacrylate) (Acrylates/Methacrylates/Beheneth-25 Methacrylate Copolymer); Co(Alkyl Acrylate-Methacrylic Acid-Steareth-20 Methacrylate) (Acrylates/Steareth-20 Methacrylate Copolymer); Co(Methacrylic Acid-Alkylene Succinic Acid-Alkyl Acrylate-Hydroxyalkyl Acrylate) Tetrapolymer (Acrylates/[C1-2 Succinates]/Hydroxyacrylates Copolymer); Alginic Acid (Alginic Acid or Algin for Sodium Alginate); Crosslinked Poly(Acrylic Acid) (Carbomer); Sodium Carboxymethylcellulose (Cellulose Gum); Co(Polyethylene Glycol-1,4-Cyclohexanedimethanol-Isophtalic Acid-Sulphonated Isophtalic Acid) (Diglycol/CHDM/Isophtalates/SIP Copolymer); Co(Methyl Vinyl Ether/Maleic Acid) (Methyl Vinyl Ether/Maleic Acid Copolymer); Co(Methyl Vinyl Ether/Maleic Acid-1,9-Decadiene) (PVM/MA Decadiene Crosspolymer); Monoalkyl Ester of Poly(Methyl Vinyl Ether/Maleic Acid) (Monoalkyl Ester of PVM/MA Copolymer); Co(Octylacrylamide-Acrylates-Butylaminoethyl Methacrylate) Terpolymer (Octylacrylamide-Acrylates-Butylaminoethyl Methacrylate Coolymer); Poly(Styrene Sulpfonate), Sodium Salt (Sodium Polystyrene Sulfonate); Co(Acrylic Acid-Methacrylic Acid-Alkyl Acrylates—Steareth-10 Allyl Ether) (Steareth-10 Allyl Ether/Acrylates Copolymer); Co(Vinyl Acetate-Crotonic Acid) (VA/Crotonates Copolymer); Co(Vinyl Acetate-Crotonic Acid-Vinyl Neodecanoate) Terpolymer (VA/Crotonates/Vinyl Neodecanoate Copolymer); and Xanthan Gum.

In some embodiments, the additives and bodywashes of the invention, e.g., sunscreen additives or sunscreen/bodywashes include a preservative. Exemplary preservatives useful in the invention include citric acid, tartaric acid, phosphoric acid, iminodiacetic acid, nitrilotriacetic acid, hydroxyethyleneaminodiacetic acid and ethylenediaminetetraacetic acid and salts thereof; para-hydroxybenzoates such as butyl paraben, methyl paraben and propyl paraben; imidazolines (e.g., imidiazolinylurea), triclosan, hydantoins (e.g., dimethyloldimethylhydantoin), isothiazolidinone compounds and mixtures thereof. Commercially available preservatives include KATHON CG and KATHON CGII, which contain methylchloroisothiazolinone and methylisothiazolinone (Rohm and Haas). When present, the quantity of preservative is in the range from 0.001 to 2%, or from 0.01 to 0.2%.

In certain embodiments the compositions of the invention include a chelating agent. Chelating agents are substances used to chelate or bind metallic ions, such as with a heterocyclic ring structure so that the ion is held by chemical bonds from each of the participating rings. Suitable chelating agents include ethylene diaminetetraacetic acid (EDTA), EDTA disodium, calcium disodium edetate, EDTA trisodium, EDTA tetrasodium and EDTA dipotassium. One or more chelating agents can optionally be included in the additives or additive/bodywashes in amounts ranging from about 0.001 to about 0.2 weight percent, or about 0.01% weight percent.

Thickening agents or gellants may be added as desired to adjust the texture and viscosity of the composition. Exemplary agents or gellants may be selected from Carbopol™ resins [e.g., 934, 971, 974, 980, 981] and Pemulen™ [TR-1 and TR-2][both Carbopol™ and Pemulen™ are registered trademarks of BF Goodrich], Noveon AA-1, ETD resins, and Ultrez™ resins [registered trademark, BF Goodrich]. In addition, carbomers might be useful for this purpose.

It may be desired to include a non-polar wax. Examples of such useful waxes include ester waxes, diester waxes, hydrocarbon waxes, silicone waxes and triglyceride waxes and mixtures thereof.

Other components may include a liquid hydrocarbon (similar to pentane), and/or a cationic foaming agent derived from arginine and or cysteine.

Further optional ingredients which can be present in the composition include fragrance, dyes, antimicrobial materials such as triclocarban, triclosan, iodophors, iodine formulations, phenolic compounds, e.g. hexachlorophene, and bisbiguanides, e.g. chlorhexidene gluconate, and the like. See, e.g., U.S. Pat. Nos. 6,827,795; 6,517,854; 6,010,817; 5,173,216; 5,719,113; 5,259,984; 5,562,912; 5,629,006; 5,728,662; 5,767,163; 5,750,579; 5,591,442; 5,650,143; 5,772,640; and 4,478,821.

The components of the composition are generally mixed in water.

G. Surfactants and Bodywashes

Compositions of the invention may be formulated as products for use as a wash-on formulation, for providing a cleaning function with respect to a surface. In some casese, the compositions are formulated for washing the skin, for example, bath or shower gels, hand washing compositions or facial washing liquids; pre- and post-shaving products; rinse-off, wipe-off and leave-on skin care products; products for washing the hair and for dental use. Shower gels are particularly exemplary product forms.

If it is desired to prepare a sunscreen/bodywash composition, the sunscreen additives of the invention may be combined with other ingredients to produce a bodywash (e.g., a liquid or solid formulation). The sunscreen/bodywash may include one or more surfactants. The use of surfactants in bodywashes is well-known in the art. Any surfactant known in the art and appropriate for a bodywash composition may be used. See, McCutcheon's Detergents & Emulsifiers, M.C. Publishing Co. (North American edition 1989); Schwartz, et al., Surface Active Agents, Their Chemistry and Technology, New York, Interscience Publishers, 1949, and U.S. Pat. Nos. 6,096,697; 4,741,855; 4,788,066; 5,104,646; 5,106,609; 2,658,072; 2,438,091; 2,528,378; 2,486,921; 2,486,922; 2,396,278; 2,979,465; 3,179,599; 5,322,643; 5,084,212; 3,332,880; 4,122,029; 4,265,878; 4,421,769; 3,929,678; 3,959,461; 4,387,090; 4,303,543; and 6,224,852; and in British Patent Nos. 848,224 and 791,415. Also see CTFA Cosmetic Ingredient Dictionary, 4^{th} Edition 1991, pages 509-514 for various long chain alkyl cationic surfactants; and Richmond, James M., Cationic Surfactants, Marcel Dekker, Inc., New York and Basel, 1990.

The surfactant(s) may be cationic, anionic, nonionic, zwitterionic, amphoteric, or any combination thereof.

Specific examples of anionic surfactants include those selected from the group consisting of alkyl and alkyl ether sulfates, sulfated monoglycerides, sulfonated olefins, alkyl aryl sulfonates, primary or secondary alkane sulfonates, alkyl sulfosuccinates, acyl taurates, acyl isethionates, alkyl glycerylether sulfonate, sulfonated methyl esters, sulfonated fatty acids, alkyl phosphates, ethoxylated alkyl phosphates, acyl glutamates, acyl sarcosinates, alkyl sulfoacetates, acylated peptides, alkyl ether carboxylates, acyl lactylates, anionic fluorosurfactants, and combinations thereof. Combinations of anionic surfactants can be used effectively in the present invention. Specific examples of alkyl sulfates that may be used are sodium, ammonium, potassium, magnesium, or TEA salts of lauryl or myristyl sulfate. Examples of alkyl ether sulfates that may be used include ammonium, sodium, magnesium, or TEA laureth-3 sulfate.

Another suitable class of anionic surfactants are the sulfated monoglycerides of the form R1CO—O—CH₂—C(OH)H—CH₂—O—SO₃M, wherein R1 is a saturated or unsaturated, branched or unbranched alkyl group from about 8 to about 24 carbon atoms, and M is a water-soluble cation such as ammonium, sodium, potassium, magnesium, triethanolamine, diethanolamine and monoethanolamine. An example of a sulfated monoglyceride is sodium cocomonoglyceride sulfate.

Other suitable anionic surfactants include olefin sulfonates of the form R1SO₃M, wherein R1 is a mono-olefin having from about 12 to about 24 carbon atoms, and M is a water-soluble cation such as ammonium, sodium, potassium, magnesium, triethanolamine, diethanolamine and monoethanolamine. An example of a sulfonated olefin is sodium C14/C16 alpha olefin sulfonate.

Other suitable anionic surfactants are the linear alkylbenzene sulfonates of the form R1-C₆H₄—SO₃M, wherein R1 is a saturated or unsaturated, branched or unbranched alkyl group from about 8 to about 24 carbon atoms, and M is a water-soluble cation such as ammonium, sodium, potassium, magnesium, triethanolamine, diethanolamine and monoethanolamine. An example of this anionic surfactant is sodium dodecylbenzene sulfonate.

Still other anionic surfactants suitable for the compositions of the present invention include the primary or secondary alkane sulfonates of the form R1 SO₃M, wherein R1 is a saturated or unsaturated, branched or unbranched alkyl chain from about 8 to about 24 carbon atoms, and M is a water-soluble cation such as ammonium, sodium, potassium, magnesium, triethanolamine, diethanolamine and monoethanolamine. An example of an alkane sulfonate useful herein is alkali metal or ammonium C13-C17 paraffin sulfonates.

Still other suitable anionic surfactants are the alkyl sulfosuccinates, which include disodium N-octadecylsulfosuccinamate; diammonium lauryl sulfosuccinate; tetrasodium N-(1,2-dicarboxyethyl)-N-octadecylsulfosuccinate; diamyl ester of sodium sulfosuccinic acid; dihexyl ester of sodium sulfosuccinic acid; and dioctyl esters of sodium sulfosuccinic acid.

Also useful are taurates that are based on taurine. Examples of taurates include N-alkyltaurines such as the one prepared by reacting dodecylamine with sodium isethionate as detailed in U.S. Pat. No. 2,658,072.

Another class of suitable anionic surfactants is the acyl isethionates. Nonlimiting examples of these acyl isethionates include ammonium cocoyl isethionate, sodium cocoyl isethionate, sodium lauroyl isethionate, and mixtures thereof.

Still other suitable anionic surfactants are the alkylglyceryl ether sulfonates of the form R1-OCH₂—C(OH)H—CH₂—SO₃M, wherein R1 is a saturated or unsaturated, branched or unbranched alkyl group from about 8 to about 24 carbon atoms, and M is a water-soluble cation such as ammonium, sodium, potassium, magnesium, triethanolamine, diethanolamine and monoethanolamine. One example is sodium cocoglyceryl ether sulfonate.

Other suitable anionic surfactants include: Sulfonated fatty acids of the form R1-CH(SO₄)—COOH and sulfonated methyl esters of the from R1-CH(SO₄)—CO—O—CH₃, where R1 is a saturated or unsaturated, branched or unbranched alkyl group from about 8 to about 24 carbon atoms (e.g., alpha sulphonated coconut fatty acid and lauryl methyl ester); phosphates such as monoalkyl, dialkyl, and trialkylphosphate salts formed by the reaction of phosphorous pentoxide with monohydric branched or unbranched alcohols having from about 8 to about 24 carbon atoms (e.g., sodium mono or dilaurylphosphate, ethoxylated monoalkyl phosphates, etc.); acyl glutamates corresponding to the formula R1CO—N(COOH)—CH₂CH₂—CO₂M wherein R1 is a saturated or unsaturated, branched or unbranched alkyl or alkenyl group of about 8 to about 24 carbon atoms, and M is a water-soluble cation (e.g., sodium lauroyl glutamate and sodium cocoyl glutamate); alkanoyl sarcosinates corresponding to the formula R1CON(CH₃)—CH₂CH₂—CO₂M wherein R1 is a saturated or unsaturated, branched or unbranched alkyl or alkenyl group of about 10 to about 20 carbon atoms, and M is a water-soluble cation (e.g., sodium lauroyl sarcosinate, sodium cocoyl sarcosinate, and ammonium lauroyl sarcosinate); alkyl ether carboxylates corresponding to the formula R1-(OCH₂CH₂)x—OCH₂—CO₂M wherein R1 is a saturated or unsaturated, branched or unbranched alkyl or alkenyl group of about 8 to about 24 carbon atoms, x is 1 to 10, and M is a water-soluble cation (e.g., sodium laureth carboxylate); acyl lactylates corresponding to the formula R1CO—[O—CH(CH₃)—CO]x—CO₂M wherein R1 is a saturated or unsaturated, branched or unbranched alkyl or alkenyl group of about 8 to about 24 carbon atoms, x is 3, and M is a water-soluble cation (e.g., sodium cocoyl lactylate); carboxylates, nonlimiting examples of which include sodium lauroyl carboxylate, sodium cocoyl carboxylate, and ammonium lauroyl carboxylate; anionic fluorosurfactants; and natural soaps derived from the saponification of vegetable and/or animal fats & oils examples of which include sodium laurate, sodium myristate, palmitate, stearate, tallowate, cocoate.

Any counter cation, M, can be used on the anionic surfactant. The counter cation may be, for example, selected from the group consisting of sodium, potassium, ammonium, monoethanolamine, diethanolamine, and triethanolamine.

Nonlimiting examples of nonionic surfactants that may be included in the compositions of the present invention include those selected from the group consisting of alkyl glucosides, alkyl polyglucosides, polyhydroxy fatty acid amides, alkoxylated fatty acid esters, sucrose esters, amine oxides, and mixtures thereof.

Alkyl glucosides and alkyl polyglucosides are useful herein, and can be broadly defined as condensation products of long chain alcohols, e.g., C₈₋₃₀ alcohols, with sugars or starches or sugar or starch polymers, i.e., glycosides or polyglycosides. These compounds can be represented by the formula (S)ₙ—O—R wherein S is a sugar moiety such as glucose, fructose, mannose, and galactose; n is an integer of from about 1 to about 1000, and R is a C₈₋₃₀ alkyl group. Examples of long chain alcohols from which the alkyl group can be derived include decyl alcohol, cetyl alcohol, stearyl alcohol, lauryl alcohol, myristyl alcohol, oleyl alcohol, and the like. Some examples of these surfactants include those wherein S is a glucose moiety, R is a C₈₋₂₀ alkyl group, and n is an integer of from about 1 to about 9. Commercially available examples of these surfactants include decyl polyglucoside (available as APG 325 CS from Henkel) and lauryl polyglucoside (available as APG 600CS and 625 CS from Henkel). Also useful are sucrose ester surfactants such as sucrose cocoate and sucrose laurate.

Other useful nonionic surfactants include polyhydroxy fatty acid amide surfactants, more specific examples of which include glucosamides Processes for making compositions containing polyhydroxy fatty acid amides are disclosed, for example, in G.B. Pat. Specification 809,060, published Feb. 18, 1959, by Thomas Hedley & Co., Ltd.; U.S. Pat. No. 2,965,576, to E. R. Wilson, issued Dec. 20, 1960; U.S. Pat. No. 2,703,798, to A. M. Schwartz, issued Mar. 8, 1955; and U.S. Pat. No. 1,985,424, to Piggott, issued Dec. 25, 1934.

Other examples of nonionic surfactants include amine oxides. Amine oxides correspond to the general formula R₁ R₂, R₃ N→O, wherein R₁ contains an alkyl, alkenyl or monohydroxy alkyl radical of from about 8 to about 18 carbon atoms, from 0 to about 10 ethylene oxide moieties, and from 0 to about 1 glyceryl moiety, and R₂ and R₃ contain from about 1 to about 3 carbon atoms and from 0 to about 1 hydroxy group, e.g., methyl, ethyl, propyl, hydroxyethyl, or hydroxypropyl radicals. The arrow in the formula is a conventional representation of a semipolar bond. Examples of amine oxides suitable for use in this invention include dimethyl-dodecylamine oxide, oleyldi(2-hydroxyethyl) amine oxide, dimethyloctylamine oxide, dimethyl-decylamine oxide, dimethyl-tetradecylamine oxide, 3,6,9-trioxaheptadecyldiethylamine oxide, di(2-hydroxyethyl)-tetradecylamine oxide, 2-dodecoxyethyldimethylamine oxide, 3-dodecoxy-2-hydroxypropyldi(3-hydroxypropyl)amine oxide, dimethylhexadecylamine oxide.

The term “amphoteric surfactant,” as used herein, is also intended to encompass zwitterionic surfactants, which are well known to formulators skilled in the art as a subset of amphoteric surfactants.

A wide variety of amphoteric lathering surfactants can be used in the compositions of the present invention. Particularly useful are those which are broadly described as derivatives of aliphatic secondary and tertiary amines, in some cases, the nitrogen is in a cationic state, in which the aliphatic radicals can be straight or branched chain and wherein one of the radicals contains an ionizable water solubilizing group, e.g., carboxy, sulfonate, sulfate, phosphate, or phosphonate.

Nonlimiting examples of amphoteric or zwitterionic surfactants are those selected from the group consisting of betaines, sultaines, hydroxysultaines, alkyliminoacetates, iminodialkanoates, aminoalkanoates, and mixtures thereof.

Examples of betaines include the higher alkyl betaines, such as coco dimethyl carboxymethyl betaine, lauryl dimethyl carboxymethyl betaine, lauryl dimethyl alphacarboxyethyl betaine, cetyl dimethyl carboxymethyl betaine, cetyl dimethyl betaine (available as Lonzaine 16SP from Lonza Corp.), lauryl bis-(2-hydroxyethyl)carboxymethyl betaine, oleyl dimethyl gamma-carboxypropyl betaine, lauryl bis-(2-hydroxypropyl)alpha-carboxyethyl betaine, coco dimethyl sulfopropyl betaine, lauryl dimethyl sulfoethyl betaine, lauryl bis-(2-hydroxyethyl)sulfopropyl betaine, amidobetaines and amidosulfobetaines (wherein the RCONH(CH.sub.2).sub.3 radical is attached to the nitrogen atom of the betaine), oleyl betaine (available as amphoteric Velvetex OLB-50 from Henkel), and cocamidopropyl betaine (available as Velvetex BK-35 and BA-35 from Henkel).

Examples of sultaines and hydroxysultaines include materials such as cocamidopropyl hydroxysultaine (available as Mirataine CBS from Rhone-Poulenc).

Examples of amphoteric surfactants of the present invention include the following compounds: Cetyl dimethyl betaine (this material also has the CTFA designation cetyl betaine); Cocamidopropylbetaine; Cocamidopropyl hydroxy sultaine. Examples of other useful amphoteric surfactants are alkyliminoacetates, and iminodialkanoates and aminoalkanoates of the formulas RN[(CH₂)CO₂M]₂ and RNH(CH₂).ₘ CO₂ M wherein m is from 1 to 4, R is a C₈-C₂₂ alkyl or alkenyl, and M is H, alkali metal, alkaline earth metal ammonium, or alkanolammonium. Also included are imidazolinium and ammonium derivatives. Specific examples of suitable amphoteric surfactants include sodium 3-dodecyl-aminopropionate, sodium 3-dodecylaminopropane sulfonate, N-higher alkyl aspartic acids such as those produced according to the teaching of U.S. Pat. No. 2,438,091; and the products sold under the trade name “Miranol” and described in U.S. Pat. No. 2,528,378. Other examples of useful amphoterics include amphoteric phosphates, such as coamidopropyl PG-dimonium chloride phosphate (commercially available as Monaquat PTC, from Mona Corp.). Also useful are amphoacetates such as disodium lauroamphodiacetate, sodium lauroamphoacetate, and mixtures thereof.

In some embodiments, the sunscreen/bodywashes of the invention include at least one cationic surfactant. As described above, cationic surfactants can be used to partially or fully provide a positive charge to the microcapsules of the invention. Many cationic surfactants are known to the art. Suitable cationic surfactants include, but are not limited to, fatty amines, di-fatty quaternary amines, tri-fatty quaternary amines, imidazolinium quaternary amines, and combinations thereof. Suitable fatty amines include monalkyl quaternary amines such as cetyltrimethylammonium bromide. A suitable quaternary amine is dialklamidoethyl hydroxyethylmonium methosulfate. By way of example, the following may be mentioned: stearyldimenthylbenzyl ammonium chloride; dodecyltrimethylammonium chloride; nonylbenzylethyldimethyl ammonium nitrate; tetradecylpyridinium bromide; laurylpyridinium chloride; cetylpyridinium chloride; laurylpyridinium chloride; laurylisoquinolium bromide; ditallow(Hydrogenated)dimethyl ammonium chloride; dilauryldimethyl ammonium chloride; and stearalkonium chloride.

Additional cationic surfactants are disclosed in U.S. Pat. No. 4,303,543 see column 4, lines 58 and column 5, lines 1-42, incorporated herein by references. Also see CTFA Cosmetic Ingredient Dictionary, 4th Edition 1991, pages 509-514 for various long chain alkyl cationic surfactants; incorporated herein by reference.

The total surfactants, e.g., cationic surfactant, may be present in the sunscreen/bodywash at about 0.1 to about 20%, or about 0.1 to about 10%, or about 0.1 to about 5%, or about 0.5 to about 5%, or about 1 to about 10%, or about 1 to about 5%, or about 0.1 to about 2%, or about 1 to about 2%. In some embodiments, a sunscreen/bodywash composition of the invention contains a surfactant, e.g., a cationic surfactant, at about 1%.

In addition to surfactants, other ingredients, as described above for additives, may be included in the additive/bodywash. Any component known in the art or useful in bodywashes may be used.

In some embodiments, soapless cleansers may be used in addition to, or instead of, soaps/surfactants. For example, Oilatum™ AD (registered trademark, Stiefel Laboratories), Aquanil™ (registered trademark, Person & Covey, Inc.), Cetaphil™ (trademark, Galderma Laboratories, Inc.) or SpectroDerm™ (registered trademark, Draxis Pharmaceutical Inc.), or their equivalents, may be utilized as a soapless component in the present invention.

As noted above, the sunscreen additives of the invention may also be combined with conventional bodywash compositions, as well as with shampoos for hair, and post-wash skincare compositions. Proportions for addition and mixing are given above as well as in more detail hereafter. An exemplary bodywash that may be used with additives of the invention is exemplified by SUAVE Body Wash. Ingredients of a typical SUAVE bodywash include: Water, Ammonium Lauryl Sulfate, Ammonium Laureth Sulfate, Cocamidopropyl Betaine, Fragrance, Glycerin, Hydrolyzed Milk Protein & Honey Extract, PEG-10 Sunflower Glycerides, Cocamide MEA, Guar Hydroxypropylrimonium Chloride, Acrylates Copolymer, PEG-5 Cocamide, Helianthus annuus (Sunflower) Seed Oil or Glycine Soja (Soybean) Oil, Tetrasoidum EDTA, Propylene Glycol, Ammonium Chloride, Sodium Hydroxide, Methylchloroisothiazolinone, Methylisothiazolinone, Titanium Dioxide (CI 77891)

### II. Methods

A. Preparation

The compositions of the invention may be prepared by any suitable method.

The encapsulated actives of the present invention can be made by chemical, physico-chemical, and physico-mechanical methods such as suspension, dispersion and emulsion, coacervation, layer-by-layer polymerization (L-B-L) assembly, sol-gel encapsulation, supercritical CO₂-assisted microencapsulation, spray-drying, multiple nozzle spraying, fluid-bed coating, polycondensation, centrifugal techniques, vacuum encapsulation, and electrostatic encapsulation.

Microencapsulation methods useful in the present invention is described, for example, in Ghosh, K., Functional Coatings and Microencapsulation: A General Perspective, Wiley-VCH, Weinheim, 2006, Benita, S., Microencapsulation: Methods and Industrial applications, Marcel Dekker, Inc., NY, 1996., Arshady, R., Microspheres, Microcapsules and Liposomes, Citrus Books, London, 1999, and Boissiere et al. J. Mater. Chem., 2006, 16, 1178.

The sol-gel microcapsules of the invention can be formed, for example, by using techniques described in U.S. Pat. Nos. 6,238,650; 6,436,375, 6,303,149; 6,468,509, and U.S. Patent Application No. 2005/0123611. In order to form highly charged microcapsules, a cationic agent may be incorporated into the microcapsule or become associated with the microcapsule. The cationic agent can, for example, be a cationic surfactant, a cationic polymer, or a both a cationic surfactant and a cationic polymer. The process for forming the microcapsules of the present invention generally involves mixing a gel precursor, an active ingredient, and a surfactant to form a mixture, emulsifying the mixture in an aqueous medium such that the gel precursor hydrolyzes to form a sol-gel ceramic microcapsule, resulting in at least a portion of the additive encapsulated within the microcapsule, and adding a cationic agent to impart a high zeta potential to the microcapsules. At least some of the cationic agent can be added prior to the formation of microcapsules. For instance, a cationic surfactant can be used in the initial formation stage in order to impart some charge. The cationic agent can also be incorporated after the formation of the microcapsules. For instance, a cationic polymer can be added to the solution containing the formed microcapsules containing the active ingredient. The cationic polymer, such as polyquaternium-4 can bind to the microcapsules, and/or become partially incorporated into the microcapsules, increasing the charge on the microcapsules.

One aspect of the invention comprises methods for preparation of highly charged sol-gel microcapsules comprising active ingredients. The methods include forming capsules using oil-in-water (O/W) emulsions, water-in-oil (W/O) emulsions, liposomes, micelles, and polymeric microspheres. The various methods allow for the encapsulation of any type of suitable ingredient, for example, those described herein. For example, an oil-in-water emulsion can be used for incorporating a non-polar active ingredient, where the non-polar active ingredient either comprises substantially all of the oil phase, or the non-polar active ingredient is mixed with other non-polar components, either active or inert. The non-polar components comprise the “oil” phase of the water-in-oil emulsion. The oil phase constitutes generally spheroidal liquid particles or droplets dispersed in the continuous aqueous phase. Hydrolysis of the gel precursor material produces a sol-el capsule which is formed around the non-polar components. The highly charged capsules are formed by incorporating a cationic agent into the capsules. In some embodiments, the cationic agent is added prior to formation of the sol-gel capsules. In some embodiments, the cationic agent is added during the formation of the sol-gel capsules. In some embodiments, the cationic agent is added after the formation of the sol-gel capsules.

One aspect of the invention comprises a method of manufacturing a highly charged sol-gel microcapsule comprising a non-polar active ingredient comprising: (a) combining the non-polar active ingredient, optional non-polar diluent, and aqueous phase; (b) agitating the combination formed in (a) to form an oil-in-water (O/W) emulsion wherein the non-polar active ingredient and optional non-polar diluent comprise the dispersed phase; (c) adding one or more surfactants; (d) adding a cationic agent; (e) adding a gel precursor to the O/W emulsion; and (f) mixing the composition from step (e) while the gel precursor hydrolyzes and sol-gel capsules are formed which comprise the non-polar active ingredient.

A water-in-oil emulsion provides for the encapsulation of polar and aqueous soluble active ingredients. In the water-in-oil method, the active ingredient or ingredients and optional polar diluent are dissolved or dispersed in an aqueous phase. A water-in-oil emulsion is formed, wherein the aqueous liquid particles or droplets are dispersed within a non-polar, aqueous immiscible “oil” phase. Hydrolysis of the gel precursor material produces a sol-gel capsule which is formed around the non-polar component. In some embodiments, the cationic agent is added prior to formation of the sol-gel capsules. In some embodiments, the cationic agent is added during the formation of the sol-gel capsules. In some embodiments, the cationic agent is added after the formation of the sol-gel capsules.

One aspect of the invention is a method of manufacturing a highly charged sol gel microcapsule comprising a polar active ingredient comprising: (a) combining the polar active ingredient, water, optional polar diluent, and a non-polar (oil) phase; (b) agitating the combination formed in (a) to form an water-in-oil (W/O) emulsion wherein the polar active ingredient, water, and optional polar diluent comprise the dispersed phase; (c) adding one or more surfactants; (d) adding a cationic agent; (e) adding a gel precursor to the W/O emulsion; and (f) mixing the composition from step (e) while the gel precursor hydrolyzes and sol-gel capsules are formed which comprise the polar active ingredient.

While we describe the invention with respect to the binary O/W or W/O, the methods of the invention can also be used in ternary, quaternary or higher emulsions such as W/O/W, O/W/O, W/O/W/O, etc.

The invention also provides for methods of forming highly charged sol-gel microcapsules using template within a solution, usually an aqueous solution. The template is generally structure dispersed within a continuous solution that comprises the active ingredient. The template is generally spheroidal, need not be a spheroid, and can have an elongated or irregular shape or distribution of shapes. The template can be a polymer microsphere, liposome, or micelle. Hydrolysis of the gel precursor material produces a sol-gel capsule which is formed around the template. The highly charged capsules are formed by incorporating a cationic agent into the capsules. In some embodiments, the cationic agent is added prior to formation of the sol-gel capsules. In some embodiments, the cationic agent is added during the formation of the sol-gel capsules. In some embodiments, the cationic agent is added after the formation of the sol-gel capsules.

One aspect of the invention is a method of forming a highly charged sol-gel microcapsule comprising an active ingredient within a template comprising: (a) forming a dispersion of templates, wherein the templates comprise an active ingredient, in an aqueous continuous phase; (b) adding a cationic agent; (c) adding a gel precursor to the aqueous continuous phase; and (d) mixing the composition from step (c) while the gel precursor hydrolyzes and sol-gel capsules are formed.

A non-polar active ingredient is generally an ingredient that is insoluble or sparingly soluble in water or in aqueous solution. The non-polar ingredient may be soluble in an oil such as mineral oil, palm oil, or silicone oil. It is understood in the art how to determine solubility in order to determine if a non-polar ingredient is suitable. In some embodiments, such as with the O/W method, the active ingredient or ingredients comprise the whole of the non-polar “oil” phase. In some embodiments of the O/W method, the non-polar active ingredients are dissolved or dispersed into an optional non-polar diluent. The non-polar diluent can be any suitable oil, wax, or solvent.

The non-polar phase can be dispersed within the aqueous phase by any suitable means. The dispersion of the non-polar phase in the aqueous phase is generally referred to as an emulsion. The formation of emulsions is known in the art. In some cases, a mixer, such as a mixer with a rotor-stator is used. Emulsions of the invention can also be formed using liquid gets, vibrating nozzles or other methods. The aqueous phase generally comprises at least 50% water. In some cases, the aqueous phase is substantially all water. In some cases, the aqueous phase comprises other co-solvents or other water soluble agents. Co-solvents, can be any water miscible solvent including, for example, methanol, ethanol, or ethylene glycol. The aqueous phase can also comprise other additives such as thickening agents, sugars, water soluble polymers, etc.

The oil-in-water emulsion or water-in-oil emulsion is generally stabilized using one or more surfactants. Suitable surfactants are described herein and known in the art.

In order to form the oil-in-water emulsion of the invention, surfactants with an HLB value above about 8 are generally used. In some cases, multiple surfactants are used. Where there are multiple surfactants, the combined HLB of the surfactants is generally used. The HLB of the surfactant or surfactants is between, for example, 7 and 13, 8 and 12, 9 and 11, 9.5 and 10.5. In some embodiments, the HLB of the surfactants is 8, 8.5, 9, 9.5, 10, 10.5, 11, 11.5 or 12.

In order to form the water-in-oil emulsion of the invention, surfactants with an HLB value below about 8 are generally used. In some cases, multiple surfactants are used. Where there are multiple surfactants, the combined HLB of the surfactants is generally used. The HLB of the surfactant or surfactants is between, for example, 2 and 7, 3 and 6, 4 and 5, or 3.5 and 4.5. In some embodiments, the HLB of the surfactants is 2, 2.5, 3, 3.5, 4, 4.5, 5, 5.5 or 6.

Suitable surfactants for forming the oil-in-water emulsion, water-in-oil emulsion, or template micelle include, for example, anionic, cationic, zwittenionic, semipolar, PEGylated, amine oxide and aminolipids. Suitable surfactants include: anionic—sodium oleate, sodium dodecyl sulfate, sodium diethylhexyl sulfosuccinate, sodium dimethylhexyl sulfosuccinate, sodium di-2-ethylacetate, sodium 2-ethylhexyl sulfate, sodium undecane-3-sulfate, sodium ethylphenylundecanoate, carboxylate soaps; cationic—dimethylammonium and trimethylammonium surfactants of chain length from 8 to 20 and with chloride, bromide or sulfate counterion, myristyl-gammapicolinium chloride and relatives with alkyl chain lengths from 8 to 18, benzalkonium benzoate, double-tailed quaternary ammonium surfactants with chain lengths between 8 and 18 carbons and bromide, chloride or sulfate counterions; nonionic: PEGylated surfactants of the form CₙEₘ where the alkane chain length n is from 6 to 20 carbons and the average number of ethylene oxide groups m is from 2 to 80, ethoxylated cholesterol; zwitterionics and semipolars—N,N,N-trimethylaminodecanoimide, amine oxide surfactants with alkyl chain length from 8 to 18 carbons; dodecyldimethylammoniopropane-1-sulfate, dodecyldimethylammoniobutyrate, dodecyltrimethylene di(ammonium chloride); decylmethylsulfonediimine; dimethyleicosylammoniohexanoate and relatives of these zwitterionics and semipolars with alkyl chain lengths from 8 to 20.

The cationic agent or cationic component used in the method to impart the high charge can be any suitable cationic agent described herein or known in the art including a cationic surfactant, a cationic polymer, or a both a cationic surfactant and a cationic polymer. The cationic polymer can comprise a polyquaternium, such as polyquatemium-4, -7, -11, -22, -27, -44, 51, or -64. In one exemplary embodiment, the cationic polymer is polyquaternium-4. In some embodiments, the cationic agent can also comprise a proton donor or lewis acid.

The point in the process where the cationic agent is introduced into the reaction mixture can be important with respect to the production of highly charged sol-gel capsules. The point of addition will depend, for example, on the type of reaction conditions and the type of cationic agent or agents employed. In some embodiments, the cationic agent is added prior to the hydrolysis of the gel precursor. In these cases, the cationic agent will often be added just before, during, or just after the addition of the gel precursor.

In some cases, the cationic agent is added during the hydrolysis of the gel precursor and formation of the sol-gel capsule. While not being bound by theory, it is believed that the presence of the cationic agent or addition of the cationic agent during formation of the capsule can result in incorporation of the cationic agent into the wall of the capsule. It is believed that in some cases, this type of addition can result in improved stability of the cationic charge.

In some cases, the cationic agent is added subsequent to the formation of the capsule, thus providing a coating of the cationic agent onto the outside of the capsule. While not being bound by theory, it is believed that treatment of the capsules with the cationic agent subsequent to the formation of the sol-gel capsule can result in the cationic agent being concentrated on the outermost portion of the sol-gel capsule, which can provide a high amount of charge for a given amount of cationic agent.

The cationic agent can be added at more than one point in the process. In some cases, more than one cationic agent is used, each of which is added at a different point in the process. For example, in one embodiment a first cationic agent comprising, for example, a cationic surfactant is added before addition of the gel precursor, and during or subsequent to formation of the sol-gel capsules a second cationic agent, for example, a polymeric cationic agent such as a polyquaternium is added. In this manner the combination of cationic agents can act together to create the highly charged sol-gel capsules of the invention.

The gel precursor can be any suitable sol-gel forming material described herein or known in the art. The gel precursor can be, for example, a silica-based gel precursors include tetramethoxysilane (TMOS), tetraethoxysilane (TEOS), tetrabutoxysilane (TBOS), tetrapropoxysilane (TPOS), polydiethoxysilane, methyltrimethoxysilane, methyltriethoxysilane, ethyltriethoxysilane, octylpolysilsesquioxane and hexylpolysilsesquioxane. The gel precursor is added to the oil-in-water emulsion, and the pH is adjusted in order to cause the gel-precursor to hydrolyze and form the sol-gel capsule. The reaction is carried out with mixing at a rate such that the sol-gel reaction occurs at the interface between the oil and water, creating the sol-gel capsule. In some embodiments the pH is raised (made basic) in order to form the sol-gel capsule. In some embodiments, the pH is lowered (made acidic) in order to form the sol-gel capsule. In some embodiments, the pH is lowered to between 2 and 6, 3 and 5, 3 and 4, or 3.2 and 3.8. In some embodiments the pH is lowered to 2, 2.5, 3, 3.2, 3.3, 3.4, 3.5, 3.6, 3.7, 3.8, 3.9, 4.0, 4.2, 4.4, 4.6, 4.8, 5, 5.5, or 6. The hydrolysis of the gel precursor generally requires the presence of water. In the case of the oil-in-water emulsion, the water for hydrolysis can be provided from the continuous aqueous phase of the emulsion. In the case of the water-in-oil emulsion, the water can be provided as part of the polar dispersed phase, and/or water can be added to the reaction mixture after formation of the emulsion in order to facilitate hydrolysis.

The size of the sol gel capsules formed is determined, at least in part, by the conditions of the reaction including the size of the original emulsion, and the conditions used for formation of the sol-gel capsules. A distribution of capsule sizes is generally obtained. The sol-gel capsules can also be fractionated into a desired size range after capsule formation. Fractionation can be carried out by methods known in the art such as selective precipitation, or by using filters or sieves in order to pass a selected size range and retain the rest. The size of the sol-gel capsules can be modified in order to suit a particular application. In some embodiments, the mean, median, or average size of the capsules is between 10 nm and 1 mm, between 10 nm and 1 μm, between 1 μm and 100 μm, 10 μm and 50 μm, 50 μm and 200 μm, or between 200 μm and 500 μm. In some embodiments, the mean, median, or average size of the capsules is between 1 nm and 10 nm, 10 nm and 100 nm, 100 nm and 1 μm, 1 μm and 10 μm, 10 μm and 100 μm, 100 μm and 1 mm, 1 mm-10 mm, or larger. In some embodiments, the mean, median, or average size of the capsules is within plus or minus 10% of 1 nm, 10 nm, 25 nm, 50 nm, 75 nm, 90 mm, 100 nm, 250 nm, 500 nm, 750 nm, 900 nm, 1 μm, 10 μm, 25 μm, 50 μm, 75 μm, 90 μm, 100 μm, 250 μm, 500 μm, 750 μm, 900 μm, 1 mm or larger.

The sol gel capsules can be isolated from the reaction mixture, for example by filtration or precipitation. In addition to isolation of the capsules from the solution, these processes can affect the size distribution of the sol-gel capsules. The capsules can be filtered using standard filtration equipment. In some cases a vacuum or pressure is used to facilitate the filtration process. The capsules can then be rinsed to remove impurities from the reaction mixture including residual ethanol and/or unreacted gel precursor. The capsules can be rinsed with any suitable solvent. In some embodiments, the capsules are rinsed with water. The rinsing steps can also be used to add other components to the capsules. For example, a rinse using a solvent comprising a cationic component can result in increasing the charge on the microcapsules.

The sol-gel capsules of the present invention can be dried. In some cases, the dried sol-gel capsules have better shelf life stability than the wet capsules. In some cases, the dried capsules are more suitable for incorporation into a formulation, for example a non-polar formulation for products such as wash-on or leave-on products. Drying can be accomplished by any suitable means including passive exposure to heat and dry air or with spray-dry machinery. In some cases the capsules are dried at room temperature, in some cases the capsules are dried at between room temperature and 50° C.

The methods of the invention can produce highly charged microcapsules. One method for measuring the charge on the microcapsule is with zeta potential. The methods produce capsules having a zeta potential of at least 5, 10, 12, 14, 16, 18, 20, 25, 30, 35, 40, 45, 50, 55, 60, 65, 70, 80, 90 or 100 mV. In some embodiments, the microcapsules of the present invention have a zeta potential of no more than 15, 20, 25, 30, 35, 40, 45, 50, 55, 60, 65, 70, 80, 90, 100, 150, 200, 300, 400 or 500 mV. In some embodiments the zeta potential is between 10 and 70 mV, between 20 and 65 mV, between 25 and 65 mV, between 30 and 60 mV, between 30 and 100 mV, between 40 and 80 mV, between 70 and 100 mV or between 40 and 55 mV. In some embodiments, the microcapsules have a zeta potential of at least 70 mV, in some embodiments, the microcapsules have a zeta potential of at least 65 mV, in some embodiments, the microcapsules have a zeta potential of at least 60 mV, in some embodiments, the microcapsules have a zeta potential of at least 55 mV, in some embodiments, the microcapsules have a zeta potential of at least 50 mV, in some embodiments, the microcapsules have a zeta potential of at least 45 mV, in some embodiments, the microcapsules have a zeta potential of at least 35 mV, in some embodiments, the microcapsules have a zeta potential of at least 25 mV in some embodiments, the microcapsules have a zeta potential of at least 15 mV.

In one aspect of the invention, the methods of the invention produce capsules with a zeta potential that is higher than the zeta potential without the cationic agent. In some embodments, the zeta potential of the capsule is 5%, 10%, 20%, 30%, 40%, 50%, 60%, 70%, 80%, 90%, or 1 times, 2 times, 3 times, 4 times, 5 times, 10 times, 20 times, 50 times, 100 times or more than the zeta potential of the capsule without the cationic agent. In some embodiments the zeta potential of the capsule is 5% to 10%, 10% to 20%, 20% to 50%, 50% to 90%, 1 to 2 times, 2 to 5 times, 5 to 10 times, 10 to 100 times or more than the zeta potential of the capsule without the cationic agent.

For the methods of the invention, in some cases, the steps are carried out in the order that they are listed. In some cases, where appropriate, the order of the steps can be different than the order listed

In the methods which utilize a template for the formation of a highly charged sol-gel microcapsule, the template is generally a microsphere, liposome or micelle. Where the template is a microsphere, it is generally a polymeric microsphere.

Polymeric microspheres of the present invention are generally microspheres formed (at least in part) from a crosslinkable polymer. The highly charged sol-gel microspheres may be employed, for example, as drug delivery agents, tissue bulking agents, tissue engineering agents, and embolization agents. There are numerous methods known for preparing polymeric microspheres. These methods include dispersion polymerization of the monomer, potentiometric dispersion of a dissolved crosslinkable polymer within an emulsifying solution followed by solvent evaporation, electrostatically controlled extrusion, and injection of a dissolved crosslinkable polymer into an emulsifying solution through a porous membrane followed by solvent evaporation.

In some cases, the polymeric microsphere template is porous. Suitable porous template polymers include, for example, alginates, polysaccharides, carrageenans, chitosan, hyaluronic acid, or other ionically crosslinkable polymers (also known as “shape-forming agents”), such as the classes of carboxylic-, sulfate-, or amine-functionalized polymers. The template polymer can also be generated from a blend of one or more of the above synthetic or naturally occurring materials, or derivatives thereof. In one preferred embodiment of the invention, the template polymer is an alginate, which is ionically crosslinkable. Polymeric microspheres can also be made from wide variety of generally chemically crosslinkable polymers such as, for example, vinyl polymers, polyacrylamides, polyethylene glycol, polyamides, polyureas, polyurethranes, polyvinyl alcohols, and derivatives thereof. For some (e.g., embolic) applications, a hydrophilic polymer, such as polyvinyl alcohol, will be preferred.

Other polymers suitable for the production of polymeric microspheres are ethylene/acrylic acid copolymer, HDI/trimethylol hexyllactone copolymer and silica, polymethyl methacrylate, methyl methacrylate copolymer, nylon 6, nylon 12, polyethylene, polymethylsilsesquioxane, and polystyrene

Suitable microsphere to serve as templates for the present invention include microspheres commercially available from Kobo Products, Inc. including EA-209, BPD-500W, BPD-500, BPD-500T, BPA-500, MP-2200, SunPMMA-S, BPA-500X, MSP-825, MSP-930, SunPMMA-P, TR-1, POMP610, SP-500, SP-10, SP-10L, CL-1080, CL-2080, TOSPEARL® 120A, TOSPEARL® 145A, TOSPEARL® 2000B, TOSPEARL® 3000A, TOSPEARL® 150K, TOSPEARL® 1110A.

In some cases, microspheres having an active ingredient that can be used as templates for forming highly charged sol-gel capsules are commercially available, such as those from Salvona L.L.C., New Jersey including: 7010 HydroSal™ Lift, 7014 HydroSal™ NanoFresh, 7015 HydroSal™ Neutralizer, 7020-SS HydroSal™ Sal Silk, 202 Sebum Control, 2002 MultiSal™ Flavor/Cooling (Lip Care), 2101 MultiSal™ Vitamin C+E, 2104 MultiSal™ SalCool™, 2105 MultiSal™ Salicylic Acid 10, 2106 MultiSal™ Salicylic Acid 30, 2106-BW MultiSal™ Salicylic Acid 20, 2107 MultiSal™ AI (Anti-Inflammatory), 2110 MultiSal™ LipVantage, 2111 MultiSal™ Silicone, 2115 MultiSal™ Collagen Tripeptide, 2401 MultiSal™ Fragrance, 2403 MultiSal™ Menthol, 2801 MultiSal™ Flavor/Cooling (Oral Care), 105 SalSphere™ Moisture Key, 4201 SalSphere™ Anti Frizz, 4221-1, SalSphere™ Vita Hair, 4222 SalSphere™ Color Guard, 4308 SalSphere™ Resveratrol.

The template for forming the highly charged microcapsule of the present invention can be a liposome. A liposome is generally a substantially spherical vesicle or capsule generally comprised of amphipathic molecules (e.g., having both a hydrophobic (nonpolar) portion and a hydrophilic (polar) portion). Typically, the liposome can be produced as a single (unilamellar) closed bilayer or a multicompartment (multilamellar) closed bilayer. The liposome can be formed by natural lipids, synthetic lipids, or a combination thereof. In some embodiments, the liposome comprises one or more phospholipids. In a some embodiments, the liposome comprises one or more additives, for example, a membrane stabilizer, an isotonic agent (e.g., sugars, sodium chloride, polyalcohols such as mannitol, sorbitol, and the like, a pH adjusting agent (e.g., a base, a basic amino acid, an acidic amino acid, sodium phosphate, sodium carbonate, and the like, present in an amount to adjust the liposome to a desired pH), an aggregation minimizer (e.g., a surfactant (e.g., polysorbates, poloxamers), polysaccharide, liposomal surface carboxyl groups, and the like), or a combination thereof. Lipids for use in the present invention include, but are not limited to, lecithin (soy or egg; phosphatidylcholine), dipalmitoylphosphatidylcholine, dimyristoylphosphatidylcholine, distearoylphosphatidylcholine, dicetylphosphate, phosphatidylglycerol, hydrogenated phosphatidylcholine, phosphatidic acid, a phospholipid, cholesterol, phosphatidylinositol, a glycolipid, phosphatidylethanolamine, phosphatidylserine, a maleimidyl-derivatized phospholipid (e.g., N-[4(p-malei-midophenyl)butyryl]phosphatidylethanolamine), dioleylphosphatidylcholine, dipalmitoylphosphatidylglycerol, dimyristoylphosphatidic acid, or a combination thereof. The liposome generally comprises a polar (aqueous) interior which can be used for the formation of highly charged sol-gel microspheres comprising polar active ingredients.

In one method, a “Phase I,” which is a “water phase,” is prepared by mixing the more water-soluble components of the composition. For example, Polyquaternium-4, a film former (e.g., in MOISTUREGUARD), and encapsulated sunscreen (e.g., in UV PEARLS), may be mixed until uniform. A “Phase II,” which is an “oil phase,” is prepared by mixing the more hydrophobic components of the composition. For example, Avobenzone (e.g., PARSOL 1789) may be mixed with Octocrylene, with heating, until dissolved. Then Phase I and Phase II are combined with gentle agitation, until a uniform composition is obtained (Phase III). Phase III may be further combined with a bodywash composition (e.g., SUAVE bodywash) and mixed until uniform. A further sunscreen, such as titanium dioxide, may be added to the Phase III/bodywash composition and mixed until uniform. Alternatively, the sunscreen may be added before addition to the bodywash or soap to provide an additive ready for formulation with a bodywash or soap.

In some embodiments of the invention, the highly charged microcapsules of the invention may be prepared by mixing the microcapsule with a cationic compound to impart the high positive charge density onto the microcapsule. In some embodiments, the cationic compound added to the microcapsule is a cationic polymer. The cationic polymer may be, for example, a polyquarternium. The polyquaternium may be, for example, polyquaternium-4.

In one embodiment, the cationic compound is associated with the outside of the highly charged microcapsule. In a further embodiment, the cationic compound is covalently bound to the microcapsule. In another embodiment, the cationic compound is noncovalently bound to the microcapsule. The interaction between the cationic compound and the microcapsule may be, for example, an electrostatic, ionic, or a Van Der Waals attraction.

B. Use

The highly charged sol-gel capsules containing active agents are useful in many applications. The highly charged microcapsules are used for example for agricultural, textile, industrial, transportation, marine, pharmaceutical, or personal care applications. The sol-gel capsules of the invention can be used as wash-on or as leave-on formulations.

Additives, e.g., sunscreen additives of the invention which are incorporated into highly charged sol-gel capsulse can be designed to be used in combination with a bodywash. Thus, the compositions of the invention can be designed to be applied while washing. This characteristic facilitates ease of use and may have the added benefit of being cumulative. Compositions of the present invention are readily applied during washing in a suitable or effective amount and may be generally applied all over the body. Shampoos may be applied specifically to the hair. A selected amount of a composition may be applied directly to the skin or may be used through intermediate application to a washcloth, pad, sponge, or other applicator. After lathering, dirt and sloughed-off skin may be washed away by rinsing with water leaving behind one or more of the additives, e.g., sunscreen components. Additives of the invention, e.g., sunscreen additives of the invention are also useful in hair shampoos and conditioners, and in after-wash lotions.

Thus, methods of the invention include methods for protection of skin from sunlight, comprising applying a bodywash comprising a sunscreen to the skin, wherein after application of the bodywash to skin and rinsing, the skin is protected from sunlight with an average SPF of at least about 2, 3, 4, 5, 6, 7, 8, 9, 10, 11, 12, 13, 14, 15, 16, 17, 18, 19, 20, or more than 20. In some embodiments, the skin is protected from sunlight with an average SPF of at least about 2. In some embodiments, the skin is protected from sunlight with an average SPF of at least about 5. In some embodiments, the skin is protected from sunlight with an average SPF of at least about 10. In some embodiments, the skin is protected from sunlight with an average SPF of at least about 15. In some embodiments, the bodywash is applied more than once; in these cases, the SPF may be cumulative and can increase with the second wash to, e.g., an average of more than 2, 5, 6, 7, 8, 9, 10, 11, 12, 13, 15, 15, 16, 17, 18, 19, 20, 21, 22, 23, 24, 25, 30, 35, 40, 45, or more than about 45. In some embodiments the bodywash is applied once per day. In some embodiments, the bodywash is applied more than once per day, for example, 2, 3, 4, or more than 4 times per day. In some embodiments, the bodywash is applied about every other day. In some embodiments, the body wash is applied about 10, 8, 7, 6, 5, 4, 3, 2 or 1 time per week.

In these methods, the active additive, e.g., sunscreen, often does not penetrate beyond a certain level in the skin, typically due to encapsulation. Thus, in some embodiments of the methods of the invention, the active additive, e.g., sunscreen, does not penetrate more than about 10, 20, 25, 30, 35, 40, 45, or 50 microns into the skin with one washing with a bodywash containing the additive. In some embodiments, the active additive, e.g., sunscreen, does not penetrate more than about 10, 20, 25, 30, 35, 40, 45, 50, 60, 70, 80, 90, 100, 120, or 150 microns into the skin, even with repeated washings.

In other embodiments the additive is designed to penetrate into the skin, thus, in these embodiments, the active additive penetrates to at least about 10, 20, 25, 30, 35, 40, 45, or 50 microns into the skin with one washing with a bodywash containing the additive. In some embodiments, the active additive penetrates more than about 10, 20, 25, 30, 35, 40, 45, 50, 60, 70, 80, 90, 100, 120, or 150 microns into the skin. In some embodiments this penetration occurs with a single washing and rinsing. In some embodiments this penetration occurs with repeated washings and rinsings.

Methods of the invention also include methods for protection of skin from sunlight, comprising applying a leave-on formulation comprising applying a sunscreen encapsulated in a highly charged sol-gel capsule to the skin, wherein after application of the leave-on formulation, the skin is protected from sunlight with an average SPF of at least about 2, 3, 4, 5, 6, 7, 8, 9, 10, 11, 12, 13, 14, 15, 16, 17, 18, 19, 20, or more than 20. In some embodiments, the skin is protected from sunlight with an average SPF of at least about 2. In some embodiments, the skin is protected from sunlight with an average SPF of at least about 5. In some embodiments, the skin is protected from sunlight with an average SPF of at least about 10. In some embodiments, the skin is protected from sunlight with an average SPF of at least about 15. In some embodiments, the leave-on formulation is applied more than once; in these cases, the SPF may be cumulative and can increase with the second wash to, e.g., an average of more than 2, 5, 6, 7, 8, 9, 10, 11, 12, 13, 15, 15, 16, 17, 18, 19, 20, 21, 22, 23, 24, 25, 30, 35, 40, 45, or more than about 45. In some embodiments the leave-on formulation is applied once per day. In some embodiments, the leave-on formulation is applied more than once per day, for example, 2, 3, 4, or more than 4 times per day. In some embodiments, the leave-on formulation is applied about every other day. In some embodiments, the body wash is applied about 10, 8, 7, 6, 5, 4, 3, 2 or 1 time per week.

In these methods, the active additive, e.g., sunscreen, often does not penetrate beyond a certain level in the skin, typically due to encapsulation. Thus, in some embodiments of the methods of the invention, the active additive, e.g., sunscreen, does not penetrate more than about 10, 20, 25, 30, 35, 40, 45, or 50 microns into the skin with one application with a leave-on formulation containing the additive. In some embodiments, the active additive, e.g., sunscreen, does not penetrate more than about 10, 20, 25, 30, 35, 40, 45, 50, 60, 70, 80, 90, 100, 120, or 150 microns into the skin, even with repeated applications.

In other embodiments the additive, is designed to penetrate into the skin, thus, in these embodiments, the active additive penetrates to at least about 10, 20, 25, 30, 35, 40, 45, or 50 microns into the skin with one washing with a leave-on formulation containing the additive. In some embodiments, the active additive penetrates more than about 10, 20, 25, 30, 35, 40, 45, 50, 60, 70, 80, 90, 100, 120, or 150 microns into the skin. In some embodiments this penetration occurs with a single application. In some embodiments this penetration occurs with repeated applications.

Any additive described herein, e.g., sunscreen additives, generally as a component of a bodywash or a leave on formulation, may be used in the methods of the invention. In some embodiments, the additive is a non-sunscreen additive and is encapsulated, e.g., in the form of sol-gel microcapsules. In these embodiments, the additive may be used in combination with a bodywash or a non-bodywash vehicle, such as a skin lotion, gel, cream, and the like, as are well-known in the art.

While it is ordinarily preferred to use the bodywash compositions of the present invention in a manner similar to ordinary soap (i.e., wetting, application of composition, rinsing), it is also anticipated that the composition may be used by application without wetting followed by removal through, for example, wiping. This is the case for soapless cleansers.

One aspect of the invention is a method of applying an active compound to a surface, for example, skin or hair comprising; providing a composition comprising an active compound encapsulated into a sol-gel microcapsule having a high zeta potential; and applying the composition to the surface, for example, skin or hair. Any of the high zeta potential microcapsules described above can be used in the method.

The microcapsules can be formulated to break open in various types of conditions including friction, temperature, light, pH, enzymes, or some combination of these. The capsule can include components that break down when exposed to these conditions, causing release of the contents. In some cases, the components are released immediately upon initial application to a surface including skin or hair. In some embodiments, 1, 2, 5, 10, 20, 30, 40, 50, 60, 70, 80, 90 percent or more of the encapsulated active is released substantially on contact with a surface including skin or hair. In some cases the actives are released over time. In some cases it is desirable to have the active is released quickly, in other cases, it is desired that the active be released over a long period of time. The release can be controlled by controlling the permeability of the capsule to the additive, including controlling the porosity of the capsule. The release can also be controlled by controlling the amount of breakage of the capsules over time. In some embodiments 1, 2, 5, 10, 20, 25, 30, 40, 50, 60, 70, 75, 80, 90, percent or more of the encapsulated active is released within 10 minutes of exposure to the surface. In some embodiments 1, 2, 5, 10, 20, 25, 30, 40, 50, 60, 70, 75, 80, 90 percent or more of the encapsulated active is released within 10 minutes of exposure to the skin. In some embodiments 1, 2, 5, 10, 20, 25, 30, 40, 50, 60, 70, 75, 80, 90 percent or more of the encapsulated active is released within 30 minutes of exposure to the skin. In some embodiments 1, 2, 5, 10, 20, 25, 30, 40, 50, 60, 70, 75, 80, 90 percent or more of the encapsulated active is released within 1 hour of exposure to the skin. In some embodiments 1, 2, 5, 10, 20, 25, 30, 40, 50, 60, 70, 75, 80, 90 percent or more of the encapsulated active is released within 4 hours of exposure to the skin. In some embodiments 1, 2, 5, 10, 20, 25, 30, 40, 50, 60, 70, 75, 80, 90 percent or more of the encapsulated active is released within 6 hours of exposure to the skin. In some embodiments 1, 2, 5, 10, 20, 25, 30, 40, 50, 60, 70, 75, 80, 90 percent or more of the encapsulated active is released within 8 hours of exposure to the skin. In some embodiments 1, 2, 5, 10, 20, 25, 30, 40, 50, 60, 70, 75, 80, 90 percent or more of the encapsulated active is released within 12 hours of exposure to the skin. In some embodiments 1, 2, 5, 10, 20, 25, 30, 40, 50, 60, 70, 75, 80, 90 percent or more of the encapsulated active is released within 24 hours of exposure to the skin. In some embodiments 1, 2, 5, 10, 20, 25, 30, 40, 50, 60, 70, 75, 80, 90 percent or more of the encapsulated active is released within 48 hours of exposure to the skin. In some cases, all of the release is due to breakage of the capsules. These are cases where the capsule shell is substantially impermeable with respect to the active ingredient. In other cases, 1, 2, 5, 10, 20, 25, 30, 40, 50, 60, 70, 75, 80, 90 percent or more of the release is due to the breakage of the capsules.

In some cases, the surface can be pre-treated or post-treated with an agent that will cause the sol-gel capsules to break or will prevent the sol-gel capsules from breaking when they come into contact with the treated surface or are residing on the surface. The pre- or post-treatment agent can either be a gel, crème, lotion or solid or other coating which may contain an substance which can react with or modify the sol-gel capsules either enzymatically, or by pH change, light, pressure or other type of biochemical or physical influence to release or modify the sol-gel capsules. The agent may, for example, release the capsules content or manipulate the way the capsule effects the substrate by either bonding or surrounding the area in which the capsule is present.

The amount of release can be measured either by measuring the surface, such as hair or skin directly, or by obtaining samples by a strip test, or by rubbing the surface, such as skin with a pad containing water or a solvent. The strip test involves adhering an adhesive strip to the surface such as skin or hair and subsequently removing it. The adhesive strip will have a portion of the surface such as hair and skin bound to it and can be either directly analyzed, for instance by a light microscope or electron microscope, or can be extracted, and the presence of the capsules and or the ingredients can be measured. The strip test followed by microscopy also allows the breakage of the capsules to be measured by counting the broken and intact capsules. The rubbing method allows for the quantitation of material on the surface by measuring what has been rubbed off. The amount removed can be controlled by the extent of rubbing and the solvent used. In some cases, water is used, and the rubbing method can be used to determine how strongly the capsules and additives are bound. In other cases, solvents such as methanol, ethanol, or chloroform can be used which can be chosen to extract a substantial amount of the material on the surface such as skin or hair. The extract can be analyzed, for example with chromatographic methods such as HPLC. In some cases, dyes or other indicators, such as fluorescent dyes can be added to the topical formulations in order to assist in the measurement.

A related measurement method is the rinse method. The rinse method involves carrying out successive rinse steps on an area of surface such as skin in order to determine the amount of active and number of capsules that remain bound. A rinse method is described above for the measurement of SPF. This method can also be adapted to be used with other actives and for use with other surfaces.

In some embodiments, pH sensitive polymers can be incorporated to cause release at a given pH. Materials useful for pH-mediated release are known in the art. (see, for example, U.S. Pat. Nos. 7,053,034, 7,098,032, 7,138,382. Polymers that pH-sensitive are have found broad application, for instance, in the area of drug delivery exploiting various physiological and intracellular pH gradients for the purpose of controlled release of drugs. pH sensitivity can be as any change in polymer's physico-chemical properties over certain range of pH. pH-sensitivity usually involves the presence of ionizable groups in the polymer (polyion). Examples of polyions are polyacids, polybases and polyampholytes. Use of pH-sensitive polyacids in drug delivery applications usually relies on their ability to become soluble with the pH increase (acid/salt conversion), to form complex with other polymers over change of pH or undergo significant change in hydrophobicity/hydrophilicity balance. Combinations of all three above factors are also possible.

Copolymers of polymethacrylic acid are polymers which can be insoluble at lower pH but readily solubilized at higher pH. A typical example of pH-dependent complexation is copolymers of polyacrylate(graft)ethyleneglycol which can be formulated into various pH-sensitive hydrogels which exhibit pH-dependent swelling and release. Hydrophobically-modified N-isopropylacrylamide-methacrylic acid copolymer can render regular egg PC liposomes pH-sensitive by pH-dependent interaction of grafted aliphatic chains with lipid bilayer. An example of a polybase for controlled pH release is polyethyleneimine. Polymers with pH-mediated hydrophobicity (like polyethylacrylic acid) can also be used.

C. Business Methods

The invention also encompasses methods of doing business in the field of topical delivery of cosmeceuticals and the transdermal delivery of pharmaceuticals using lathering products, including everyday soap and shampoo, as the delivery agents.

Consumers spend more than $30 billion annually on products that take advantage of topical and transdermal delivery methods. Despite enormous growth in this area, there have been few major innovations. Most delivery methods still rely on lotions, creams or patches. By combining a cosmetic or even pharmaceutical regimen with an activity as routine as washing up or showering, the business methods of the invention capture a significant share of the topical and transdermal delivery market. Products enable personal care product makers to secure a piece of the growing market for cosmeceuticals, like sunscreen, by enhancing existing product lines. They will also enable drug makers to offer consumers more appealing ways to administer prescription and over-the-counter pharmaceuticals

Business methods of the invention encompass a method of doing business comprising marketing an additive for use with an existing bodywash, wherein the additive, when combined with the bodywash, causes an additional effect to the normal effect of the soap or the bodywash. The business methods include methods involving any of the additives described herein, including sunscreens, insect repellants, anti-acne medications, anti-wrinkling agents, deodorants, and all others described herein In some embodiments, the methods include marketing a sunscreen benefit agent (additive) for use with a bodywash, e.g., bar and liquid soaps, and shampoos, to add the benefit of a sunscreen. The sunscreen may be any one of the sunscreen additives described herein. This embodiment is designed to appeal to soap manufacturers looking to broaden the market for their products among the growing population of consumers concerned about skin cancer and wrinkles. Generally, the benefit agent is marketed as a brand-neutral additive for use with existing brands. In some cases, a stand-alone brand may be created.

The sunscreen or other benefit agent may be licensed as an additive, in both liquid and bar soap forms, to personal care product makers of all sizes, to enhance and differentiate their branded product offerings. The license may be exclusive or non-exclusive. If exclusive, it may be exclusive in a defined geographical territory, for a defined time period (often with an option to renew or right of first refusal at the expiration of the time period), for a defined type of skin care product, or any combination of these. The methods also include supplying one or more customers with an option to license or buy the additive, generally for a defined period of time. As with licenses, such an option may be exclusive or non-exclusive. Alternatively, the sunscreen or other benefit agent may be manufactured and supplied to personal care product makers. A further alternative is to manufacture a stand-alone brand of soap/bodywash that includes the additive.

A further component of the business methods of the invention typically includes receiving payment for supplying the additive, license, or the like, to the customer. It will be appreciated that “payment” may be any form of consideration, included monetary consideration. Typically, license payments take the form of an up-front payment, royalties, license maintenance fees or some combination thereof. Also included in payment options are equity in the company receiving the additive or the license to the additive. It will be appreciated that any other form of consideration may also constitute payment in the business methods of the invention

The business methods of the invention may further include manufacturing the additive and/or the additive/bodywash. In some embodiments, different entities perform different aspects; for example, a first entity may manufacture the additive and a second entity may market and/or distribute it. In some embodiments, a single entity performs both manufacturing and marketing.

Business methods of the invention further include a method including the steps of: a) designing an additive for use in a personal care product; b) testing the additive for safety and effectiveness in humans; c) arranging for distribution and marketing of the additive. In some embodiments, steps a) and c) are performed by a first entity, typically a business entity, and step b) is performed by a second entity, such as a business entity or an academic entity. In some or these embodiments, step b) is performed as a joint venture between the two entities.

All publications and patent applications mentioned in this specification are herein incorporated by reference to the same extent as if each individual publication or patent application was specifically and individually indicated to be incorporated by reference.

It will be apparent to one of ordinary skill in the art that many changes and modification can be made to the disclosures presented herein without departing from the spirit or scope of the appended claims.

### EXAMPLES

### Example 1

A sunscreen additive for addition to a bodywash was prepared as follows: To 13.7 g water was added 1 g of polyquaternium-4 (CELQUAT-200), 1.5 gm of MOISTUREGUARD, and 12 g of UV PEARLS. The mixture was stirred until uniform, to produce Phase I. Separately, 1 g of PARSOL 1789 was added to 4 g of Octocrylene with heating, and stirred until uniform, to produce Phase II. Phase I and Phase II were combined with gentle agitation until uniform to produce Phase III, a sunscreen additive.

The sunscreen additive of Phase III was added to 64.5 g of SUAVE Bodywash and stirred until uniform. Finally, 2.3 g of titanium dioxide were added with stirring. The final composition was a sunscreen/bodywash.

### Example 2

The sunscreen/bodywash of Example 1 was tested for SPF capability as follows: 50 cm² of testing site was wetted with 10 ml of water delivered with a syringe. The test sample was applied as per FDA monograph C.F.R. 21 to the area. Lather was worked into the subject for 3 minutes to allow the product to absorb into the skin. The area was rinsed after 2 additional minutes with 20 ml of water, then the area was patted dry and allowed 15 minutes before exposure to radiation as per FDA monograph. The skin was exposed to UV radiation and the MED was noted and compared to the MED for skin without treatment. Results are shown in the Table below.

TABLE
(Lather Method*)

| | | | | | MED | MED | STD | |
|---|---|---|---|---|---|---|---|---|
| Subject | | MED/ | I | Skin | I | II | (8% | SPE |
| ID # | Sex | Hr | (Amps) | Type | J/M² | J/M² | HMS) | Value |
| 46 8676 | F | 127.8 | 7.0 | II | 46.20 | 46.20 | 4.40 | 15.00 |
| 50 3379 | F | 126.4 | 7.0 | II | 46.20 | 46.20 | 4.00 | 18.00 |
| 36 0202 | F | 125.8 | 7.0 | II | 46.20 | 46.20 | 4.40 | 21.60 |
| 56 2392 | F | 125.8 | 7.0 | II | 46.20 | 46.20 | 4.00 | 18.00 |
| 50 1415 | F | 125.8 | 7.0 | II | 46.20 | 46.20 | 4.40 | 21.60 |

| | | |
|---|---|---|
| MEAN (x) | 4.24 | 18.84 |
| STANDARD DEV (s) | 0.22 | 2.80 |
| STD. ERROR | 0.10 | 1.25 |
| S.E. % OF MEAN | 2.36 | 6.63 |
| N | 5 | 5 |

MED: Minimal Erythemal Dose
I: Intensity of light source

This Example demonstrates that the sunscreen/bodywash enhanced the sun protection as measured by this protocol, as compared to untreated skin, by an average SPF of over 18.

### Example 3

A sunscreen/bodywash is prepared by mixing the following ingredients: 0.1 to 7.5 parts by weight of octylmethoxy cinnamate, 0.1 to 6 parts by weight of octyl salicylate, 0.1 to 5 parts by weight of oxybenzone, 1 to 10 parts by weight of cationic surfactant, 0.01 to 1 part by weight of a quaternized compound and 0.01 to 1 part by weight of a preservative.

### Example 4

A sunscreen/bodywash is prepared by mixing the following ingredients:
Water 20-65%
Polyquat 4 0.01-3.75%
Dimethicone 0.01-7%
Octylmethoxycinnamate in amorphous silica
Petrolatum 0.01-10%
Titanium Dioxide 0.01-20%
Octocrylene 0.01-10%
Parsol 1789(Avobenzone) 0.01-3%
Kathon 0.01-2%
Bodywash generic 5-99%

### Example 5

### Highly Charged Microcapsules from Oil-in-Water (O/W) Emulsion

### Encapsulation of Homosal, Vitamin A and Vitamin E

First the non-polar ingredients homomethyl salicylate (Homosal) (18-22 parts) and a mixture of Vitamin A and Vitamin B, (0.5-2 parts) are combined, added with deionized water (50-60 parts) and mixed with a PT 3100 mixer at 6,000 rpm for about 15 minutes at a temperature of about 65° C. to form an emulsion. Aliquots of the emulsion are removed and analyzed by microscope to estimate the droplet size. To the emulsion is added sodium lauryl sulfate (0.08-0.16 parts). Copolymer surfactant Atlox 49-12 (0.05 to 0.10) parts can also be added. Tetraethyl ortho silicate (TEOS) (15-25 parts) is added to the emulsion. Polyquaternium-4 (0.03 to 0.5 parts) is added to the emulsion. A 10% HCl solution is then added a drop at a time while mixing the emulsion to bring the pH to about 3.8. The emulsion is then mixed for about 2 to 2.5 hours while the TEOS hydrolyzes and the sol-gel capsules are formed. The pH is monitored, and adjusted to pH 3.8 if needed. An aliquot of capsules in solution can be removed and the zeta potential of the capsules determined. If the zeta potential is lower than desired, the capsules can be treated with a cationic agent such as polyquatemium-4 in order to increase the zeta potential on the particles. The reaction mixture is filtered with a Buchner funnel using a 1 micron filter. The capsules are rinsed 2-3 times with deionized water. The moist capsules are then placed in an oven at 40° C.-55° C. for 24 to 48 hours to dry the capsules.

In an alternative embodiment of the above example, the Polyquaternium-4 (0.03 to 0.5 parts) is added to the reaction mixture after the capsules have been formed rather than being added to the emulsion before formation of the capsules.

In another alternative embodiment of the above example, the Polyquaternium-4 (0.03 to 0.5 parts) is dissolved in an aqueous solution, which is applied to the capsules after they are dried. After the addition of the Polyquaternium-4, the moist capsules are placed in an oven at 40° C.-55° C. for 24 to 48 hours a second time to dry the charged capsules.

### Example 6

### Highly Charged Microcapsules from a Water-in-Oil (W/O) Emulsion Encapsulation of Glycerin

Glycerin (10-20 parts), water (10-20 parts), and siloxane fluid (Dow Corning 245) (45-55 parts), and sorbitan oleate surfactant (Crill 3 NF) (0.08-0.16) are combined and mixed with a PT 3100 mixer at 2,000-4,000 rpm for about 10 minutes at a temperature of about 55° C. to form a water-in-oil emulsion. Aliquots of the emulsion are removed and analyzed by microscope to estimate the droplet size. A copolymer surfactant with HLB value of 2 to 6 can also be added if needed to stabilize the emulsion. Tetraethyl ortho silicate (TEOS) (15-25 parts) is added to the emulsion. Polyquaternium-4 (0.03 to 0.5 parts) is added to the emulsion. A 10% HCl solution is then added a drop at a time while mixing the emulsion to bring the pH to about 3.8. The emulsion is then mixed for about 1 to 2 hours while the TEOS hydrolyzes and the sol-gel capsules are formed. The pH is monitored, and adjusted to pH 3.8 if needed. The reaction mixture is filtered with a Buchner funnel using a 1 micron filter. An aliquot of capsules in solution can be removed and the zeta potential of the capsules determined. If the zeta potential is lower than desired, the capsules can be treated with a cationic agent such as polyquaternium-4 in order to increase the zeta potential on the particles. The capsules are rinsed 2-3 times with deionized water. The moist capsules are then placed in an oven at 40° C.-55° C. for 24 to 48 hours to dry the capsules.

In an alternative embodiment of the above example, the Polyquaternium-4 (0.03 to 0.5 parts) is added to the reaction mixture after the capsules have been formed rather than being added to the emulsion before formation of the capsules.

In another alternative embodiment of the above example, the Polyquaternium-4 (0.03 to 0.5 parts) is dissolved in an aqueous solution, which is applied to the capsules after they are dried. After the addition of the Polyquaternium-4, the moist capsules are placed in an oven at 40° C.-55° C. for 24 to 48 hours a second time to dry the charged capsules.

### Example 7

### Highly Charged Microcapsules from an Aqueous Solution with Phospholipid Template Encapsulation of Glycerin

Deionized water (45-55 parts), Glycerin (5-15 parts), and phospholipid (Phospholipon 85G) (18-28 parts) are combined and mixed with a PT 3100 mixer at 3,000-6,000 rpm for about 10 minutes at a temperature of about 42° C.-65° C. to form an aqueous solution comprising liposomes. Tetraethyl ortho silicate (TEOS) (15-25 parts) is added to the reaction mixture. Polyquaternium-4 (0.03 to 0.5 parts) is added to the reaction mixture. A 10% H2SO₄ solution is then added a drop at a time while mixing the emulsion to bring the pH to about 3.4. The emulsion is then mixed for about 1 to 2 hours while the TEOS hydrolyzes and the sol-gel capsules are formed. The pH is monitored, and adjusted to pH 3.4 if needed. An aliquot of capsules in solution can be removed and the zeta potential of the capsules determined. If the zeta potential is lower than desired, the capsules can be treated with a cationic agent such as polyquaternium-4 in order to increase the zeta potential on the particles. The reaction mixture is filtered with a Buchner funnel using a 1 micron filter. The capsules are rinsed 2-3 times with deionized water. The moist capsules are then placed in an oven at 40° C.-55° C. for 24 to 48 hours to dry the capsules.

In an alternative embodiment of the above example, the Polyquaternium-4 (0.03 to 0.5 parts) is added to the reaction mixture after the capsules have been formed rather than being added to the emulsion before formation of the capsules.

In another alternative embodiment of the above example, the Polyquatemium-4 (0.03 to 0.5 parts) is dissolved in an aqueous solution, which is applied to the capsules after they are dried. After the addition of the Polyquaternium-4, the moist capsules are placed in an oven at 40° C.-55° C. for 24 to 48 hours a second time to dry the charged capsules.

In the above method, phospholipid liposomes are used as the templates. In a variation of the above method, polymer microcapsules such those formed from polystyrene, hydroxyethylcellulose, polyacrylamide, where the polymer microcapsule comprises an active ingredient, can be used as templates to form highly charged microcapsules.

While certain embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A sol-gel microcapsule with a zeta potential of at least about 40 mV.

2. The sol-gel microcapsule of claim 1, wherein the zeta potential is at least about 50 mV.

3-4. (canceled)

5. A plurality of sol-gel microcapsules capable of binding to a surface wherein an average of at least about 50% of the microcapsules remain bound to the surface for an average of greater than at least about 4 hours.

6. The sol-gel microcapsule of claim 1 wherein the microcapsule comprises a cationic agent.

7. The sol-gel microcapsule of claim 6 wherein the cationic agent comprises a cationic polymer.

8. The sol-gel microcapsule of claim 7 wherein the cationic polymer comprises polyquaternium-4, -7, -11, -22, -27, -44, 51, or -64.

9. The sol-gel microcapsule of claim 7 wherein the cationic polymer comprises polyquaternium-4.

10. The sol-gel microcapsule of claim 1 wherein the microcapsule is associated with an additive.

11. The sol-gel microcapsule of claim 10 wherein the additive is encapsulated in the microcapsule.

12. The sol-gel microcapsule of claim 10 wherein the additive is located substantially within the sol-gel microcapsule.

13. The sol-gel microcapsule of claim 10, wherein the additive is selected from the group consisting of steroidal anti-inflammatory actives, analgesic actives, antifungals, antibacterials, antiparasitics, anti-virals, anti-allergenics, anti-cellulite additives, medicinal actives, skin rash, skin disease and dermatitis medications, insect repellant actives, antioxidants, hair growth promoter, hair growth inhibitor, hair bleaching agents, deodorant compounds, sunless tanning actives, skin lightening actives, anti-acne actives, anti-skin wrinkling actives, anti-skin aging actives, vitamins, nonsteroidal anti-inflammatory actives, anesthetic actives, anti-pruritic actives, anti-microbial actives, dental care agents, personal care agents, nutraceuticals, pharmaceuticals, fragrances, antifouling agents, pesticides, lubricants, etchants, and mixtures and combinations thereof.

14. The sol-gel microcapsule of claim 10 wherein the additive is selected from the group consisting of sunscreens, skin lightening actives, anti-aging additives, fragrances, pharmaceuticals, antibacterials, moisturizers, anti-acne actives, and insect repellants.

15. The sol-gel microcapsule of claim 10, wherein the additive comprises a sunscreen.

16. The sol-gel microcapsule of claim 15, wherein the sunscreen is selected from the group consisting of aminobenzoic acid, avobenzone, cinnoxate, dioxybenzone, homosalate, menthyl anthranilate, octocrylene, octyl methoxycinnamate, octyl salicylate, oxybenzone, padimate O, phenylbenzimidazole sulfonic acid, sulisobenzone, and trolamine salicylate.

17. The sol-gel microcapsule of claim 15, wherein the sunscreen comprises a UVA-absorbing sunscreen, a UVB-absorbing sunscreen, and a physical blocker sunscreen.

18. The sol-gel microcapsule of claim 17 wherein (i) the UVB-absorber sunscreen is selected from the group consisting of aminobenzoic acid, cinoxate, dioxybenzone, homosalate, octocrylene, octyl methoxycinnamate, octyl salicylate, oxybenzone, padimate O, phenylbenzimidazole sulfonic acid, sulisobenzone, and trolamine salicylate; (ii) the UVA-absorber sunscreen is selected from the group consisting of avobenzone and menthyl anthranilate; and (iii) the physical blocker sunscreen is selected from the group consisting of titanium dioxide and zinc oxide.

19. A composition comprising the sol-gel microcapsule of claim 10, and further comprising a vehicle suitable for treatment of surfaces in topical, agricultural, textile, industrial, transportation, marine, pharmaceutical, or personal care.

20. The composition of claim 19 wherein the composition comprises a wash-on product.

21. The composition of claim 19 wherein the composition comprises a leave-on product.

22. The composition of claim 19 wherein the microcapsules in the composition experience an average of greater than about 50% breakage when applied to the surface.

23. The composition of claim 22 wherein the breakage substantially occurs on initial application to the surface.

24. The composition of claim 22 wherein the average of greater than 50% breakage occurs over a period of about 1 hour.

25-27. (canceled)

28. The composition of claim 22 wherein the breakage occurs due to the conditions of surface application.

29. The composition of claim 28 wherein the condition of surface application is friction, pressure, light, pH change, or enzymatic action.

30. A method of applying an active compound to a surface comprising;
providing a composition comprising an active compound encapsulated into a sol-gel microcapsule having a zeta potential of greater than about 30 mV; and applying the composition to the surface.

31. The method of claim 30 wherein the zeta potential is greater than 30 mV.

32-33. (canceled)

34. The method of claim 30 wherein the zeta potential is greater than 60 mV.

35. The method of claim 30 wherein the capsules comprise a cationic polymer.

36. The method of claim 35 wherein the cationic polymer comprises a polyquaternium.

37. The method of claim 35 wherein the cationic polymer comprises polyquaternium-4, -7, -11, -22, -27, -44, 51, or -64.

38. The method of claim 30, wherein the additive is selected from the group consisting of steroidal anti-inflammatory actives, analgesic actives, antifungals, antibacterials, antiparasitics, anti-virals, anti-allergenics, anti-cellulite additives, medicinal actives, skin rash, skin disease and dermatitis medications, insect repellant actives, antioxidants, hair growth promoter, hair growth inhibitor, hair bleaching agents, deodorant compounds, sunless tanning actives, skin lightening actives, anti-acne actives, anti-skin wrinkling actives, anti-skin aging actives, vitamins, nonsteroidal anti-inflammatory actives, anesthetic actives, anti-pruritic actives, anti-microbial actives, dental care agents, personal care agents, nutraceuticals, pharmaceuticals, fragrances, antifouling agents, pesticides, lubricants, etchants, and mixtures and combinations thereof.

39. The method of claim 30 wherein the additive is selected from the group consisting of sunscreens, skin lightening actives, anti-aging additives, fragrances, pharmaceuticals, antibacterials, moisturizers, anti-acne actives, and insect repellants.

40. The method of claim 30 wherein the additive comprises a sunscreen.

41. The method of claim 30 wherein the sunscreen is selected from the group consisting of aminobenzoic acid, avobenzone, cinnoxate, dioxybenzone, homosalate, menthyl anthranilate, octocrylene, octyl methoxycinnamate, octyl salicylate, oxybenzone, padimate O, phenylbenzimidazole sulfonic acid, sulisobenzone, and trolamine salicylate.

42. The method of claim 30 wherein the sunscreen comprises a UVA-absorbing sunscreen, a UVB-absorbing sunscreen, and a physical blocker sunscreen.

43. The method of claim 30 wherein (i) the UVB-absorber sunscreen is selected from the group consisting of aminobenzoic acid, cinoxate, dioxybenzone, homosalate, octocrylene, octyl methoxycinnamate, octyl salicylate, oxybenzone, padimate O, phenylbenzimidazole sulfonic acid, sulisobenzone, and trolamine salicylate; (ii) the UVA-absorber sunscreen is selected from the group consisting of avobenzone and menthyl anthranilate; and (iii) the physical blocker sunscreen is selected from the group consisting of titanium dioxide and zinc oxide.

44. The method of claim 30 wherein the microcapsules in the composition experience an average of greater than about 50% breakage when applied to the surface.

45. The method of claim 30 wherein the breakage substantially occurs on initial application to the surface.

46. The method of claim 30 wherein the breakage occurs over a period of 1 hour.

47-49. (canceled)

50. A method of manufacturing a highly charged sol-gel microcapsule comprising a non-polar active ingredient comprising:
(a) combining the non-polar active ingredient, optional non-polar diluent, and aqueous phase;
(b) agitating the combination formed in (a) to form an oil-in-water (O/W) emulsion wherein the non-polar active ingredient and optional non-polar diluent comprise the dispersed phase;
(c) adding one or more surfactants;
(d) adding a cationic agent;
(e) adding a gel precursor to the O/W emulsion; and
(f) mixing the composition from step (e) while the gel precursor hydrolyzes and sol-gel capsules are formed which comprise the non-polar active ingredient.

51. The method of claim 50 further comprising step (g) filtering the sol-gel microcapsules and step (h) rinsing the sol-gel microcapsules.

52. The method of claim 51 further comprising step (i) drying the microcapsules.

53. The method of claim 50 wherein the method of manufacturing produces a microcapsule having zeta potential of at least about 30 mV.

54-55. (canceled)

56. The method of claim 50 wherein the method of manufacturing produces a microcapsule having zeta potential of at least about 60 mV.

57. The method of claim 50 wherein the steps are carried out in the order listed.

58. The method of claim 50 wherein the cationic agent is added after the addition of the gel precursor.

59. The method of claim 50 wherein the cationic agent is added during step (f).

60. The method of claim 50 wherein the cationic agent is added after step (f).

61. The method of claim 51 wherein the cationic agent is added during step (h) of rinsing the sol-gel microcapsules.

62. The method of claim 52 wherein the cationic agent is added after step (i) of drying the sol-gel microcapsules.

63. The method of claim 50 wherein the cationic agent comprises a cationic polymer.

64. The method of claim 63 wherein the cationic polymer comprises polyquaternium-4, -7, -11, -22, -27, -44, 51, or -64.

65. The method of claim 64, wherein the cationic polymer comprises polyquaternium-4.

66. The method of claim 50 wherein the cationic agent comprises a proton donor.

67. The method of claim 50 wherein step (f) is carried out at acidic pH.

68. The method of claim 67 wherein step (f) is carried out at a pH from 3.6 to 4.0.

69. The method of claim 50 wherein the one or more surfactants comprises a copolymer surfactant.

70. The method of claim 50 wherein the one or more surfactants have a combined hydrophile-lipopbile balance (HLB) of between 9 and 11.

71. A method of manufacturing a highly charged sol gel microcapsule comprising a polar active ingredient comprising:
(a) combining the polar active ingredient, water, optional polar diluent, and a non-polar (oil) phase;
(b) agitating the combination formed in (a) to form an water-in-oil (W/O) emulsion wherein the polar active ingredient, water, and optional polar diluent comprise the dispersed phase;
(c) adding one or more surfactants;
(d) adding a cationic agent;
(e) adding a gel precursor to the W/O emulsion; and
(f) mixing the composition from step (e) while the gel precursor hydrolyzes and sol-gel capsules are formed which comprise the polar active ingredient.

72. The method of claim 71 further comprising step (g) filtering the sol-gel microcapsules and step (h) rinsing the sol-gel microcapsules.

73. The method of claim 52 further comprising step (i) drying the microcapsules.

74. The method of claim 71 wherein the method of manufacturing produces a microcapsule having zeta potential of at least 30 mV.

75-76. (canceled)

77. The method of claim 71 wherein the method of manufacturing produces a microcapsule having zeta potential of at least 60 mV.

78. The method of claim 71 wherein the steps are carried out in the order listed.

79. The method of claim 71 wherein the cationic agent is added after the addition of the gel precursor.

80. The method of claim 71 wherein the cationic agent is added during step (f).

81. The method of claim 71 wherein the cationic agent is added after step (f).

82. The method of claim 72 wherein the cationic agent is added during step (h) of rinsing the sol-gel microcapsules.

83. The method of claim 73 wherein the cationic agent is added after step (i) of drying the sol-gel microcapsules.

84. The method of claim 71 wherein the cationic agent comprises a cationic polymer.

85. The method of claim 84 wherein the cationic polymer comprises polyquaternium-4, -7, -11, -22, -27, -44, 51, or -64.

86. The method of claim 85, wherein the cationic polymer comprises polyquaternium-4.

87. The method of claim 71 wherein the cationic agent comprises a proton donor.

88. The method of claim 71 wherein step (f) is carried out at acidic pH.

89. The method of claim 88 wherein step (f) is carried out at a pH from 3.6 to 4.0.

90. The method of claim 71 wherein the one or more surfactants comprises a copolymer surfactant.

91. The method of claim 71 wherein the one or more surfactants have a combined hydrophile-lipophile balance (HLB) of between 2 and 6.

92. A method of forming a highly charged sol-gel microcapsule comprising an active ingredient within a template comprising:
(a) forming a dispersion of templates, wherein the templates comprise an active ingredient, in an aqueous continuous phase;
(b) adding a cationic agent;
(c) adding a gel precursor to the aqueous continuous phase; and
(d) mixing the composition from step (c) while the gel precursor hydrolyzes and sol-gel capsules are formed.

93. The method of claim 92 further comprising step (e) filtering the sol-gel microcapsules and step (f) rinsing the sol-gel microcapsules.

94. The method of claim 93 further comprising step (g) drying the microcapsules.

95. The method of claim 92 wherein the method of manufacturing produces a microcapsule having zeta potential of at least 30 mV.

96-97. (canceled)

98. The method of claim 92 wherein the method of manufacturing produces a microcapsule having zeta potential of at least 60 mV.

99. The method of claim 92 wherein the steps are carried out in the order listed.

100. The method of claim 92 wherein the cationic agent is added after the addition of the gel precursor.

101. The method of claim 92 wherein the cationic agent is added during step (c).

102. The method of claim 92 wherein the cationic agent is added after step (c).

103. The method of claim 93 wherein the cationic agent is added during step (f) of rinsing the sol-gel microcapsules.

104. The method of claim 94 wherein the cationic agent is added after step (g) of drying the sol-gel microcapsules.

105. The method of claim 92 wherein the cationic agent comprises a cationic polymer.

106. The method of claim 105 wherein the cationic polymer comprises polyquatemium-4, -7, -11, -22, -27, -44, 51, or -64.

107. The method of claim 106, wherein the cationic polymer comprises polyquaternium-4.

108. The method of claim 92 wherein the cationic agent comprises a proton donor.

109. The method of claim 92 wherein step (d) is carried out at acidic pH.

110. The method of claim 92 wherein step (d) is carried out at a pH from 3.6 to 4.0.

111. The method of claim 92 wherein the template comprises a microsphere.

112. The method of claim 92 wherein the template comprises a polymer, liposome or micelle.

113. The method of claim 112 wherein the template comprises a phospholipid.
